(19) United States
(12) Patent Application Publication
(10) Pub. No.: US 2023/0348906 A1
(43) Pub. Date: Nov. 2, 2023

(54) DUX4 INHIBITORS AND METHODS OF USE THEREOF

(71) Applicants: ULTRAGENYX PHARMACEUTICAL INC., Novato, CA (US); SAINT LOUIS UNIVERSITY, St. Louis, MO (US)

(72) Inventors: Sean Christopher DAUGHERTY, Petaluma, CA (US); Lishan CHEN, San Diego, CA (US); Francis Michael SVERDRUP, Lake St. Louis, MO (US)

(21) Appl. No.: 18/043,520

(22) PCT Filed: Sep. 1, 2021
(86) PCT No.: PCT/US21/48611

§ 371 Date: Feb. 28, 2023

Related U.S. Application Data
(60) Provisional application No. 63/073,304, filed on Sep. 1, 2020.

(51) Int. Cl. C12N 15/113; A61K 47/54; A61K 47/68; A61P 21/00

(52) U.S. Cl. CPC C12N 15/113; A61K 47/543; A61K 47/6849; A61P 21/00

(57) ABSTRACT

This application relates to double-stranded small interfering RNAs that modulate DUX4 gene expression and describes methods of inhibiting DUX4 gene expression by contacting a cell with said double-stranded small interfering RNAs. The application further provides compositions comprising said double-stranded small interfering RNAs and their use in methods of preventing or treating a disease or disorder associated with aberrant expression of DUX4, such as facioscapulohumeral dystrophy (FSHD) or cancer, in a subject.

### CROSS-REFERENCE TO RELATED APPLICATIONS

This application claims the benefit of U.S. Provisional Application Ser. No. 63/073,304, filed Sep. 1, 2020, the contents of which are hereby incorporated by reference in their entirety.

### REFERENCE TO A SEQUENCE LISTING

This application contains a Sequence Listing submitted electronically as a text file by EFS-Web. The text file, named “8957-5-PCT_Seq_Listing_ST25.txt”, has a size in bytes of 194,000 bytes, and was recorded on 25 Aug. 2021. The information contained in the text file is incorporated herein by reference in its entirety pursuant to 37 CFR § 1.52(e)(5).

### TECHNICAL FIELD OF THE INVENTION

This invention relates to double-stranded small interfering RNAs (siRNAs) that modulate DUX4 gene expression, and their applications in research, diagnostics, and/or therapeutics. In some embodiments, it relates to compositions and methods comprising the said siRNAs in the prevention and/or treatment of facioscapulohumeral muscular dystrophy (FSHD).

### BACKGROUND OF THE INVENTION

Facioscapulohumeral muscular dystrophy (FSHD) is a rare genetic disease affecting about one in 10,000 people worldwide. FSHD patients exhibit progressive, asymmetric muscle weakness and up to 20% of affected individuals become severely disabled. Non-muscular symptoms include subclinical sensorineural hearing loss telangiectasia.

Aberrant expression of the DUX4 protein in skeletal muscle due to inefficient epigenetic repression of the DUX4 gene is thought to cause FSHD. DUX4 is a retrogene encoded in each unit of the D4Z4 macrosatellite repeat array. D4Z4 repeats are bi-directionally transcribed in somatic tissues and generate long stretches of RNA and small RNA fragments that may have a role in epigenetic silencing. The more prevalent form of FSHD (FSHD1) is caused by the deletion of a subset of D4Z4 macrosatellite repeats in the subtelomeric region of chromosome 4q. Unaffected individuals have 11-100 of the 3.3 kb D4Z4 repeat units, whereas FSHD1 individuals have 10 or fewer repeats. FSHD2 is associated with decreased DNA methylation of the D4Z4 repeats on the same 4 qA haplotype. Thus, administration of agents that suppress expression of the DUX4 gene is a promising therapeutic approach for preventing or treating FSHD1 and FSHD2. Beyond their potential utility in the prevention or treatment of FSHD, DUX4-targeted treatments may also improve the success of cancer immunotherapies, as DUX4 expression been found to suppress MHC class I to promote cancer immune evasion and mediate resistance to anti-CTLA-4 therapy. See Chew et al., 2019, Dev Cell 50(5): 525-6.

Double-stranded oligonucleotides have been used to modulate gene expression for use in research, diagnostics, and/or therapeutics. One method of modulation of gene expression is RNA interference (RNAi), which generally refers to gene silencing involving the introduction of double-stranded RNA (dsRNA) leading to the sequence-specific reduction of targeted endogenous mRNA levels. The reduction of target mRNA may occur by one of several different mechanisms, depending on the sequence or structure of the dsRNA. For example, it may lead to degradation of the target mRNA through formation of RNA induced silencing complex (RISC), or transcriptional silencing in which transcription of the mRNA is inhibited in a process called RNA-induced transcriptional silencing (RITS), or by modulation of microRNA (miRNA) function. MicroRNAs are small non-coding RNAs that regulate the expression of messenger RNAs. The binding of an RNAi compound to a microRNA prevents that microRNA from binding to its messenger RNA targets, and thus interferes with the function of the microRNA. The sequence-specificity of RNAi compounds makes them promising candidates as therapeutics to selectively modulate the expression of genes involved in the pathogenesis of diseases.

There continues to be a need in the art for methods and agents for treatment of FSHD. The present application addresses this need by providing oligonucleotides, and compositions and methods comprising them, that can suppress aberrant expression of DUX4 gene and thus can ameliorate, prevent or treat FSHD.

### SUMMARY OF THE INVENTION

The present invention provides double-stranded small interfering RNA (siRNA) molecules for reducing or inhibiting the expression of the DUX4 gene. The present invention also provides a method of reducing or inhibiting expression of DUX4 in a cell comprising contacting the cell with a double-stranded siRNA molecule targeted to DUX4, thereby reducing or inhibiting expression of DUX4. In another aspect, the invention provides compositions and methods for the prevention or treatment of various disorders, including facioscapulohumeral muscular dystrophy (FSHD) by administering the double-stranded siRNA molecules and compositions comprising the same to a subject.

Accordingly, in one aspect, the present invention includes double-stranded small interfering RNA (siRNA) molecules, each molecule comprising a sense strand and an antisense strand, that are useful for reducing or inhibiting the aberrant expression of the DUX4 gene in a cell. In some embodiments, the double-stranded small interfering RNA comprises at least one modified nucleoside.

In some embodiments, the DUX4 gene comprises a nucleobase sequence that is at least 85%, at least 90% identical, or at least 95% identical to SEQ ID NO: 593. In certain embodiments, DUX4 comprises a nucleobase sequence that is 100% identical to SEQ ID NO: 593.

In some embodiments, the antisense strand of the double-stranded small interfering RNA comprises a nucleobase sequence that is at least 85%, at least 90% or at least 95% complementary to an equal length portion of SEQ ID NO: 593. In some embodiments, the antisense strand of the double-stranded small interfering RNA comprises a nucleobase sequence that is 100% complementary to an equal length portion of SEQ ID NO: 593.

In some embodiments, the antisense strand of the double-stranded small interfering RNA comprises a nucleobase sequence that is complementary to at least 8 contiguous nucleobases of an equal length portion of SEQ ID NO: 593. In various embodiments, the antisense strand of the double-stranded small interfering RNA comprises a nucleobase sequence that is complementary to at least 8, at least 9, at least 10, at least 11, at least 12, at least 13, at least 14, at least 15, at least 16, at least 17, at least 18, or at least 19 contiguous nucleobases of an equal length portion of SEQ ID NO: 593.

In one embodiment, the antisense strand of the double-stranded small interfering RNA comprises a nucleobase sequence that is complementary to at least 8 contiguous nucleobases of an equal length portion within nucleobases 4605 to 7485 of SEQ ID NO: 593. For example, in various embodiments, the antisense strand of the double-stranded small interfering RNA may comprise a nucleobase sequence that is complementary to at least 8 contiguous nucleobases of an equal length portion within nucleobases 4605-4638, 4693-4727, 4765-4783, 4933-4951, 4990-5011, 5075-5093, 5127-5148, 5161-5193, 5201-5228, 5243-5279, 5305-5327, 5353-5397, 5433-5461, 5464-5509, 5522-5540, 5651-5670, 5809-5830, 5842-5865, 5969-5990, 6066-6086, 6109-6135, 6183-6229, 6328-6369, 6403-6451, 7120-7138, 7162-7190, 7239-7285, 7404-7437, or 7452-7485 of SEQ ID NO: 593. In various embodiments, the antisense strand of the double-stranded small interfering RNA comprises a nucleobase sequence that is complementary to at least at least 8, at least 9, at least 10, at least 11, at least 12, 13, at least 14, at least 15, at least 16, at least 17, at least 18, or at least 19 contiguous nucleobases of an equal length portion of nucleobases 4605 to 7485 of SEQ ID NO: 593, for example, within nucleobases 4605-4638, 4693-4727, 4765-4783, 4933-4951, 4990-5011, 5075-5093, 5127-5148, 5161-5193, 5201-5228, 5243-5279, 5305-5327, 5353-5397, 5433-5461, 5464-5509, 5522-5540, 5651-5670, 5809-5830, 5842-5865, 5969-5990, 6066-6086, 6109-6135, 6183-6229, 6328-6369, 6403-6451, 7120-7138, 7162-7190, 7239-7285, 7404-7437, or 7452-7485 of SEQ ID NO: 593.

In some embodiments, the antisense strand of the double-stranded small interfering RNA comprises a nucleobase sequence comprising at least 8, at least 9, at least 10, at least 11, or at least 12 contiguous nucleobases of any of the nucleobase sequences listed in Table 1, i.e., a sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, and 592. In some embodiments, the antisense strand of the double-stranded small interfering RNA comprises a nucleobase sequence comprising at least 8, at least 9, at least 10, at least 11, or at least 12 contiguous nucleobases of any of the nucleobase sequences selected from the group consisting of SEQ ID NOs: 216, 218, 220, 312, 324, 340, 342, 348, 350, 352, 354, 364, 372, 376, 400, 402, 404, 410, 434, 446, 448, 450, 462 and 564. In some embodiments, the antisense strand of the double-stranded small interfering RNA comprises or consists of a nucleic acid sequence of any one of SEQ ID NOs: 216, 218, 220, 312, 324, 340, 342, 348, 350, 352, 354, 364, 372, 376, 400, 402, 404, 410, 434, 446, 448, 450, 462 and 564.

In some embodiments, the antisense strand of the double-stranded small interfering RNA comprises a nucleobase sequence comprising at least 8, at least 9, at least 10, at least 11, or at least 12 contiguous nucleobases of any of the nucleobase sequences listed in Table 5, i.e., a sequence selected from the group consisting of SEQ ID NOs: 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, and 688. In some embodiments, the antisense strand of the double-stranded small interfering RNA comprises a nucleobase sequence comprising at least 8, at least 9, at least 10, at least 11, or at least 12 contiguous nucleobases of any of the nucleobase sequences selected from the group consisting of SEQ ID NOs: 602, 604, 606, 616, 684, 686, and 688. In some embodiments, the antisense strand of the double-stranded small interfering RNA comprises or consists of a nucleic acid sequence of any one of SEQ ID NOs: 602, 604, 606, 616, 684, 686, and 688.

In some embodiments, the sense strand of the double-stranded small interfering RNA comprises a nucleobase sequence at least 85% complementary to the antisense strand of the double-stranded small interfering RNA. In various embodiments, the sense strand of the double-stranded small interfering RNA comprises a nucleobase sequence at least 90%, at least 95%, or 100% complementary to the antisense strand of the double-stranded small interfering RNA.

In some embodiments, the sense strand of the double-stranded small interfering RNA comprises a nucleobase sequence that is identical to at least 8 contiguous nucleobases of an equal length portion within nucleobases 4605 to 7485 of SEQ ID NO: 593. For example, in various embodiments, the sense strand of the double-stranded small interfering RNA comprises a nucleobase sequence that is identical to at least 8 contiguous nucleobases of an equal length portion within nucleobases 4605-4638, 4693-4727, 4765-4783, 4933-4951, 4990-5011, 5075-5093, 5127-5148, 5161-5193, 5201-5228, 5243-5279, 5305-5327, 5353-5397, 5433-5461, 5464-5509, 5522-5540, 5651-5670, 5809-5830, 5842-5865, 5969-5990, 6066-6086, 6109-6135, 6183-6229, 6328-6369, 6403-6451, 7120-7138, 7162-7190, 7239-7285, 7404-7437, or 7452-7485 of SEQ ID NO: 593. In various embodiments, the sense strand of the double-stranded small interfering RNA comprises a nucleobase sequence that is identical to at least 8, at least 9, at least 10, at least 11, at least 12, at least 13, at least 14, at least 15, at least 16, at least 17, at least 18, or at least 19 contiguous nucleobases of an equal length portion of nucleobases 4605 to 7485 of SEQ ID NO: 593, for example, nucleobases 4605-4638, 4693-4727, 4765-4783, 4933-4951, 4990-5011, 5075-5093, 5127-5148, 5161-5193, 5201-5228, 5243-5279, 5305-5327, 5353-5397, 5433-5461, 5464-5509, 5522-5540, 5651-5670, 5809-5830, 5842-5865, 5969-5990, 6066-6086, 6109-6135, 6183-6229, 6328-6369, 6403-6451, 7120-7138, 7162-7190, 7239-7285, 7404-7437, or 7452-7485 of SEQ ID NO: 593.

In some embodiments, the sense strand of the double-stranded small interfering RNA comprises a nucleobase sequence comprising at least 8, at least 9, at least 10, at least 11, or at least 12 contiguous nucleobases of any one of the nucleobase sequences listed in Table 1, i.e., a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, and 591. In some embodiments, the sense strand of the double-stranded small interfering RNA comprises a nucleobase sequence comprising at least 8, at least 9, at least 10, at least 11, or at least 12 contiguous nucleobases of any of the nucleobase sequences selected from the group consisting SEQ ID NOs: 215, 217, 219, 311, 323, 339, 341, 347, 349, 351, 353, 363, 371, 375, 399, 401, 403, 409, 433, 445, 447, 449, 461 and 563. In some embodiments, the sense strand of the double-stranded small interfering RNA comprises or consists of a nucleobase sequence of any one of the nucleobase sequences of SEQ ID NOs: 215, 217, 219, 311, 323, 339, 341, 347, 349, 351, 353, 363, 371, 375, 399, 401, 403, 409, 433, 445, 447, 449, 461 and 563.

In some embodiments, the sense strand of the double-stranded small interfering RNA comprises a nucleobase sequence comprising at least 8, at least 9, at least 10, at least 11, or at least 12 contiguous nucleobases of any one of the nucleobase sequences listed in Table 5, i.e., a sequence selected from the group consisting of SEQ ID NOs: 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, and 687. In some embodiments, the antisense strand of the double-stranded small interfering RNA comprises a nucleobase sequence comprising at least 8, at least 9, at least 10, at least 11, or at least 12 contiguous nucleobases of any of the nucleobase sequences selected from the group consisting of SEQ ID NOs: 601, 603, 605, 615, 683, 685, and 687. In some embodiments, the antisense strand of the double-stranded small interfering RNA comprises or consists of a nucleic acid sequence of any one of SEQ ID NOs: 601, 603, 605, 615, 683, 685, and 687.

In some embodiments, the double-stranded small interfering RNA comprises at least one modified nucleoside. In some embodiments, the sense strand of the double-stranded small interfering RNA comprises at least one modified nucleoside. In some embodiments, each nucleoside of the sense strand of the double-stranded small interfering RNA comprises a modified nucleoside.

In some embodiments, at least one nucleoside of the sense strand of the double-stranded siRNA comprises a modified sugar. In some embodiments, each nucleoside of the sense strand of the double-stranded siRNA comprises a modified sugar. In some embodiments, the modified nucleoside comprises a 2′-F modified sugar and/or a 2′-OMe modified sugar. In some embodiments, the modified nucleoside comprises a 2′-OMe modified sugar. In some embodiments, the modified nucleoside comprises a 2′-F modified sugar modified sugar.

In some embodiments, the antisense and/or the sense strand of the double-stranded small interfering RNA comprises a TT overhang at the 3′ end.

In some embodiments, the sense strand of the double-stranded small interfering RNA comprises at least one modified internucleoside linkage. In some embodiments, the sense strand of the double-stranded small interfering RNA comprises at least 2, 3, 4, or 5 modified internucleoside linkages. In some embodiments, the modified internucleoside linkage is a phosphorothioate linkage.

In some embodiments, the double-stranded small interfering RNA is conjugated to a lipophilic molecule, an antibody, an aptamer, a ligand, a peptide, or a polymer. In some embodiments, the lipophilic molecule may be a long chain fatty acid (LCFA). In some embodiments, the antibody is an anti-transferrin receptor antibody.

In another aspect, the present invention includes a pharmaceutical composition comprising a double-stranded small interfering RNA described herein or a salt thereof, and at least one pharmaceutically acceptable carrier. The pharmaceutical composition may be for use in medical therapy. The pharmaceutical composition may be for use in in the treatment of a human or animal body. In another aspect, the present invention includes a use of the pharmaceutical composition for preparing or manufacturing a medicament for ameliorating, preventing, delaying onset, or treating a disease or disorder associated with aberrant expression of DUX4 in a subject need thereof. In another aspect, the present invention includes a method for ameliorating, preventing, delaying onset, or treating a disease or disorder associated with aberrant expression of DUX4 in a subject need thereof by administering the pharmaceutical composition to the subject. The disease or disorder may be facioscapulohumeral muscular dystrophy (FSHD), and may be FSHD1 or FSHD2. In another aspect, the present invention includes a method of ameliorating, preventing, delaying onset, or treating facioscapulohumeral muscular dystrophy (which includes FSHD1 and FSHD2) by administering the pharmaceutical composition to the subject.

In various embodiments, the administration may be intravenous, subcutaneous, pulmonary, intramuscular, intraperitoneal, dermal, oral, nasal, or via inhalation. In some embodiments, the administration may be once daily, weekly, every two weeks, monthly, every two months, quarterly, or yearly. In some embodiments, the administration may comprise an effective dose of from 0.01 to 100 mg/kg. In some embodiments, the administration inhibits the expression of DUX4 in the subject.

In another aspect, the invention comprises a kit comprising one or more double-stranded siRNA and a device for administering said double-stranded siRNA.

In another aspect, the present invention includes a method of ameliorating, preventing, delaying onset, or treating facioscapulohumeral muscular dystrophy (which includes FSHD1 and FSHD2) comprising administering a double-stranded small interfering RNA described herein. In another aspect, the present invention includes use of a double-stranded small interfering RNA described herein for the treatment of facioscapulohumeral muscular dystrophy.

In another aspect, the present invention includes a method of ameliorating, preventing, delaying onset, or treating cancer, comprising administering a double-stranded small interfering RNA described herein. In some embodiments, the method may further comprise the administration of a checkpoint inhibitor such as an anti-CTLA-4 agent.

In another aspect, the present invention includes use of a double-stranded small interfering RNA described herein for the preparation of a medicament for the treatment of facioscapulohumeral muscular dystrophy.

In another aspect, the present invention includes a method of inhibiting expression of DUX4 in a cell, comprising contacting a cell with a double-stranded small interfering RNA described here, and thereby inhibiting expression of DUX4. In some embodiments, the contacting is performed in vitro. In some embodiments, the contacting is performed in vivo. In some embodiments, the cell is in an animal. In some embodiments, the animal is a human. In some embodiments, the expression of DUX4 is inhibited by at least 10%, at least 20%, at least 30%, at least 40%, at least 50%, at least 60%, at least 70%, at least 80%, or at least 90%. In some embodiments, the expression of DUX4 is abolished.

### BRIEF DESCRIPTION OF THE DRAWINGS

FIG. 1A shows in vitro MBD3L2 expression data obtained with unmodified siRNA molecules targeting DUX4.

FIG. 1B shows in vitro MBD3L2 expression data obtained with modified siRNA molecules targeting DUX4.

### DETAILED DESCRIPTION OF THE INVENTION

Described herein are double-stranded small interfering RNA (siRNA) molecules that target sequences within the DUX4 gene. Also described are methods of reducing or inhibiting expression of DUX4 in a cell comprising contacting the cell with the said siRNA molecules. Further described herein are methods for the prevention or treatment of facioscapulohumeral muscular dystrophy (FSHD) by administering to a subject the double-stranded siRNA molecules and compositions comprising the same.

### Definitions

It is to be understood that both the foregoing general description and the following detailed description are exemplary and explanatory only and are not restrictive of the invention, as claimed. Herein, the use of the singular includes the plural unless specifically stated otherwise. As used herein, the use of“or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

As used herein, the term “DUX4” may refer to a DUX4 protein or a DUX4 nucleic acid, i.e., a nucleic acid sequence encoding a DUX4 protein. A DUX4 nucleic acid may refer to a DNA sequence encoding DUX4 protein, an RNA sequence transcribed from DNA encoding DUX4 (including genomic DNA comprising introns and exons) including a non-protein encoding (i.e., non-coding) RNA sequence, and an mRNA sequence encoding DUX4. In some embodiments, DUX4 nucleic acid sequence comprises GENBANK Accession No. FJ439133.1 (SEQ ID NO: 593).

“Double-stranded small interfering RNA” means any duplex RNA structure comprising two anti-parallel and substantially complementary nucleic acid strands. In certain embodiments, double-stranded small interfering RNA comprise a sense strand and an antisense strand, wherein the antisense strand is complementary to a target nucleic acid.

“Complementarity” means the capacity for pairing between nucleobases of a first nucleic acid and a second nucleic acid.

“Contiguous nucleobases” means nucleobases immediately adjacent to each other.

“Deoxyribonucleotide” means a nucleotide having a hydrogen at the 2′ position of the sugar portion of the nucleotide. Deoxyribonucleotides may be modified with any of a variety of substituents.

“Expression” includes all the functions by which a gene's coded information is converted into structures present and operating in a cell. Such structures include, but are not limited to the products of transcription and translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Inhibiting the expression or activity” refers to a reduction or blockade of the expression or activity and does not necessarily indicate a total elimination of expression or activity.

“Internucleoside linkage” refers to the chemical bond between nucleosides.

“Linked nucleosides” means adjacent nucleosides linked together by an internucleoside linkage.

“Modified internucleoside linkage” refers to a substitution or any change from a naturally occurring internucleoside bond (i.e., a phosphodiester internucleoside bond).

“Nucleobase” means a heterocyclic moiety capable of pairing with a base of another nucleic acid. “Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Nucleoside” means a nucleobase linked to a sugar. “Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

“Modified sugar” means substitution and/or any change from a natural sugar moiety. In certain embodiments modified sugars include 2′-F modified sugars and 2′-OMe modified sugars.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Sites,” as used herein, are defined as unique nucleobase positions within a target nucleic acid.

“Subject” means a human or non-human animal selected for treatment or therapy.

“Target gene” refers to a gene encoding a target.

“Target nucleic acid” refers to a nucleic acid, the modulation of which is desired.

Double-Stranded Small Interfering RNA (siRNA) Molecules

In one aspect, the present invention includes double-stranded oligonucleotides, such as small interfering RNA (siRNA) compounds, and compositions comprising the same. In some embodiments, siRNA compounds may comprise at least one modified RNA nucleoside (i.e., independently, a modified sugar moiety and/or modified nucleobase), and/or modified internucleoside linkages. In certain embodiments, siRNA compounds may comprise modified RNA nucleosides, modified DNA nucleosides, and/or modified internucleoside linkages.

Some embodiments relate to double-stranded molecules wherein each strand comprises a motif defined by the location of one or more modified or unmodified nucleosides.

In some embodiments, compositions are provided comprising a first and a second oligomeric compound that are fully or at least partially hybridized to form a duplex region and further comprising a region that is complementary to and hybridizes to a nucleic acid target. Such a composition may comprise a first oligomeric compound that is an antisense strand having full or partial complementarity to a nucleic acid target and a second oligomeric compound that is a sense strand having one or more regions of complementarity to and forming at least one duplex region with the first oligomeric compound.

In some embodiments, the compositions of the present invention modulate gene expression by hybridizing to a nucleic acid target resulting in loss of its normal function. In some embodiments, the degradation of the target nucleic acid is facilitated by an activated RISC complex that is formed with compositions of the invention.

In some embodiments, one of the strands is useful in, for example, influencing the preferential loading of the opposite strand into the RISC (or cleavage) complex. In some embodiments, the compositions of the present invention hybridize to a portion of a target RNA resulting in loss of normal function of the target RNA.

Some embodiments are drawn to double-stranded compositions wherein both the strands comprises a hemimer motif, a fully modified motif, a positionally modified motif or an alternating motif. Each strand of the compositions of the present invention may be modified to fulfill a particular role in for example the siRNA pathway. Using a different motif in each strand or the same motif with different chemical modifications in each strand permits targeting the antisense strand for the RISC complex while inhibiting the incorporation of the sense strand. Within this model, each strand may be independently modified such that it is enhanced for its particular role. The antisense strand may be modified at the 5′-end to enhance its role in one region of the RISC while the 3′-end may be modified differentially to enhance its role in a different region of the RISC.

The double-stranded oligonucleotide molecules may comprise self-complementary sense and antisense regions, wherein the antisense region comprises a nucleotide sequence that is complementary to nucleotide sequence in a target nucleic acid molecule or a portion thereof and the sense region having nucleotide sequence corresponding to the target nucleic acid sequence or a portion thereof. The double-stranded oligonucleotide molecules can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double-stranded structure, for example wherein the double-stranded region is about 12 to about 30, e.g., about 12, 13, 14, 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 12 to about 30 or more nucleotides of the double-stranded oligonucleotide molecule are complementary to the target nucleic acid or a portion thereof). Alternatively, the double-stranded oligonucleotide may be assembled from a single oligonucleotide, where the self-complementary sense and antisense regions of the siRNA are linked by means of a nucleic acid based or non-nucleic acid-based linker(s).

The double-stranded oligonucleotide may have a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof and the sense region having a nucleotide sequence corresponding to the target nucleic acid sequence or a portion thereof. The double-stranded oligonucleotide can be a circular single-stranded polynucleotide having two or more loop structures and a stem comprising self-complementary sense and antisense regions, wherein the antisense region comprises a nucleotide sequence that is complementary to a nucleotide sequence in a target nucleic acid molecule or a portion thereof and the sense region having a nucleotide sequence corresponding to the target nucleic acid sequence or a portion thereof, and wherein the circular polynucleotide can be processed either in vivo or in vitro to generate an active siRNA molecule capable of mediating RNAi.

In some embodiments, the double-stranded oligonucleotide comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In some embodiments, the double-stranded oligonucleotide comprises a nucleotide sequence that is complementary to a nucleotide sequence of a target gene. In another embodiment, the double-stranded oligonucleotide interacts with a nucleotide sequence of a target gene in a manner that causes inhibition of expression of the target gene.

As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, double-stranded oligonucleotides can be used to epigenetically silence genes at both the post-transcriptional level and the pre-transcriptional level. In a non-limiting example, epigenetic regulation of gene expression by siRNA molecules of the invention can result from siRNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

It is contemplated that compounds and compositions of some embodiments provided herein can target by a dsRNA-mediated gene silencing or RNAi mechanism, including, e.g., “hairpin” or stem-loop double-stranded RNA effector molecules in which a single RNA strand with self-complementary sequences is capable of assuming a double-stranded conformation, or duplex dsRNA effector molecules comprising two separate strands of RNA. In various embodiments, the dsRNA consists entirely of ribonucleotides or consists of a mixture of ribonucleotides and deoxynucleotides, such as the RNA/DNA hybrids disclosed, for example, by WO 00/63364, filed Apr. 19, 2000, or U.S. Ser. No. 60/130,377, filed Apr. 21, 1999.

As used herein, double-stranded oligonucleotides need not be limited to those molecules containing only RNA, but further encompasses chemically modified nucleotides and non-nucleotides. In certain embodiments, the short interfering nucleic acid molecules may lack ribonucleotides or 2′-hydroxy (2′-OH) containing nucleotides. Such double-stranded oligonucleotides that do not require the presence of ribonucleotides within the molecule to support RNAi can however have an attached linker or linkers or other attached or associated groups, moieties, or chains containing one or more nucleotides with 2′—OH groups. In some embodiments, the double-stranded oligonucleotides can comprise ribonucleotides at about 5, 10, 20, 30, 40, or 50% of the nucleotide positions.

The dsRNA or dsRNA effector molecule may be a single molecule with a region of self-complementarity such that nucleotides in one segment of the molecule base pair with nucleotides in another segment of the molecule. In various embodiments, a dsRNA that consists of a single molecule consists entirely of ribonucleotides or includes a region of ribonucleotides that is complementary to a region of deoxyribonucleotides. Alternatively, the dsRNA may include two different strands that have a region of complementarity to each other.

In various embodiments, both strands consist entirely of ribonucleotides, one strand consists entirely of ribonucleotides and one strand consists entirely of deoxyribonucleotides, or one or both strands contain a mixture of ribonucleotides and deoxyribonucleotides.

In certain embodiments, the regions of complementarity are at least 70, 80, 90, 95, 98, or 100% complementary to each other and to a target nucleic acid sequence. In certain embodiments, the region of the dsRNA that is present in a double-stranded conformation includes at least 6, 7, 8, 9, 10, 11, 12, 13, 14, 15, 16, 17, 18, 19, 20, 21, 22, 23, 24, 25, 26, 27, 28, 29, 30, 50, 75, 100, 200, 500, 1000, 2000 or 5000 nucleotides or includes all of the nucleotides in a cDNA or other target nucleic acid sequence being represented in the dsRNA. In some embodiments, the dsRNA does not contain any single stranded regions, such as single stranded ends, or the dsRNA is a hairpin. In other embodiments, the dsRNA has one or more single stranded regions or overhangs. In certain embodiments, RNA/DNA hybrids include a DNA strand or region that is an antisense strand or region (e.g, has at least 70, 80, 90, 95, 98, or 100% complementarity to a target nucleic acid) and an RNA strand or region that is a sense strand or region (e.g, has at least 70, 80, 90, 95, 98, or 100% identity to a target nucleic acid), and vice versa.

In various embodiments, the RNA/DNA hybrid is made in vitro using enzymatic or chemical synthetic methods such as those described herein or those described in WO 00/63364, filed Apr. 19, 2000, or U.S. Ser. No. 60/130,377, filed Apr. 21, 1999. In other embodiments, a DNA strand synthesized in vitro is complexed with an RNA strand made in vivo or in vitro before, after, or concurrent with the transformation of the DNA strand into the cell.

In yet other embodiments, the dsRNA is a single circular nucleic acid containing a sense and an antisense region, or the dsRNA includes a circular nucleic acid and either a second circular nucleic acid or a linear nucleic acid (see, for example, WO 00/63364, filed Apr. 19, 2000, or U.S. Ser. No. 60/130,377, filed Apr. 21, 1999). Exemplary circular nucleic acids include lariat structures in which the free 5′ phosphoryl group of a nucleotide becomes linked to the 2′ hydroxyl group of another nucleotide in a loop back fashion. Chemically synthesized RNA duplexes in the 25-30 base length range have been shown to exhibit increased potency compared with shorter 21-mer siRNAs and the increase in potency is attributed to the action of Dicer endonuclease enzyme which uses the longer dsRNA as substrate, cleaves it and facilitates the loading of the cleaved dsRNA into the RISC. Thus, in some embodiments, the dsRNA may be a dicer substrate RNA. For example, in some embodiments, the dsRNA is a 25/27 mer.

Modifled Nucleotides and Chemically Modified siRNAs

In various embodiments described herein, a double-stranded siRNA of the invention may comprise one or more (e.g., two, three, four, five, or more) modified nucleic acid monomers (i.e., nucleotides). Various examples of modified nucleotides are disclosed in U.S. Pat. Nos. 9,035,039, 9,951,338, 10,036,024, 10,538,763, and International Patent Publication No. WO/2018/222926, each of which is herein incorporated by reference in its entirety.

Examples of modified nucleotides suitable for use in the present invention include, but are not limited to, ribonucleotides or arabinonucleotides having a 2′-O-methyl (2′-OMe), 2′-deoxy-2′-fluoro (2′-F), 2′-deoxy, 5-C-methyl, 2′-O-(2-methoxyethyl) (MOE), 4′-thio, 2′-amino, or a 2′-C-allyl group. In some embodiments, these may include 2′-deoxy-2′-fluoro arabinoguanosine nucleotides.

Modified nucleotides having a conformation such as those described in the art, for example in Saenger, Principles of Nucleic Acid Structure, Springer-Verlag Ed. (1984), are also suitable for use in siRNA molecules. Other modified nucleotides include, without limitation, locked nucleic acid (LNA) nucleotides, unlocked nucleic acid (UNA) nucleotides, G-clamp nucleotides, or nucleotide base analogs. LNA nucleotides include but need not be limited to 2′-O, 4′-C-methylene-(D-ribofuranosyl)nucleotides), 2′-O-(2-methoxyethyl) (MOE) nucleotides, 2′-methyl-thio-ethyl nucleotides, 2′-deoxy-2′-fluoro (2′-F) nucleotides, 2′-deoxy-2′-chloro (2′-Cl) nucleotides, 2′-azido nucleotides, and (S)-constrained ethyl (cEt) nucleotides.

In some embodiments, a double-stranded siRNA molecule may comprise one or more chemical modifications such as terminal cap moieties, phosphate backbone modifications, and the like. Examples of classes of terminal cap moieties include, without limitation, inverted deoxy abasic residues, glyceryl modifications, 4′,5′-methylene nucleotides, 1-(beta-D-erythrofuranosyl) nucleotides, 4′-thio nucleotides, carbocyclic nucleotides, 1,5-anhydrohexitol nucleotides, L-nucleotides, alpha-nucleotides, modified base nucleotides, threo pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3 aminopropyl phosphate, 6-aminohexyl phosphate, 1,2-aminododecyl phosphate, hydroxypropyl phosphate, 1,4-butanediol phosphate, 3′-phosphoramidate, 5′ phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties. Non-limiting examples of phosphate backbone modifications (i.e., resulting in modified internucleoside linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetyl, thioformacetal, and alkylsilyl substitutions. Such chemical modifications can occur at the 5′-end and/or 3′-end of the sense strand, antisense strand, or both strands of the siRNA.

In some embodiments, a double-stranded siRNA of the invention may comprise at least one modified internucleoside linkage. Examples of modified internucleoside linkage include, without limitation, peptide linkage, phosphorothioate (PS) linkage, and phosphorodiamidate morpholino (PMO) linkage. In some embodiments, a double-stranded siRNA of the invention comprises 2, 3, 4, 5, or more modified internucleoside linkages. In some embodiments, a double-stranded siRNA of the invention comprises a sense strand, an antisense strand, or both, where all internucleoside linkages are modified. In some embodiments, each internucleoside linkage is a phosphorothioate internucleoside linkage.

In some embodiments, the sense and/or antisense strand may comprise a 5′-terminal or 3′-terminal overhang having 1, 2, 3, 4, or more 2′-deoxyribonucleotides (e.g., A, G, C, or T) and/or any combination of modified and unmodified nucleotides. In some embodiments, a double-stranded siRNA may comprise a TT overhang at the 3′ end of the sense strand. In some embodiments, a double-stranded siRNA may comprise a TT overhang at the 3′ end of the antisense strand. In some exemplary embodiments, a double-stranded siRNA may comprise a TT overhang at the 3′ end of sense strand and at the 3′ end of the antisense strand.

Conjugated siRNAs

In some embodiments, a double-stranded siRNA of the invention may be conjugated to at least one other molecule. Conjugation of the double-stranded siRNA with an appropriate molecule provides a means for improving delivery of the siRNA into target cells. Such conjugate molecules may interact with the lipid components of the cell membrane, bind to specific cell surface proteins or receptors, and/or penetrate the cell through endogenous transport mechanisms carrying the siRNA with them.

The conjugate can be attached at the 5′- and/or the 3′-end of the sense and/or the antisense strand of the siRNA via a covalent attachment such as a nucleic acid or non-nucleic acid linker. The conjugate can be attached to the siRNA through a carbamate group or other linking group (see, e.g., U.S. Patent Publication Nos. 20050074771, 20050043219, and 20050158727). A conjugate may be added to siRNA for any of a number of purposes. For example, the conjugate may be a molecular entity that facilitates the delivery of siRNA into a cell or may be a molecule that comprises a drug or label. Examples of conjugate molecules suitable for attachment to siRNA of the present invention include, without limitation, lipophilic molecules (e.g., fatty acids), cholesterols, glycols such as polyethylene glycol (PEG), human serum albumin (HSA), carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, antibodies, aptamers, and combinations thereof (see, e.g., U.S. Patent Publication Nos. 20030130186, 20040110296, and 20040249178; U.S. Pat. No. 6,753,423).

The type of conjugate used and the extent of conjugation to the siRNA can be evaluated for improved pharmacokinetic profiles, bioavailability, and/or stability of the siRNA while retaining activity. As such, one skilled in the art can screen siRNA molecules having various conjugates attached thereto to identify siRNA conjugates having improved properties using any of a variety of well-known in vitro cell culture or in vivo animal models including the negative-controlled expression studies described above. Examples of siRNA bioconjugates are described in, e.g., Chernikov et al., 2019, Front. Pharmacol. 10: 1-25 and Osborn et al., 2018, Nuc. Acid Ther. 28(3): 128-136.

In some embodiments, a double-stranded siRNA of the invention may be conjugated to a lipophilic molecule (for example, a long chain fatty acid or LCFA), an antibody (for example, anti-transferrin receptor antibody), an aptamer, a ligand, a peptide, or a polymer.

In one embodiment, the double-stranded siRNA may be conjugated to a lipophilic molecule, e.g., a long chain fatty acid. In some exemplary embodiments, a double-stranded siRNA of the invention is conjugated to a long chain fatty acid described in International Patent Publication No. WO/2019/232255.

In some embodiments, a double-stranded siRNA of the invention may be conjugated to an antibody. In some embodiments, the antibody is a muscle-targeting antibody. In some embodiments, the muscle-targeting antibody is an anti-transferrin receptor antibody. In some exemplary embodiments, a double-stranded siRNA of the invention is conjugated to an anti-transferrin receptor antibody described in International Patent Publication No. WO/2020/028864.

siRNA Delivery with Liposomes, Lipid Nanoparticles (LNPs), and Other Carriers

In some embodiments, a double-stranded siRNA of the invention may be delivered via liposomes, nanoparticles, lipid nanoparticles (LNPs), polymers, microparticles, microcapsules, micelles, or extracellular vesicles.

In some embodiments, a double-stranded siRNA of the invention is delivered via a lipid nanoparticle (LNP). Examples of LNPs capable of delivering a double-stranded siRNA of the invention are described in International Patent Publication Nos. WO/2015/074085, WO/2016/081029, WO/2017/117530, WO/2018/118102, WO/2018/119163, WO/2018/222926, WO/2019/191780, and WO/2020/154746. In some embodiments, an LNP may be decorated with targeting moiety, e.g., an antibody, a receptor, or a fragment thereof capable of binding to a target ligand.

In one embodiment, a lipid nanoparticle for use in the instant invention comprises (a) a nucleic acid (e.g., a double-stranded siRNA), (b) a cationic lipid, (c) an aggregation reducing agent (such as a PEG-lipid), (d) optionally a non-cationic lipid (such as a neutral lipid), and (e) optionally a sterol. In one embodiment, the lipid nanoparticle comprises (i) at least one cationic lipid; (ii) a neutral lipid, e.g., DSPC; (iii) a sterol, e.g., cholesterol; and (iv) a PEG-lipid, in a molar ratio of about 20-65% cationic lipid: 5-25% neutral lipid: 25-55% sterol; 0.5-15% PEG-lipid. In some embodiments, the cationic lipid is selected from ATX-002, ATX-081, ATX-095, or ATX-126, as described in WO/2018/222926.

In some embodiments, a double-stranded siRNA of the invention is delivered via a nanocarrier comprising a molecule enabling specific receptor-mediated endosomal uptake. In one embodiment, said molecule can enable receptor binding, endosomal uptake, controlled breakdown of the endosomal membrane, and release of siRNA into a target cell. Examples of nanocarriers capable of delivering a double-stranded siRNA of the invention are described in WO/2009/141257. In some embodiments, the nanocarrier is a lipid-based nanocarrier, e.g., a lipid nanoparticle (LNP).

### DUX4

In another aspect, the present invention includes a method of reducing expression of DUX4 in a cell comprising contacting the cell with a double-stranded small interfering RNA compound targeted to DUX4. In certain embodiments, DUX4 comprises a nucleic acid sequence at least 85% identical to SEQ ID NO: 593. In certain embodiments, DUX4 comprises a nucleic acid sequence at least 85% complementary to SEQ ID NO: 593.

The inefficient epigenetic repression of DUX4 in skeletal muscle leads to aberrant expression of the DUX4 protein and facioscapulohumeral muscular dystrophy (FSHD) 1 and 2. FSHD1 and 2 patients exhibit progressive, asymmetric muscle weakness. Therefore, in certain embodiments it is desirable to inhibit expression of DUX4. In certain embodiments it is desirable to inhibit expression of DUX4 in a subject having 10 or fewer D4Z4 repeats.

In certain embodiments, DUX4 expression is inhibited by contacting a cell with a double-stranded small interfering RNA compound. In certain embodiments, DUX4 expression is inhibited by contacting a cell with a double-stranded small interfering RNA compound disclosed herein.

### Pharmaceutical Compositions

In certain embodiments, the present invention provides pharmaceutical compositions comprising one or more the double-stranded small interfering RNA compounds. In certain embodiments, such pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one or more antisense compound and sterile water. In certain embodiments, the sterile saline is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile phosphate-buffered saline (PBS). In certain embodiments, the sterile saline is pharmaceutical grade PBS.

In certain embodiments, the double-stranded small interfering RNA compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising the double-stranded small interfering RNA compounds encompass any pharmaceutically acceptable salts, esters, or salts of such esters. In certain embodiments, pharmaceutical compositions comprising the double-stranded small interfering RNA compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of the double-stranded small interfering RNA compounds, prodrugs, pharmaceutically acceptable salts of such prodrugs, and other bioequivalents. Suitable pharmaceutically acceptable salts include, but are not limited to, sodium and potassium salts.

In certain embodiments, one or more the double-stranded small interfering RNA compounds provided herein is formulated as a prodrug. A prodrug can include the incorporation of additional nucleosides at one or both ends of a double-stranded small interfering RNA compound which are cleaved by endogenous nucleases within the body, to form the active antisense oligomeric compound. In certain embodiments, upon in vivo administration, a prodrug is chemically or enzymatically converted to the biologically, pharmaceutically or therapeutically more active form of an oligonucleotide. In certain embodiments, prodrugs are useful because they are easier to administer than the corresponding active form or to be processed by RISC. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility.

In certain embodiments, pharmaceutical compositions provided herein comprise one or more the double-stranded small interfering RNA compounds and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

### Administration and Dosages

The siRNA molecules and the compositions comprising them may be administered to a subject by any suitable route. For example, the administration may be intravenous, subcutaneous, pulmonary, intramuscular, intraperitoneal, dermal, oral, nasal, or via inhalation.

For example, in certain embodiments, a pharmaceutical composition provided herein is prepared for oral administration. In certain embodiments, a pharmaceutical composition is prepared for administration by injection (e.g., intravenous, subcutaneous, intramuscular, dermal, intraperitoneal etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, a pharmaceutical composition is prepared for transmucosal administration. In some embodiments, penetrants appropriate to the barrier to be permeated are used in the formulation. Such penetrants are generally known in the art. In certain embodiments, a pharmaceutical composition is prepared for pulmonary delivery, e.g. intratracheal, intranasal or via inhalation. Compositions suitable for intranasal preparation can be administered into the nasal cavity as nasal suspensions. Compositions suitable for inhalation may be provided as pharmaceutical aerosols, for example, solution aerosols or powder aerosols, and may be administered using devices such as inhalers, e.g. metered dose inhalers (MDIs) or dry powder inhalers (DPIs), and nebulizers. By controlling the particle characteristics and deposition mechanics, or by specifically targeting certain cell types in the lung, for example by coupling ligands that bind to receptors expressed on the surface of target cells, compositions can be delivered to specific regions in the pulmonary tract. In certain embodiments, a pharmaceutical composition is prepared for intranasal administration. In certain embodiments it is administered via inhalation.

In certain embodiments, a pharmaceutical composition provided herein comprises the double-stranded small interfering RNA in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art. In some embodiments, the administration comprises an effective dose of from 0.01 to 100 mg/kg. The administration may be once daily, weekly, every two weeks, monthly, every two months, or quarterly.

In certain embodiments, the present invention provides compositions and methods for reducing the amount or activity of a target nucleic acid in a cell. In certain embodiments, the cell is in an animal. In certain embodiments, the animal is a mammal. In certain embodiments, the animal is a rodent. In certain embodiments, the animal is a primate. In certain embodiments, the animal is a non-human primate. In certain embodiments, the animal is a human.

In certain embodiments, the present invention provides methods of administering a pharmaceutical composition comprising a double-stranded small interfering RNA compound of the present disclosure to an animal. Suitable administration routes include, but are not limited to, oral, rectal, transmucosal, intestinal, enteral, topical, suppository, through inhalation, intrathecal, intracerebroventricular, intraperitoneal, intranasal, intratumoral, and parenteral (e.g., intravenous, intramuscular, intramedullary, and subcutaneous). In certain embodiments, pharmaceutical intrathecals are administered to achieve local rather than systemic exposures. For example, pharmaceutical compositions may be injected directly in the area of desired effect (e.g., into the ears).

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine(methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” (SEQ ID NO:689) encompasses any oligomeric compounds having such nucleobase sequence, whether modified or unmodified, including, but not limited to, such compounds comprising RNA bases, such as those having sequence “AUCGAUCG” (SEQ ID NO: 690) and those having some DNA bases and some RNA bases such as “AUCGATCG” (SEQ ID NO: 691) and oligomeric compounds having other modified or naturally occurring bases, such as “ATmeCGAUCG,” (SEQ ID NO: 692) wherein meC indicates a cytosine base comprising a methyl group at the 5-position.

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

It is understood that the sequence set forth in each SEQ ID NO described herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase.

### EXAMPLES

### Example 1: Design and Sequences of siRNA Targeting the DUX4 Coding and Upstream D4Z4 Repeat Region

Double-stranded small interfering RNA (siRNA) targeting the human DUX4 promoter region and coding region were designed in silico. The sequences are listed below in Table 1. Each siRNA listed in Table 1 is targeted against the human genomic DUX4 sequence (GENBANK Accession No. FJ439133.1, SEQ ID NO: 593). “Start” indicates the 5-most nucleoside to which the siRNA is targeted in the genomic DUX4 sequence. “Stop” indicates the 3-most nucleoside to which the siRNA is targeted in the genomic DUX4 sequence.

TABLE 1
Double-Stranded siRNA Targeting Human DUX4

| | SEQ | | SEQ | | | |
|---|---|---|---|---|---|---|
| | ID | | ID | | Start | Stop |
| Oligo name | NO. | sense | NO: | antisense | site | site |
| UGNX394 | 1 | GGCAGAGAUGGAGAGAGGA | 2 | UCCUCUCUCCAUCUCUGCC | 4605 | 4623 |
| UGNX482 | 3 | AGGGAGGAACGGAGGGAAA | 4 | UUUCCCUCCGUUCCUCCCU | 4693 | 4711 |
| UGNX554 | 5 | GAGGAAGGCAGGGAGGAAA | 6 | UUUCCUCCCUGCCUUCCUC | 4765 | 4783 |
| UGNX722 | 7 | CGGUUUCCUCCGGGACAAA | 8 | UUUGUCCCGGAGGAAACCG | 4933 | 4951 |
| UGNX779 | 9 | CGGUUCACAGACCGCACAU | 10 | AUGUGCGGUCUGUGAACCG | 4990 | 5008 |
| UGNX864 | 11 | GACGACGGAGGCGUGAUUU | 12 | AAAUCACGCCUCCGUCGUC | 5075 | 5093 |
| UGNX916 | 13 | GCCUGUUGCUCACGUCUCU | 14 | AGAGACGUGAGCAACAGGC | 5127 | 5145 |
| UGNX950 | 15 | CUGGCCAUGCCGACUGUUU | 16 | AAACAGUCGGCAUGGCCAG | 5161 | 5179 |
| UGNX991 | 17 | CCGGAAACAUGCAGGGAAG | 18 | CUUCCCUGCAUGUUUCCGG | 5202 | 5220 |
| UGNX1032 | 19 | UUCGCUCUCCUUGCCAGGU | 20 | ACCUGGCAAGGAGAGCGAA | 5243 | 5261 |
| UGNX1094 | 21 | GGAAUCCAUCGUCAGGCCA | 22 | UGGCCUGACGAUGGAUUCC | 5305 | 5323 |
| UGNX1142 | 23 | GUCUCGCUCUGGUCUUCUA | 24 | UAGAAGACCAGAGCGAGAC | 5353 | 5371 |
| UGNX1223 | 25 | CACAGGCAUUGCCUCCUUC | 26 | GAAGGAGGCAAUGCCUGUG | 5434 | 5452 |
| UGNX1253 | 27 | GCCUGGCACACUCAAGACU | 28 | AGUCUUGAGUGUGCCAGGC | 5464 | 5482 |
| UGNX1311 | 29 | AGGCUGGUUUCUCCCUGCU | 30 | AGCAGGGAGAAACCAGCCU | 5522 | 5540 |
| UGNX1440 | 31 | CUUCCUCUUCGUCUCUCCG | 32 | CGGAGAGACGAAGAGGAAG | 5651 | 5669 |
| UGNX1599 | 33 | CUCACCGCCAUUCAUGAAG | 34 | CUUCAUGAAUGGCGGUGAG | 5810 | 5828 |
| UGNX1631 | 35 | CUGCCUGUGGGCCUUUACA | 36 | UGUAAAGGCCCACAGGCAG | 5842 | 5860 |
| UGNX1758 | 37 | GCGUCCGUCCGUGAAAUUC | 38 | GAAUUUCACGGACGGACGC | 5969 | 5987 |
| UGNX1855 | 39 | CGACGGAGACUCGUUUGGA | 40 | UCCAAACGAGUCUCCGUCG | 6066 | 6084 |
| UGNX1898 | 41 | GAGCCUGCUUUGAGCGGAA | 42 | UUCCGCUCAAAGCAGGCUC | 6109 | 6127 |
| UGNX1972 | 43 | GAGCCCAGGGUCCAGAUUU | 44 | AAAUCUGGACCCUGGGCUC | 6183 | 6201 |
| UGNX2119 | 45 | CUCCUCCGAGCCUUUGAGA | 46 | UCUCAAAGGCUCGGAGGAG | 6330 | 6348 |
| UGNX2192 | 47 | UCCCGGAGUCCAGGAUUCA | 48 | UGAAUCCUGGACUCCGGGA | 6403 | 6421 |
| UGNX2909 | 49 | UGCUGCUGGAUGAGCUCCU | 50 | AGGAGCUCAUCCAGCAGCA | 7120 | 7138 |
| UGNX2952 | 51 | GGCGCAACCUCUCCUAGAA | 52 | UUCUAGGAGAGGUUGCGCC | 7163 | 7181 |
| UGNX3028 | 53 | CUCAGCGAGGAAGAAUACC | 54 | GGUAUUCUUCCUCGCUGAG | 7239 | 7257 |
| UGNX3194 | 55 | CUGGGAUUCCUGCCUUCUA | 56 | UAGAAGGCAGGAAUCCCAG | 7405 | 7423 |
| UGNX3242 | 57 | GCGGAGAACUGCCAUUCUU | 58 | AAGAAUGGCAGUUCUCCGC | 7453 | 7471 |
| UGNX395 | 59 | GCAGAGAUGGAGAGAGGAA | 60 | UUCCUCUCUCCAUCUCUGC | 4606 | 4624 |
| UGNX396 | 61 | CAGAGAUGGAGAGAGGAAC | 62 | GUUCCUCUCUCCAUCUCUG | 4607 | 4625 |
| UGNX397 | 63 | AGAGAUGGAGAGAGGAACG | 64 | CGUUCCUCUCUCCAUCUCU | 4608 | 4626 |
| UGNX398 | 65 | GAGAUGGAGAGAGGAACGG | 66 | CCGUUCCUCUCUCCAUCUC | 4609 | 4627 |
| UGNX399 | 67 | AGAUGGAGAGAGGAACGGG | 68 | CCCGUUCCUCUCUCCAUCU | 4610 | 4628 |
| UGNX400 | 69 | GAUGGAGAGAGGAACGGGA | 70 | UCCCGUUCCUCUCUCCAUC | 4611 | 4629 |
| UGNX401 | 71 | AUGGAGAGAGGAACGGGAG | 72 | CUCCCGUUCCUCUCUCCAU | 4612 | 4630 |
| UGNX402 | 73 | UGGAGAGAGGAACGGGAGA | 74 | UCUCCCGUUCCUCUCUCCA | 4613 | 4631 |
| UGNX405 | 75 | AGAGAGGAACGGGAGACCU | 76 | AGGUCUCCCGUUCCUCUCU | 4616 | 4634 |
| UGNX406 | 77 | GAGAGGAACGGGAGACCUA | 78 | UAGGUCUCCCGUUCCUCUC | 4617 | 4635 |
| UGNX407 | 79 | AGAGGAACGGGAGACCUAG | 80 | CUAGGUCUCCCGUUCCUCU | 4618 | 4636 |
| UGNX408 | 81 | GAGGAACGGGAGACCUAGA | 82 | UCUAGGUCUCCCGUUCCUC | 4619 | 4637 |
| UGNX409 | 83 | AGGAACGGGAGACCUAGAG | 84 | CUCUAGGUCUCCCGUUCCU | 4620 | 4638 |
| UGNX484 | 85 | GGAGGAACGGAGGGAAAGA | 86 | UCUUUCCCUCCGUUCCUCC | 4695 | 4713 |
| UGNX485 | 87 | GAGGAACGGAGGGAAAGAC | 88 | GUCUUUCCCUCCGUUCCUC | 4696 | 4714 |
| UGNX486 | 89 | AGGAACGGAGGGAAAGACA | 90 | UGUCUUUCCCUCCGUUCCU | 4697 | 4715 |
| UGNX487 | 91 | GGAACGGAGGGAAAGACAG | 92 | CUGUCUUUCCCUCCGUUCC | 4698 | 4716 |
| UGNX488 | 93 | GAACGGAGGGAAAGACAGA | 94 | UCUGUCUUUCCCUCCGUUC | 4699 | 4717 |
| UGNX489 | 95 | AACGGAGGGAAAGACAGAG | 96 | CUCUGUCUUUCCCUCCGUU | 4700 | 4718 |
| UGNX490 | 97 | ACGGAGGGAAAGACAGAGC | 98 | GCUCUGUCUUUCCCUCCGU | 4701 | 4719 |
| UGNX492 | 99 | GGAGGGAAAGACAGAGCGA | 100 | UCGCUCUGUCUUUCCCUCC | 4703 | 4721 |
| UGNX493 | 101 | GAGGGAAAGACAGAGCGAC | 102 | GUCGCUCUGUCUUUCCCUC | 4704 | 4722 |
| UGNX494 | 103 | AGGGAAAGACAGAGCGACG | 104 | CGUCGCUCUGUCUUUCCCU | 4705 | 4723 |
| UGNX496 | 105 | GGAAAGACAGAGCGACGCA | 106 | UGCGUCGCUCUGUCUUUCC | 4707 | 4725 |
| UGNX497 | 107 | GAAAGACAGAGCGACGCAG | 108 | CUGCGUCGCUCUGUCUUUC | 4708 | 4726 |
| UGNX498 | 109 | AAAGACAGAGCGACGCAGG | 110 | CCUGCGUCGCUCUGUCUUU | 4709 | 4727 |
| UGNX780 | 111 | GGUUCACAGACCGCACAUC | 112 | GAUGUGCGGUCUGUGAACC | 4991 | 5009 |
| UGNX781 | 113 | GUUCACAGACCGCACAUCC | 114 | GGAUGUGCGGUCUGUGAAC | 4992 | 5010 |
| UGNX782 | 115 | UUCACAGACCGCACAUCCC | 116 | GGGAUGUGCGGUCUGUGAA | 4993 | 5011 |
| UGNX917 | 117 | CCUGUUGCUCACGUCUCUC | 118 | GAGAGACGUGAGCAACAGG | 5128 | 5146 |
| UGNX918 | 119 | CUGUUGCUCACGUCUCUCC | 120 | GGAGAGACGUGAGCAACAG | 5129 | 5147 |
| UGNX919 | 121 | UGUUGCUCACGUCUCUCCG | 122 | CGGAGAGACGUGAGCAACA | 5130 | 5148 |
| UGNX954 | 123 | CCAUGCCGACUGUUUGCUC | 124 | GAGCAAACAGUCGGCAUGG | 5165 | 5183 |
| UGNX955 | 125 | CAUGCCGACUGUUUGCUCC | 126 | GGAGCAAACAGUCGGCAUG | 5166 | 5184 |
| UGNX956 | 127 | AUGCCGACUGUUUGCUCCC | 128 | GGGAGCAAACAGUCGGCAU | 5167 | 5185 |
| UGNX964 | 129 | UGUUUGCUCCCGGAGCUCU | 130 | AGAGCUCCGGGAGCAAACA | 5175 | 5193 |
| UGNX990 | 131 | CCCGGAAACAUGCAGGGAA | 132 | UUCCCUGCAUGUUUCCGGG | 5201 | 5219 |
| UGNX992 | 133 | CGGAAACAUGCAGGGAAGG | 134 | CCUUCCCUGCAUGUUUCCG | 5203 | 5221 |
| UGNX993 | 135 | GGAAACAUGCAGGGAAGGG | 136 | CCCUUCCCUGCAUGUUUCC | 5204 | 5222 |
| UGNX994 | 137 | GAAACAUGCAGGGAAGGGU | 138 | ACCCUUCCCUGCAUGUUUC | 5205 | 5223 |
| UGNX995 | 139 | AAACAUGCAGGGAAGGGUG | 140 | CACCCUUCCCUGCAUGUUU | 5206 | 5224 |
| UGNX996 | 141 | AACAUGCAGGGAAGGGUGC | 142 | GCACCCUUCCCUGCAUGUU | 5207 | 5225 |
| UGNX998 | 143 | CAUGCAGGGAAGGGUGCAA | 144 | UUGCACCCUUCCCUGCAUG | 5209 | 5227 |
| UGNX999 | 145 | AUGCAGGGAAGGGUGCAAG | 146 | CUUGCACCCUUCCCUGCAU | 5210 | 5228 |
| UGNX1033 | 147 | UCGCUCUCCUUGCCAGGUU | 148 | AACCUGGCAAGGAGAGCGA | 5244 | 5262 |
| UGNX1036 | 149 | CUCUCCUUGCCAGGUUCCA | 150 | UGGAACCUGGCAAGGAGAG | 5247 | 5265 |
| UGNX1037 | 151 | UCUCCUUGCCAGGUUCCAA | 152 | UUGGAACCUGGCAAGGAGA | 5248 | 5266 |
| UGNX1038 | 153 | CUCCUUGCCAGGUUCCAAA | 154 | UUUGGAACCUGGCAAGGAG | 5249 | 5267 |
| UGNX1039 | 155 | UCCUUGCCAGGUUCCAAAC | 156 | GUUUGGAACCUGGCAAGGA | 5250 | 5268 |
| UGNX1040 | 157 | CCUUGCCAGGUUCCAAACC | 158 | GGUUUGGAACCUGGCAAGG | 5251 | 5269 |
| UGNX1041 | 159 | CUUGCCAGGUUCCAAACCG | 160 | CGGUUUGGAACCUGGCAAG | 5252 | 5270 |
| UGNX1042 | 161 | UUGCCAGGUUCCAAACCGG | 162 | CCGGUUUGGAACCUGGCAA | 5253 | 5271 |
| UGNX1049 | 163 | GUUCCAAACCGGCCACACU | 164 | AGUGUGGCCGGUUUGGAAC | 5260 | 5278 |
| UGNX1050 | 165 | UUCCAAACCGGCCACACUG | 166 | CAGUGUGGCCGGUUUGGAA | 5261 | 5279 |
| UGNX1095 | 167 | GAAUCCAUCGUCAGGCCAU | 168 | AUGGCCUGACGAUGGAUUC | 5306 | 5324 |
| UGNX1096 | 169 | AAUCCAUCGUCAGGCCAUC | 170 | GAUGGCCUGACGAUGGAUU | 5307 | 5325 |
| UGNX1097 | 171 | AUCCAUCGUCAGGCCAUCA | 172 | UGAUGGCCUGACGAUGGAU | 5308 | 5326 |
| UGNX1098 | 173 | UCCAUCGUCAGGCCAUCAC | 174 | GUGAUGGCCUGACGAUGGA | 5309 | 5327 |
| UGNX1143 | 175 | UCUCGCUCUGGUCUUCUAC | 176 | GUAGAAGACCAGAGCGAGA | 5354 | 5372 |
| UGNX1144 | 177 | CUCGCUCUGGUCUUCUACG | 178 | CGUAGAAGACCAGAGCGAG | 5355 | 5373 |
| UGNX1145 | 179 | UCGCUCUGGUCUUCUACGU | 180 | ACGUAGAAGACCAGAGCGA | 5356 | 5374 |
| UGNX1146 | 181 | CGCUCUGGUCUUCUACGUG | 182 | CACGUAGAAGACCAGAGCG | 5357 | 5375 |
| UGNX1147 | 183 | GCUCUGGUCUUCUACGUGG | 184 | CCACGUAGAAGACCAGAGC | 5358 | 5376 |
| UGNX1148 | 185 | CUCUGGUCUUCUACGUGGA | 186 | UCCACGUAGAAGACCAGAG | 5359 | 5377 |
| UGNX1149 | 187 | UCUGGUCUUCUACGUGGAA | 188 | UUCCACGUAGAAGACCAGA | 5360 | 5378 |
| UGNX1150 | 189 | CUGGUCUUCUACGUGGAAA | 190 | UUUCCACGUAGAAGACCAG | 5361 | 5379 |
| UGNX1151 | 191 | UGGUCUUCUACGUGGAAAU | 192 | AUUUCCACGUAGAAGACCA | 5362 | 5380 |
| UGNX1152 | 193 | GGUCUUCUACGUGGAAAUG | 194 | CAUUUCCACGUAGAAGACC | 5363 | 5381 |
| UGNX1153 | 195 | GUCUUCUACGUGGAAAUGA | 196 | UCAUUUCCACGUAGAAGAC | 5364 | 5382 |
| UGNX1154 | 197 | UCUUCUACGUGGAAAUGAA | 198 | UUCAUUUCCACGUAGAAGA | 5365 | 5383 |
| UGNX1155 | 199 | CUUCUACGUGGAAAUGAAC | 200 | GUUCAUUUCCACGUAGAAG | 5366 | 5384 |
| UGNX1156 | 201 | UUCUACGUGGAAAUGAACG | 202 | CGUUCAUUUCCACGUAGAA | 5367 | 5385 |
| UGNX1157 | 203 | UCUACGUGGAAAUGAACGA | 204 | UCGUUCAUUUCCACGUAGA | 5368 | 5386 |
| UGNX1158 | 205 | CUACGUGGAAAUGAACGAG | 206 | CUCGUUCAUUUCCACGUAG | 5369 | 5387 |
| UGNX1159 | 207 | UACGUGGAAAUGAACGAGA | 208 | UCUCGUUCAUUUCCACGUA | 5370 | 5388 |
| UGNX1160 | 209 | ACGUGGAAAUGAACGAGAG | 210 | CUCUCGUUCAUUUCCACGU | 5371 | 5389 |
| UGNX1161 | 211 | CGUGGAAAUGAACGAGAGC | 212 | GCUCUCGUUCAUUUCCACG | 5372 | 5390 |
| UGNX1162 | 213 | GUGGAAAUGAACGAGAGCC | 214 | GGCUCUCGUUCAUUUCCAC | 5373 | 5391 |
| UGNX1163 | 215 | UGGAAAUGAACGAGAGCCA | 216 | UGGCUCUCGUUCAUUUCCA | 5374 | 5392 |
| UGNX1164 | 217 | GGAAAUGAACGAGAGCCAC | 218 | GUGGCUCUCGUUCAUUUCC | 5375 | 5393 |
| UGNX1165 | 219 | GAAAUGAACGAGAGCCACA | 220 | UGUGGCUCUCGUUCAUUUC | 5376 | 5394 |
| UGNX1166 | 221 | AAAUGAACGAGAGCCACAC | 222 | GUGUGGCUCUCGUUCAUUU | 5377 | 5395 |
| UGNX1167 | 223 | AAUGAACGAGAGCCACACG | 224 | CGUGUGGCUCUCGUUCAUU | 5378 | 5396 |
| UGNX1168 | 225 | AUGAACGAGAGCCACACGC | 226 | GCGUGUGGCUCUCGUUCAU | 5379 | 5397 |
| UGNX1222 | 227 | CCACAGGCAUUGCCUCCUU | 228 | AAGGAGGCAAUGCCUGUGG | 5433 | 5451 |
| UGNX1224 | 229 | ACAGGCAUUGCCUCCUUCA | 230 | UGAAGGAGGCAAUGCCUGU | 5435 | 5453 |
| UGNX1225 | 231 | CAGGCAUUGCCUCCUUCAC | 232 | GUGAAGGAGGCAAUGCCUG | 5436 | 5454 |
| UGNX1226 | 233 | AGGCAUUGCCUCCUUCACG | 234 | CGUGAAGGAGGCAAUGCCU | 5437 | 5455 |
| UGNX1228 | 235 | GCAUUGCCUCCUUCACGGA | 236 | UCCGUGAAGGAGGCAAUGC | 5439 | 5457 |
| UGNX1229 | 237 | CAUUGCCUCCUUCACGGAG | 238 | CUCCGUGAAGGAGGCAAUG | 5440 | 5458 |
| UGNX1230 | 239 | AUUGCCUCCUUCACGGAGA | 240 | UCUCCGUGAAGGAGGCAAU | 5441 | 5459 |
| UGNX1231 | 241 | UUGCCUCCUUCACGGAGAG | 242 | CUCUCCGUGAAGGAGGCAA | 5442 | 5460 |
| UGNX1232 | 243 | UGCCUCCUUCACGGAGAGA | 244 | UCUCUCCGUGAAGGAGGCA | 5443 | 5461 |
| UGNX1254 | 245 | CCUGGCACACUCAAGACUC | 246 | GAGUCUUGAGUGUGCCAGG | 5465 | 5483 |
| UGNX1255 | 247 | CUGGCACACUCAAGACUCC | 248 | GGAGUCUUGAGUGUGCCAG | 5466 | 5484 |
| UGNX1256 | 249 | UGGCACACUCAAGACUCCC | 250 | GGGAGUCUUGAGUGUGCCA | 5467 | 5485 |
| UGNX1257 | 251 | GGCACACUCAAGACUCCCA | 252 | UGGGAGUCUUGAGUGUGCC | 5468 | 5486 |
| UGNX1258 | 253 | GCACACUCAAGACUCCCAC | 254 | GUGGGAGUCUUGAGUGUGC | 5469 | 5487 |
| UGNX1259 | 255 | CACACUCAAGACUCCCACG | 256 | CGUGGGAGUCUUGAGUGUG | 5470 | 5488 |
| UGNX1260 | 257 | ACACUCAAGACUCCCACGG | 258 | CCGUGGGAGUCUUGAGUGU | 5471 | 5489 |
| UGNX1261 | 259 | CACUCAAGACUCCCACGGA | 260 | UCCGUGGGAGUCUUGAGUG | 5472 | 5490 |
| UGNX1262 | 261 | ACUCAAGACUCCCACGGAG | 262 | CUCCGUGGGAGUCUUGAGU | 5473 | 5491 |
| UGNX1264 | 263 | UCAAGACUCCCACGGAGGU | 264 | ACCUCCGUGGGAGUCUUGA | 5475 | 5493 |
| UGNX1265 | 265 | CAAGACUCCCACGGAGGUU | 266 | AACCUCCGUGGGAGUCUUG | 5476 | 5494 |
| UGNX1266 | 267 | AAGACUCCCACGGAGGUUC | 268 | GAACCUCCGUGGGAGUCUU | 5477 | 5495 |
| UGNX1267 | 269 | AGACUCCCACGGAGGUUCA | 270 | UGAACCUCCGUGGGAGUCU | 5478 | 5496 |
| UGNX1269 | 271 | ACUCCCACGGAGGUUCAGU | 272 | ACUGAACCUCCGUGGGAGU | 5480 | 5498 |
| UGNX1270 | 273 | CUCCCACGGAGGUUCAGUU | 274 | AACUGAACCUCCGUGGGAG | 5481 | 5499 |
| UGNX1271 | 275 | UCCCACGGAGGUUCAGUUC | 276 | GAACUGAACCUCCGUGGGA | 5482 | 5500 |
| UGNX1273 | 277 | CCACGGAGGUUCAGUUCCA | 278 | UGGAACUGAACCUCCGUGG | 5484 | 5502 |
| UGNX1274 | 279 | CACGGAGGUUCAGUUCCAC | 280 | GUGGAACUGAACCUCCGUG | 5485 | 5503 |
| UGNX1275 | 281 | ACGGAGGUUCAGUUCCACA | 282 | UGUGGAACUGAACCUCCGU | 5486 | 5504 |
| UGNX1276 | 283 | CGGAGGUUCAGUUCCACAC | 284 | GUGUGGAACUGAACCUCCG | 5487 | 5505 |
| UGNX1277 | 285 | GGAGGUUCAGUUCCACACU | 286 | AGUGUGGAACUGAACCUCC | 5488 | 5506 |
| UGNX1278 | 287 | GAGGUUCAGUUCCACACUC | 288 | GAGUGUGGAACUGAACCUC | 5489 | 5507 |
| UGNX1279 | 289 | AGGUUCAGUUCCACACUCC | 290 | GGAGUGUGGAACUGAACCU | 5490 | 5508 |
| UGNX1280 | 291 | GGUUCAGUUCCACACUCCC | 292 | GGGAGUGUGGAACUGAACC | 5491 | 5509 |
| UGNX1441 | 293 | UUCCUCUUCGUCUCUCCGG | 294 | CCGGAGAGACGAAGAGGAA | 5652 | 5670 |
| UGNX1598 | 295 | GCUCACCGCCAUUCAUGAA | 296 | UUCAUGAAUGGCGGUGAGC | 5809 | 5827 |
| UGNX1600 | 297 | UCACCGCCAUUCAUGAAGG | 298 | CCUUCAUGAAUGGCGGUGA | 5811 | 5829 |
| UGNX1601 | 299 | CACCGCCAUUCAUGAAGGG | 300 | CCCUUCAUGAAUGGCGGUG | 5812 | 5830 |
| UGNX1632 | 301 | UGCCUGUGGGCCUUUACAA | 302 | UUGUAAAGGCCCACAGGCA | 5843 | 5861 |
| UGNX1633 | 303 | GCCUGUGGGCCUUUACAAG | 304 | CUUGUAAAGGCCCACAGGC | 5844 | 5862 |
| UGNX1634 | 305 | CCUGUGGGCCUUUACAAGG | 306 | CCUUGUAAAGGCCCACAGG | 5845 | 5863 |
| UGNX1635 | 307 | CUGUGGGCCUUUACAAGGG | 308 | CCCUUGUAAAGGCCCACAG | 5846 | 5864 |
| UGNX1636 | 309 | UGUGGGCCUUUACAAGGGC | 310 | GCCCUUGUAAAGGCCCACA | 5847 | 5865 |
| UGNX1759 | 311 | CGUCCGUCCGUGAAAUUCC | 312 | GGAAUUUCACGGACGGACG | 5970 | 5988 |
| UGNX1760 | 313 | GUCCGUCCGUGAAAUUCCG | 314 | CGGAAUUUCACGGACGGAC | 5971 | 5989 |
| UGNX1761 | 315 | UCCGUCCGUGAAAUUCCGG | 316 | CCGGAAUUUCACGGACGGA | 5972 | 5990 |
| UGNX1856 | 317 | GACGGAGACUCGUUUGGAC | 318 | GUCCAAACGAGUCUCCGUC | 6067 | 6085 |
| UGNX1857 | 319 | ACGGAGACUCGUUUGGACC | 320 | GGUCCAAACGAGUCUCCGU | 6068 | 6086 |
| UGNX1899 | 321 | AGCCUGCUUUGAGCGGAAC | 322 | GUUCCGCUCAAAGCAGGCU | 6110 | 6128 |
| UGNX1904 | 323 | GCUUUGAGCGGAACCCGUA | 324 | UACGGGUUCCGCUCAAAGC | 6115 | 6133 |
| UGNX1905 | 325 | CUUUGAGCGGAACCCGUAC | 326 | GUACGGGUUCCGCUCAAAG | 6116 | 6134 |
| UGNX1906 | 327 | UUUGAGCGGAACCCGUACC | 328 | GGUACGGGUUCCGCUCAAA | 6117 | 6135 |
| UGNX1975 | 329 | CCCAGGGUCCAGAUUUGGU | 330 | ACCAAAUCUGGACCCUGGG | 6186 | 6204 |
| UGNX1976 | 331 | CCAGGGUCCAGAUUUGGUU | 332 | AACCAAAUCUGGACCCUGG | 6187 | 6205 |
| UGNX1977 | 333 | CAGGGUCCAGAUUUGGUUU | 334 | AAACCAAAUCUGGACCCUG | 6188 | 6206 |
| UGNX1978 | 335 | AGGGUCCAGAUUUGGUUUC | 336 | GAAACCAAAUCUGGACCCU | 6189 | 6207 |
| UGNX1979 | 337 | GGGUCCAGAUUUGGUUUCA | 338 | UGAAACCAAAUCUGGACCC | 6190 | 6208 |
| UGNX1980 | 339 | GGUCCAGAUUUGGUUUCAG | 340 | CUGAAACCAAAUCUGGACC | 6191 | 6209 |
| UGNX1981 | 341 | GUCCAGAUUUGGUUUCAGA | 342 | UCUGAAACCAAAUCUGGAC | 6192 | 6210 |
| UGNX1982 | 343 | UCCAGAUUUGGUUUCAGAA | 344 | UUCUGAAACCAAAUCUGGA | 6193 | 6211 |
| UGNX1983 | 345 | CCAGAUUUGGUUUCAGAAU | 346 | AUUCUGAAACCAAAUCUGG | 6194 | 6212 |
| UGNX1984 | 347 | CAGAUUUGGUUUCAGAAUG | 348 | CAUUCUGAAACCAAAUCUG | 6195 | 6213 |
| UGNX1985 | 349 | AGAUUUGGUUUCAGAAUGA | 350 | UCAUUCUGAAACCAAAUCU | 6196 | 6214 |
| UGNX1986 | 351 | GAUUUGGUUUCAGAAUGAG | 352 | CUCAUUCUGAAACCAAAUC | 6197 | 6215 |
| UGNX1987 | 353 | AUUUGGUUUCAGAAUGAGA | 354 | UCUCAUUCUGAAACCAAAU | 6198 | 6216 |
| UGNX1988 | 355 | UUUGGUUUCAGAAUGAGAG | 356 | CUCUCAUUCUGAAACCAAA | 6199 | 6217 |
| UGNX1989 | 357 | UUGGUUUCAGAAUGAGAGG | 358 | CCUCUCAUUCUGAAACCAA | 6200 | 6218 |
| UGNX1990 | 359 | UGGUUUCAGAAUGAGAGGU | 360 | ACCUCUCAUUCUGAAACCA | 6201 | 6219 |
| UGNX1991 | 361 | GGUUUCAGAAUGAGAGGUC | 362 | GACCUCUCAUUCUGAAACC | 6202 | 6220 |
| UGNX1992 | 363 | GUUUCAGAAUGAGAGGUCA | 364 | UGACCUCUCAUUCUGAAAC | 6203 | 6221 |
| UGNX1993 | 365 | UUUCAGAAUGAGAGGUCAC | 366 | GUGACCUCUCAUUCUGAAA | 6204 | 6222 |
| UGNX1994 | 367 | UUCAGAAUGAGAGGUCACG | 368 | CGUGACCUCUCAUUCUGAA | 6205 | 6223 |
| UGNX1995 | 369 | UCAGAAUGAGAGGUCACGC | 370 | GCGUGACCUCUCAUUCUGA | 6206 | 6224 |
| UGNX1996 | 371 | CAGAAUGAGAGGUCACGCC | 372 | GGCGUGACCUCUCAUUCUG | 6207 | 6225 |
| UGNX1997 | 373 | AGAAUGAGAGGUCACGCCA | 374 | UGGCGUGACCUCUCAUUCU | 6208 | 6226 |
| UGNX1998 | 375 | GAAUGAGAGGUCACGCCAG | 376 | CUGGCGUGACCUCUCAUUC | 6209 | 6227 |
| UGNX1999 | 377 | AAUGAGAGGUCACGCCAGC | 378 | GCUGGCGUGACCUCUCAUU | 6210 | 6228 |
| UGNX2000 | 379 | AUGAGAGGUCACGCCAGCU | 380 | AGCUGGCGUGACCUCUCAU | 6211 | 6229 |
| UGNX2117 | 381 | UGCUCCUCCGAGCCUUUGA | 382 | UCAAAGGCUCGGAGGAGCA | 6328 | 6346 |
| UGNX2120 | 383 | UCCUCCGAGCCUUUGAGAA | 384 | UUCUCAAAGGCUCGGAGGA | 6331 | 6349 |
| UGNX2121 | 385 | CCUCCGAGCCUUUGAGAAG | 386 | CUUCUCAAAGGCUCGGAGG | 6332 | 6350 |
| UGNX2122 | 387 | CUCCGAGCCUUUGAGAAGG | 388 | CCUUCUCAAAGGCUCGGAG | 6333 | 6351 |
| UGNX2123 | 389 | UCCGAGCCUUUGAGAAGGA | 390 | UCCUUCUCAAAGGCUCGGA | 6334 | 6352 |
| UGNX2124 | 391 | CCGAGCCUUUGAGAAGGAU | 392 | AUCCUUCUCAAAGGCUCGG | 6335 | 6353 |
| UGNX2125 | 393 | CGAGCCUUUGAGAAGGAUC | 394 | GAUCCUUCUCAAAGGCUCG | 6336 | 6354 |
| UGNX2126 | 395 | GAGCCUUUGAGAAGGAUCG | 396 | CGAUCCUUCUCAAAGGCUC | 6337 | 6355 |
| UGNX2127 | 397 | AGCCUUUGAGAAGGAUCGC | 398 | GCGAUCCUUCUCAAAGGCU | 6338 | 6356 |
| UGNX2128 | 399 | GCCUUUGAGAAGGAUCGCU | 400 | AGCGAUCCUUCUCAAAGGC | 6339 | 6357 |
| UGNX2129 | 401 | CCUUUGAGAAGGAUCGCUU | 402 | AAGCGAUCCUUCUCAAAGG | 6340 | 6358 |
| UGNX2130 | 403 | CUUUGAGAAGGAUCGCUUU | 404 | AAAGCGAUCCUUCUCAAAG | 6341 | 6359 |
| UGNX2131 | 405 | UUUGAGAAGGAUCGCUUUC | 406 | GAAAGCGAUCCUUCUCAAA | 6342 | 6360 |
| UGNX2132 | 407 | UUGAGAAGGAUCGCUUUCC | 408 | GGAAAGCGAUCCUUCUCAA | 6343 | 6361 |
| UGNX2133 | 409 | UGAGAAGGAUCGCUUUCCA | 410 | UGGAAAGCGAUCCUUCUCA | 6344 | 6362 |
| UGNX2134 | 411 | GAGAAGGAUCGCUUUCCAG | 412 | CUGGAAAGCGAUCCUUCUC | 6345 | 6363 |
| UGNX2135 | 413 | AGAAGGAUCGCUUUCCAGG | 414 | CCUGGAAAGCGAUCCUUCU | 6346 | 6364 |
| UGNX2136 | 415 | GAAGGAUCGCUUUCCAGGC | 416 | GCCUGGAAAGCGAUCCUUC | 6347 | 6365 |
| UGNX2137 | 417 | AAGGAUCGCUUUCCAGGCA | 418 | UGCCUGGAAAGCGAUCCUU | 6348 | 6366 |
| UGNX2138 | 419 | AGGAUCGCUUUCCAGGCAU | 420 | AUGCCUGGAAAGCGAUCCU | 6349 | 6367 |
| UGNX2139 | 421 | GGAUCGCUUUCCAGGCAUC | 422 | GAUGCCUGGAAAGCGAUCC | 6350 | 6368 |
| UGNX2140 | 423 | GAUCGCUUUCCAGGCAUCG | 424 | CGAUGCCUGGAAAGCGAUC | 6351 | 6369 |
| UGNX2194 | 425 | CCGGAGUCCAGGAUUCAGA | 426 | UCUGAAUCCUGGACUCCGG | 6405 | 6423 |
| UGNX2195 | 427 | CGGAGUCCAGGAUUCAGAU | 428 | AUCUGAAUCCUGGACUCCG | 6406 | 6424 |
| UGNX2196 | 429 | GGAGUCCAGGAUUCAGAUC | 430 | GAUCUGAAUCCUGGACUCC | 6407 | 6425 |
| UGNX2197 | 431 | GAGUCCAGGAUUCAGAUCU | 432 | AGAUCUGAAUCCUGGACUC | 6408 | 6426 |
| UGNX2198 | 433 | AGUCCAGGAUUCAGAUCUG | 434 | CAGAUCUGAAUCCUGGACU | 6409 | 6427 |
| UGNX2199 | 435 | GUCCAGGAUUCAGAUCUGG | 436 | CCAGAUCUGAAUCCUGGAC | 6410 | 6428 |
| UGNX2200 | 437 | UCCAGGAUUCAGAUCUGGU | 438 | ACCAGAUCUGAAUCCUGGA | 6411 | 6429 |
| UGNX2201 | 439 | CCAGGAUUCAGAUCUGGUU | 440 | AACCAGAUCUGAAUCCUGG | 6412 | 6430 |
| UGNX2202 | 441 | CAGGAUUCAGAUCUGGUUU | 442 | AAACCAGAUCUGAAUCCUG | 6413 | 6431 |
| UGNX2203 | 443 | AGGAUUCAGAUCUGGUUUC | 444 | GAAACCAGAUCUGAAUCCU | 6414 | 6432 |
| UGNX2204 | 445 | GGAUUCAGAUCUGGUUUCA | 446 | UGAAACCAGAUCUGAAUCC | 6415 | 6433 |
| UGNX2205 | 447 | GAUUCAGAUCUGGUUUCAG | 448 | CUGAAACCAGAUCUGAAUC | 6416 | 6434 |
| UGNX2206 | 449 | AUUCAGAUCUGGUUUCAGA | 450 | UCUGAAACCAGAUCUGAAU | 6417 | 6435 |
| UGNX2207 | 451 | UUCAGAUCUGGUUUCAGAA | 452 | UUCUGAAACCAGAUCUGAA | 6418 | 6436 |
| UGNX2208 | 453 | UCAGAUCUGGUUUCAGAAU | 454 | AUUCUGAAACCAGAUCUGA | 6419 | 6437 |
| UGNX2209 | 455 | CAGAUCUGGUUUCAGAAUC | 456 | GAUUCUGAAACCAGAUCUG | 6420 | 6438 |
| UGNX2210 | 457 | AGAUCUGGUUUCAGAAUCG | 458 | CGAUUCUGAAACCAGAUCU | 6421 | 6439 |
| UGNX2211 | 459 | GAUCUGGUUUCAGAAUCGA | 460 | UCGAUUCUGAAACCAGAUC | 6422 | 6440 |
| UGNX2212 | 461 | AUCUGGUUUCAGAAUCGAA | 462 | UUCGAUUCUGAAACCAGAU | 6423 | 6441 |
| UGNX2213 | 463 | UCUGGUUUCAGAAUCGAAG | 464 | CUUCGAUUCUGAAACCAGA | 6424 | 6442 |
| UGNX2214 | 465 | CUGGUUUCAGAAUCGAAGG | 466 | CCUUCGAUUCUGAAACCAG | 6425 | 6443 |
| UGNX2215 | 467 | UGGUUUCAGAAUCGAAGGG | 468 | CCCUUCGAUUCUGAAACCA | 6426 | 6444 |
| UGNX2216 | 469 | GGUUUCAGAAUCGAAGGGC | 470 | GCCCUUCGAUUCUGAAACC | 6427 | 6445 |
| UGNX2217 | 471 | GUUUCAGAAUCGAAGGGCC | 472 | GGCCCUUCGAUUCUGAAAC | 6428 | 6446 |
| UGNX2218 | 473 | UUUCAGAAUCGAAGGGCCA | 474 | UGGCCCUUCGAUUCUGAAA | 6429 | 6447 |
| UGNX2219 | 475 | UUCAGAAUCGAAGGGCCAG | 476 | CUGGCCCUUCGAUUCUGAA | 6430 | 6448 |
| UGNX2220 | 477 | UCAGAAUCGAAGGGCCAGG | 478 | CCUGGCCCUUCGAUUCUGA | 6431 | 6449 |
| UGNX2222 | 479 | AGAAUCGAAGGGCCAGGCA | 480 | UGCCUGGCCCUUCGAUUCU | 6433 | 6451 |
| UGNX2951 | 481 | AGGCGCAACCUCUCCUAGA | 482 | UCUAGGAGAGGUUGCGCCU | 7162 | 7180 |
| UGNX2953 | 483 | GCGCAACCUCUCCUAGAAA | 484 | UUUCUAGGAGAGGUUGCGC | 7164 | 7182 |
| UGNX2954 | 485 | CGCAACCUCUCCUAGAAAC | 486 | GUUUCUAGGAGAGGUUGCG | 7165 | 7183 |
| UGNX2955 | 487 | GCAACCUCUCCUAGAAACG | 488 | CGUUUCUAGGAGAGGUUGC | 7166 | 7184 |
| UGNX2956 | 489 | CAACCUCUCCUAGAAACGG | 490 | CCGUUUCUAGGAGAGGUUG | 7167 | 7185 |
| UGNX2957 | 491 | AACCUCUCCUAGAAACGGA | 492 | UCCGUUUCUAGGAGAGGUU | 7168 | 7186 |
| UGNX2958 | 493 | ACCUCUCCUAGAAACGGAG | 494 | CUCCGUUUCUAGGAGAGGU | 7169 | 7187 |
| UGNX2959 | 495 | CCUCUCCUAGAAACGGAGG | 496 | CCUCCGUUUCUAGGAGAGG | 7170 | 7188 |
| UGNX2960 | 497 | CUCUCCUAGAAACGGAGGC | 498 | GCCUCCGUUUCUAGGAGAG | 7171 | 7189 |
| UGNX2961 | 499 | UCUCCUAGAAACGGAGGCC | 500 | GGCCUCCGUUUCUAGGAGA | 7172 | 7190 |
| UGNX3029 | 501 | UCAGCGAGGAAGAAUACCG | 502 | CGGUAUUCUUCCUCGCUGA | 7240 | 7258 |
| UGNX3030 | 503 | CAGCGAGGAAGAAUACCGG | 504 | CCGGUAUUCUUCCUCGCUG | 7241 | 7259 |
| UGNX3031 | 505 | AGCGAGGAAGAAUACCGGG | 506 | CCCGGUAUUCUUCCUCGCU | 7242 | 7260 |
| UGNX3033 | 507 | CGAGGAAGAAUACCGGGCU | 508 | AGCCCGGUAUUCUUCCUCG | 7244 | 7262 |
| UGNX3034 | 509 | GAGGAAGAAUACCGGGCUC | 510 | GAGCCCGGUAUUCUUCCUC | 7245 | 7263 |
| UGNX3035 | 511 | AGGAAGAAUACCGGGCUCU | 512 | AGAGCCCGGUAUUCUUCCU | 7246 | 7264 |
| UGNX3036 | 513 | GGAAGAAUACCGGGCUCUG | 514 | CAGAGCCCGGUAUUCUUCC | 7247 | 7265 |
| UGNX3037 | 515 | GAAGAAUACCGGGCUCUGC | 516 | GCAGAGCCCGGUAUUCUUC | 7248 | 7266 |
| UGNX3038 | 517 | AAGAAUACCGGGCUCUGCU | 518 | AGCAGAGCCCGGUAUUCUU | 7249 | 7267 |
| UGNX3039 | 519 | AGAAUACCGGGCUCUGCUG | 520 | CAGCAGAGCCCGGUAUUCU | 7250 | 7268 |
| UGNX3041 | 521 | AAUACCGGGCUCUGCUGGA | 522 | UCCAGCAGAGCCCGGUAUU | 7252 | 7270 |
| UGNX3049 | 523 | GCUCUGCUGGAGGAGCUUU | 524 | AAAGCUCCUCCAGCAGAGC | 7260 | 7278 |
| UGNX3050 | 525 | CUCUGCUGGAGGAGCUUUA | 526 | UAAAGCUCCUCCAGCAGAG | 7261 | 7279 |
| UGNX3051 | 527 | UCUGCUGGAGGAGCUUUAG | 528 | CUAAAGCUCCUCCAGCAGA | 7262 | 7280 |
| UGNX3052 | 529 | CUGCUGGAGGAGCUUUAGG | 530 | CCUAAAGCUCCUCCAGCAG | 7263 | 7281 |
| UGNX3053 | 531 | UGCUGGAGGAGCUUUAGGA | 532 | UCCUAAAGCUCCUCCAGCA | 7264 | 7282 |
| UGNX3054 | 533 | GCUGGAGGAGCUUUAGGAC | 534 | GUCCUAAAGCUCCUCCAGC | 7265 | 7283 |
| UGNX3055 | 535 | CUGGAGGAGCUUUAGGACG | 536 | CGUCCUAAAGCUCCUCCAG | 7266 | 7284 |
| UGNX3056 | 537 | UGGAGGAGCUUUAGGACGC | 538 | GCGUCCUAAAGCUCCUCCA | 7267 | 7285 |
| UGNX3193 | 539 | CCUGGGAUUCCUGCCUUCU | 540 | AGAAGGCAGGAAUCCCAGG | 7404 | 7422 |
| UGNX3195 | 541 | UGGGAUUCCUGCCUUCUAG | 542 | CUAGAAGGCAGGAAUCCCA | 7406 | 7424 |
| UGNX3196 | 543 | GGGAUUCCUGCCUUCUAGG | 544 | CCUAGAAGGCAGGAAUCCC | 7407 | 7425 |
| UGNX3197 | 545 | GGAUUCCUGCCUUCUAGGU | 546 | ACCUAGAAGGCAGGAAUCC | 7408 | 7426 |
| UGNX3198 | 547 | GAUUCCUGCCUUCUAGGUC | 548 | GACCUAGAAGGCAGGAAUC | 7409 | 7427 |
| UGNX3199 | 549 | AUUCCUGCCUUCUAGGUCU | 550 | AGACCUAGAAGGCAGGAAU | 7410 | 7428 |
| UGNX3200 | 551 | UUCCUGCCUUCUAGGUCUA | 552 | UAGACCUAGAAGGCAGGAA | 7411 | 7429 |
| UGNX3201 | 553 | UCCUGCCUUCUAGGUCUAG | 554 | CUAGACCUAGAAGGCAGGA | 7412 | 7430 |
| UGNX3202 | 555 | CCUGCCUUCUAGGUCUAGG | 556 | CCUAGACCUAGAAGGCAGG | 7413 | 7431 |
| UGNX3203 | 557 | CUGCCUUCUAGGUCUAGGC | 558 | GCCUAGACCUAGAAGGCAG | 7414 | 7432 |
| UGNX3204 | 559 | UGCCUUCUAGGUCUAGGCC | 560 | GGCCUAGACCUAGAAGGCA | 7415 | 7433 |
| UGNX3208 | 561 | UUCUAGGUCUAGGCCCGGU | 562 | ACCGGGCCUAGACCUAGAA | 7419 | 7437 |
| UGNX3241 | 563 | CGCGGAGAACUGCCAUUCU | 564 | AGAAUGGCAGUUCUCCGCG | 7452 | 7470 |
| UGNX3243 | 565 | CGGAGAACUGCCAUUCUUU | 566 | AAAGAAUGGCAGUUCUCCG | 7454 | 7472 |
| UGNX3244 | 567 | GGAGAACUGCCAUUCUUUC | 568 | GAAAGAAUGGCAGUUCUCC | 7455 | 7473 |
| UGNX3245 | 569 | GAGAACUGCCAUUCUUUCC | 570 | GGAAAGAAUGGCAGUUCUC | 7456 | 7474 |
| UGNX3246 | 571 | AGAACUGCCAUUCUUUCCU | 572 | AGGAAAGAAUGGCAGUUCU | 7457 | 7475 |
| UGNX3247 | 573 | GAACUGCCAUUCUUUCCUG | 574 | CAGGAAAGAAUGGCAGUUC | 7458 | 7476 |
| UGNX3248 | 575 | AACUGCCAUUCUUUCCUGG | 576 | CCAGGAAAGAAUGGCAGUU | 7459 | 7477 |
| UGNX3249 | 577 | ACUGCCAUUCUUUCCUGGG | 578 | CCCAGGAAAGAAUGGCAGU | 7460 | 7478 |
| UGNX3250 | 579 | CUGCCAUUCUUUCCUGGGC | 580 | GCCCAGGAAAGAAUGGCAG | 7461 | 7479 |
| UGNX3251 | 581 | UGCCAUUCUUUCCUGGGCA | 582 | UGCCCAGGAAAGAAUGGCA | 7462 | 7480 |
| UGNX3252 | 583 | GCCAUUCUUUCCUGGGCAU | 584 | AUGCCCAGGAAAGAAUGGC | 7463 | 7481 |
| UGNX3253 | 585 | CCAUUCUUUCCUGGGCAUC | 586 | GAUGCCCAGGAAAGAAUGG | 7464 | 7482 |
| UGNX3254 | 587 | CAUUCUUUCCUGGGCAUCC | 588 | GGAUGCCCAGGAAAGAAUG | 7465 | 7483 |
| UGNX3255 | 589 | AUUCUUUCCUGGGCAUCCC | 590 | GGGAUGCCCAGGAAAGAAU | 7466 | 7484 |
| UGNX3256 | 591 | UUCUUUCCUGGGCAUCCCG | 592 | CGGGAUGCCCAGGAAAGAA | 7467 | 7485 |

### Example 2: Silencing of DUX4 Gene by Double Stranded siRNA

The double-stranded small interfering RNA (siRNA) listed in Table 1 were tested for inhibition of DUX4 gene expression in vitro as described below.

Cells and Cell Culture. Immortalized human FSHD1 (54-2) (Krom et aL, 2012) and FSHD2 (MB200) (Stadler et al., 2011) cell lines were used. Immortalized myoblasts were grown in Ham's F-10 Nutrient Mix (Gibco, Waltham, MA, USA) supplemented with 20% Corning USDA Approved Source Fetal Bovine Serum (Corning, Corning, NY, USA), 100 U/100 g penicillin/streptomycin (Gibco), 10 ng/ml recombinant human fibroblast growth factor (Promega Corporation, Madison, WI, USA) and 1 μM dexamethasone (Sigma-Aldrich). Differentiation of myoblasts into myotubes was achieved by switching confluent myoblast monolayers into DMEM:F-12 Nutrient Mixture (1:1, Gibco) supplemented with 2% KnockOut Serum Replacement (Gibco), 100 U/100 μg penicillin/streptomycin, 10 μg/ml insulin and 10 μg/ml transferrin (KSR media) for 40 hours.

Small interfering RNA (siRNA) transfections. Unmodified duplex 21-mer siRNAs listed in Table 1 containing 19 base-pair complementary sequences and dTdT 3 prime overhangs were synthesized and obtained from Thermo Fisher Scientific or Integrated DNA Technologies. Transfections of siRNAs into FSHD1 and FSHD2 myoblasts were carried out using Lipofectamine RNAiMAX (Invitrogen) according to the manufacturer's instructions. Briefly, cells were seeded at 1×10⁵ cells/well in 12-well plates and transfected approximately 20 hours later with 2 μl Lipofectamine RNAiMAX and 10 pmol or less of either gene-specific siRNAs or a scrambled non-silencing control siRNA diluted in 100 μl Opti-MEM Reduced Serum Medium. 24, 48 or 72 hours following transfection, cells were incubated in differentiation medium for 40 hours to induce differentiation into myotubes and harvested for total RNA analysis.

Measurement of DUX4 target gene expression in FSHD1 and FSHD2 myotubes. Total RNA was extracted from whole cells using the RNeasy Mini Kit (Qiagen, Hilden, Germany) according to the manufacturer's instructions. Isolated RNA was treated with DNase I (Thermo Fisher Scientific), heat inactivated and reverse transcribed into cDNA using Superscript III (Thermo Fisher Scientific) and oligo(dT) primers (Invitrogen) following the manufacturer's protocol. qPCR was performed on cDNA to measure expression of DUX4. Expression of DUX4 can be measured by measuring expression of genes that are upregulated by DUX4, e.g., MBD3L2, ZSCAN4, LEUTX, MYOG, and MYH2 using TaqMan Gene Expression Assay ID numbers: MBD3L2, Hs00544743_m1, MYH2, Hs00430042_m1 MYOG, Hs01072232_m1; RPL30, Hs00265497_m1; LEUTX, Hs01028718_m1; ZSCAN4, Hs00537549_m1 or DUX4 with primers GCCGGCCCAGGTACCA (SEQ ID NO: 594) and CAGCGAGCTCCCTTGCA (SEQ ID NO: 595) with probe CAGTGCGCACCCCG (SEQ ID NO: 596) having a florescent dye (6FAM) attached at the 5′ end of the probe and a minor groove binder (MGB) and a non-florescent quencher (NFQ) attached at the 3′ end of the probe.

The results are summarized in Tables 2 and 3 below. MB200 or 54-2 myoblasts were transfected as described above with an siRNA listed in Table 1. RNA was isolated and analyzed by qRT-PCR. The results for MBD3L2 expression are shown in Tables 2 and 3 below as percent expression relative to the expression in cells that were transfected with the control. The results show that some antisense siRNAs inhibited DUX4 expression.

TABLE 2
% Inhibition in MB200 Cells

| | SEQ | sense | SEQ | antisense | % |
|---|---|---|---|---|---|
| | ID | | ID | | inhibi- |
| Oligo name | NO. | | NO: | | tion |
| UGNX394 | 1 | GGCAGAGAUGGAGAGAGGA | 2 | UCCUCUCUCCAUCUCUGCC | 36 |
| UGNX482 | 3 | AGGGAGGAACGGAGGGAAA | 4 | UUUCCCUCCGUUCCUCCCU | 41 |
| UGNX554 | 5 | GAGGAAGGCAGGGAGGAAA | 6 | UUUCCUCCCUGCCUUCCUC | 39 |
| UGNX722 | 7 | CGGUUUCCUCCGGGACAAA | 8 | UUUGUCCCGGAGGAAACCG | 49 |
| UGNX779 | 9 | CGGUUCACAGACCGCACAU | 10 | AUGUGCGGUCUGUGAACCG | −4 |
| UGNX864 | 11 | GACGACGGAGGCGUGAUUU | 12 | AAAUCACGCCUCCGUCGUC | 45 |
| UGNX916 | 13 | GCCUGUUGCUCACGUCUCU | 14 | AGAGACGUGAGCAACAGGC | 50 |
| UGNX950 | 15 | CUGGCCAUGCCGACUGUUU | 16 | AAACAGUCGGCAUGGCCAG | 50 |
| UGNX991 | 17 | CCGGAAACAUGCAGGGAAG | 18 | CUUCCCUGCAUGUUUCCGG | 67 |
| UGNX1032 | 19 | UUCGCUCUCCUUGCCAGGU | 20 | ACCUGGCAAGGAGAGCGAA | 44 |
| UGNX1094 | 21 | GGAAUCCAUCGUCAGGCCA | 22 | UGGCCUGACGAUGGAUUCC | 55 |
| UGNX1142 | 23 | GUCUCGCUCUGGUCUUCUA | 24 | UAGAAGACCAGAGCGAGAC | 55 |
| UGNX1223 | 25 | CACAGGCAUUGCCUCCUUC | 26 | GAAGGAGGCAAUGCCUGUG | 27 |
| UGNX1253 | 27 | GCCUGGCACACUCAAGACU | 28 | AGUCUUGAGUGUGCCAGGC | 58 |
| UGNX1311 | 29 | AGGCUGGUUUCUCCCUGCU | 30 | AGCAGGGAGAAACCAGCCU | 66 |
| UGNX1440 | 31 | CUUCCUCUUCGUCUCUCCG | 32 | CGGAGAGACGAAGAGGAAG | 51 |
| UGNX1599 | 33 | CUCACCGCCAUUCAUGAAG | 34 | CUUCAUGAAUGGCGGUGAG | 68 |
| UGNX1631 | 35 | CUGCCUGUGGGCCUUUACA | 36 | UGUAAAGGCCCACAGGCAG | 75 |
| UGNX1758 | 37 | GCGUCCGUCCGUGAAAUUC | 38 | GAAUUUCACGGACGGACGC | 92 |
| UGNX1855 | 39 | CGACGGAGACUCGUUUGGA | 40 | UCCAAACGAGUCUCCGUCG | 85 |
| UGNX1898 | 41 | GAGCCUGCUUUGAGCGGAA | 42 | UUCCGCUCAAAGCAGGCUC | 86 |
| UGNX1972 | 43 | GAGCCCAGGGUCCAGAUUU | 44 | AAAUCUGGACCCUGGGCUC | 56 |
| UGNX2119 | 45 | CUCCUCCGAGCCUUUGAGA | 46 | UCUCAAAGGCUCGGAGGAG | 61 |
| UGNX2192 | 47 | UCCCGGAGUCCAGGAUUCA | 48 | UGAAUCCUGGACUCCGGGA | 66 |
| UGNX2909 | 49 | UGCUGCUGGAUGAGCUCCU | 50 | GGAGCUCAUCCAGCAGCA | 61 |
| UGNX2952 | 51 | GGCGCAACCUCUCCUAGAA | 52 | UUCUAGGAGAGGUUGCGCC | 89 |
| UGNX3028 | 53 | CUCAGCGAGGAAGAAUACC | 54 | GGUAUUCUUCCUCGCUGAG | 35 |
| UGNX3194 | 55 | CUGGGAUUCCUGCCUUCUA | 56 | UAGAAGGCAGGAAUCCCAG | 69 |
| UGNX3242 | 57 | GCGGAGAACUGCCAUUCUU | 58 | AAGAAUGGCAGUUCUCCGC | 84 |
| UGNX395 | 59 | GCAGAGAUGGAGAGAGGAA | 60 | UUCCUCUCUCCAUCUCUGC | 40 |
| UGNX396 | 61 | CAGAGAUGGAGAGAGGAAC | 62 | GUUCCUCUCUCCAUCUCUG | 74 |
| UGNX397 | 63 | AGAGAUGGAGAGAGGAACG | 64 | CGUUCCUCUCUCCAUCUCU | 21 |
| UGNX398 | 65 | GAGAUGGAGAGAGGAACGG | 66 | CCGUUCCUCUCUCCAUCUC | 80 |
| UGNX399 | 67 | AGAUGGAGAGAGGAACGGG | 68 | CCCGUUCCUCUCUCCAUCU | 65 |
| UGNX400 | 69 | GAUGGAGAGAGGAACGGGA | 70 | UCCCGUUCCUCUCUCCAUC | 73 |
| UGNX401 | 71 | AUGGAGAGAGGAACGGGAG | 72 | CUCCCGUUCCUCUCUCCAU | 81 |
| UGNX402 | 73 | UGGAGAGAGGAACGGGAGA | 74 | UCUCCCGUUCCUCUCUCCA | 91 |
| UGNX405 | 75 | AGAGAGGAACGGGAGACCU | 76 | AGGUCUCCCGUUCCUCUCU | 77 |
| UGNX406 | 77 | GAGAGGAACGGGAGACCUA | 78 | UAGGUCUCCCGUUCCUCUC | 90 |
| UGNX407 | 79 | AGAGGAACGGGAGACCUAG | 80 | CUAGGUCUCCCGUUCCUCU | 74 |
| UGNX408 | 81 | GAGGAACGGGAGACCUAGA | 82 | UCUAGGUCUCCCGUUCCUC | 56 |
| UGNX409 | 83 | AGGAACGGGAGACCUAGAG | 84 | CUCUAGGUCUCCCGUUCCU | 57 |
| UGNX484 | 85 | GGAGGAACGGAGGGAAAGA | 86 | UCUUUCCCUCCGUUCCUCC | 64 |
| UGNX485 | 87 | GAGGAACGGAGGGAAAGAC | 88 | GUCUUUCCCUCCGUUCCUC | 72 |
| UGNX486 | 89 | AGGAACGGAGGGAAAGACA | 90 | UGUCUUUCCCUCCGUUCCU | 42 |
| UGNX487 | 91 | GGAACGGAGGGAAAGACAG | 92 | CUGUCUUUCCCUCCGUUCC | 80 |
| UGNX488 | 93 | GAACGGAGGGAAAGACAGA | 94 | UCUGUCUUUCCCUCCGUUC | 96 |
| UGNX489 | 95 | AACGGAGGGAAAGACAGAG | 96 | CUCUGUCUUUCCCUCCGUU | 79 |
| UGNX490 | 97 | ACGGAGGGAAAGACAGAGC | 98 | GCUCUGUCUUUCCCUCCGU | 81 |
| UGNX492 | 99 | GGAGGGAAAGACAGAGCGA | 100 | UCGCUCUGUCUUUCCCUCC | 25 |
| UGNX493 | 101 | GAGGGAAAGACAGAGCGAC | 102 | GUCGCUCUGUCUUUCCCUC | 80 |
| UGNX494 | 103 | AGGGAAAGACAGAGCGACG | 104 | CGUCGCUCUGUCUUUCCCU | 38 |
| UGNX496 | 105 | GGAAAGACAGAGCGACGCA | 106 | UGCGUCGCUCUGUCUUUCC | 47 |
| UGNX497 | 107 | GAAAGACAGAGCGACGCAG | 108 | CUGCGUCGCUCUGUCUUUC | 41 |
| UGNX498 | 109 | AAAGACAGAGCGACGCAGG | 110 | CCUGCGUCGCUCUGUCUUU | 63 |
| UGNX780 | 111 | GGUUCACAGACCGCACAUC | 112 | GAUGUGCGGUCUGUGAACC | 47 |
| UGNX781 | 113 | GUUCACAGACCGCACAUCC | 114 | GGAUGUGCGGUCUGUGAAC | 93 |
| UGNX782 | 115 | UUCACAGACCGCACAUCCC | 116 | GGGAUGUGCGGUCUGUGAA | 97 |
| UGNX917 | 117 | CCUGUUGCUCACGUCUCUC | 118 | GAGAGACGUGAGCAACAGG | −5 |
| UGNX918 | 119 | CUGUUGCUCACGUCUCUCC | 120 | GGAGAGACGUGAGCAACAG | 31 |
| UGNX919 | 121 | UGUUGCUCACGUCUCUCCG | 122 | CGGAGAGACGUGAGCAACA | 18 |
| UGNX954 | 123 | CCAUGCCGACUGUUUGCUC | 124 | GAGCAAACAGUCGGCAUGG | 80 |
| UGNX955 | 125 | CAUGCCGACUGUUUGCUCC | 126 | GGAGCAAACAGUCGGCAUG | 48 |
| UGNX956 | 127 | AUGCCGACUGUUUGCUCCC | 128 | GGGAGCAAACAGUCGGCAU | 65 |
| UGNX964 | 129 | UGUUUGCUCCCGGAGCUCU | 130 | AGAGCUCCGGGAGCAAACA | 94 |
| UGNX990 | 131 | CCCGGAAACAUGCAGGGAA | 132 | UUCCCUGCAUGUUUCCGGG | 93 |
| UGNX992 | 133 | CGGAAACAUGCAGGGAAGG | 134 | CCUUCCCUGCAUGUUUCCG | 83 |
| UGNX993 | 135 | GGAAACAUGCAGGGAAGGG | 136 | CCCUUCCCUGCAUGUUUCC | 76 |
| UGNX994 | 137 | GAAACAUGCAGGGAAGGGU | 138 | ACCCUUCCCUGCAUGUUUC | 21 |
| UGNX995 | 139 | AAACAUGCAGGGAAGGGUG | 140 | CACCCUUCCCUGCAUGUUU | 43 |
| UGNX996 | 141 | AACAUGCAGGGAAGGGUGC | 142 | GCACCCUUCCCUGCAUGUU | 5 |
| UGNX998 | 143 | CAUGCAGGGAAGGGUGCAA | 144 | UUGCACCCUUCCCUGCAUG | 83 |
| UGNX999 | 145 | AUGCAGGGAAGGGUGCAAG | 146 | CUUGCACCCUUCCCUGCAU | 13 |
| UGNX1033 | 147 | UCGCUCUCCUUGCCAGGUU | 148 | AACCUGGCAAGGAGAGCGA | 57 |
| UGNX1036 | 149 | CUCUCCUUGCCAGGUUCCA | 150 | UGGAACCUGGCAAGGAGAG | 77 |
| UGNX1037 | 151 | UCUCCUUGCCAGGUUCCAA | 152 | UUGGAACCUGGCAAGGAGA | 42 |
| UGNX1038 | 153 | CUCCUUGCCAGGUUCCAAA | 154 | UUUGGAACCUGGCAAGGAG | 79 |
| UGNX1039 | 155 | UCCUUGCCAGGUUCCAAAC | 156 | GUUUGGAACCUGGCAAGGA | 51 |
| UGNX1040 | 157 | CCUUGCCAGGUUCCAAACC | 158 | GGUUUGGAACCUGGCAAGG | 36 |
| UGNX1041 | 159 | CUUGCCAGGUUCCAAACCG | 160 | CGGUUUGGAACCUGGCAAG | 42 |
| UGNX1042 | 161 | UUGCCAGGUUCCAAACCGG | 162 | CCGGUUUGGAACCUGGCAA | 89 |
| UGNX1049 | 163 | GUUCCAAACCGGCCACACU | 164 | AGUGUGGCCGGUUUGGAAC | 77 |
| UGNX1050 | 165 | UUCCAAACCGGCCACACUG | 166 | CAGUGUGGCCGGUUUGGAA | 54 |
| UGNX1095 | 167 | GAAUCCAUCGUCAGGCCAU | 168 | AUGGCCUGACGAUGGAUUC | 33 |
| UGNX1096 | 169 | AAUCCAUCGUCAGGCCAUC | 170 | GAUGGCCUGACGAUGGAUU | 89 |
| UGNX1097 | 171 | AUCCAUCGUCAGGCCAUCA | 172 | UGAUGGCCUGACGAUGGAU | 97 |
| UGNX1098 | 173 | UCCAUCGUCAGGCCAUCAC | 174 | GUGAUGGCCUGACGAUGGA | 55 |
| UGNX1143 | 175 | UCUCGCUCUGGUCUUCUAC | 176 | GUAGAAGACCAGAGCGAGA | 18 |
| UGNX1144 | 177 | CUCGCUCUGGUCUUCUACG | 178 | CGUAGAAGACCAGAGCGAG | 14 |
| UGNX1145 | 179 | UCGCUCUGGUCUUCUACGU | 180 | ACGUAGAAGACCAGAGCGA | 73 |
| UGNX1146 | 181 | CGCUCUGGUCUUCUACGUG | 182 | CACGUAGAAGACCAGAGCG | 93 |
| UGNX1147 | 183 | GCUCUGGUCUUCUACGUGG | 184 | CCACGUAGAAGACCAGAGC | −36 |
| UGNX1148 | 185 | CUCUGGUCUUCUACGUGGA | 186 | UCCACGUAGAAGACCAGAG | 65 |
| UGNX1149 | 187 | UCUGGUCUUCUACGUGGAA | 188 | UUCCACGUAGAAGACCAGA | 92 |
| UGNX1150 | 189 | CUGGUCUUCUACGUGGAAA | 190 | UUUCCACGUAGAAGACCAG | 37 |
| UGNX1151 | 191 | UGGUCUUCUACGUGGAAAU | 192 | AUUUCCACGUAGAAGACCA | 33 |
| UGNX1152 | 193 | GGUCUUCUACGUGGAAAUG | 194 | CAUUUCCACGUAGAAGACC | 66 |
| UGNX1153 | 195 | GUCUUCUACGUGGAAAUGA | 196 | UCAUUUCCACGUAGAAGAC | 34 |
| UGNX1154 | 197 | UCUUCUACGUGGAAAUGAA | 198 | UUCAUUUCCACGUAGAAGA | 80 |
| UGNX1155 | 199 | CUUCUACGUGGAAAUGAAC | 200 | GUUCAUUUCCACGUAGAAG | 65 |
| UGNX1156 | 201 | UUCUACGUGGAAAUGAACG | 202 | CGUUCAUUUCCACGUAGAA | 9 |
| UGNX1157 | 203 | UCUACGUGGAAAUGAACGA | 204 | UCGUUCAUUUCCACGUAGA | 66 |
| UGNX1158 | 205 | CUACGUGGAAAUGAACGAG | 206 | CUCGUUCAUUUCCACGUAG | 75 |
| UGNX1159 | 207 | UACGUGGAAAUGAACGAGA | 208 | UCUCGUUCAUUUCCACGUA | 69 |
| UGNX1160 | 209 | ACGUGGAAAUGAACGAGAG | 210 | CUCUCGUUCAUUUCCACGU | 70 |
| UGNX1161 | 211 | CGUGGAAAUGAACGAGAGC | 212 | GCUCUCGUUCAUUUCCACG | 83 |
| UGNX1162 | 213 | GUGGAAAUGAACGAGAGCC | 214 | GGCUCUCGUUCAUUUCCAC | 50 |
| UGNX1163 | 215 | UGGAAAUGAACGAGAGCCA | 216 | UGGCUCUCGUUCAUUUCCA | 95 |
| UGNX1164 | 217 | GGAAAUGAACGAGAGCCAC | 218 | GUGGCUCUCGUUCAUUUCC | 95 |
| UGNX1165 | 219 | GAAAUGAACGAGAGCCACA | 220 | UGUGGCUCUCGUUCAUUUC | 99 |
| UGNX1166 | 221 | AAAUGAACGAGAGCCACAC | 222 | GUGUGGCUCUCGUUCAUUU | 77 |
| UGNX1167 | 223 | AAUGAACGAGAGCCACACG | 224 | CGUGUGGCUCUCGUUCAUU | 57 |
| UGNX1168 | 225 | AUGAACGAGAGCCACACGC | 226 | GCGUGUGGCUCUCGUUCAU | 27 |
| UGNX1222 | 227 | CCACAGGCAUUGCCUCCUU | 228 | AAGGAGGCAAUGCCUGUGG | 86 |
| UGNX1224 | 229 | ACAGGCAUUGCCUCCUUCA | 230 | UGAAGGAGGCAAUGCCUGU | 85 |
| UGNX1225 | 231 | CAGGCAUUGCCUCCUUCAC | 232 | GUGAAGGAGGCAAUGCCUG | 81 |
| UGNX1226 | 233 | AGGCAUUGCCUCCUUCACG | 234 | CGUGAAGGAGGCAAUGCCU | 67 |
| UGNX1228 | 235 | GCAUUGCCUCCUUCACGGA | 236 | UCCGUGAAGGAGGCAAUGC | 97 |
| UGNX1229 | 237 | CAUUGCCUCCUUCACGGAG | 238 | CUCCGUGAAGGAGGCAAUG | 54 |
| UGNX1230 | 239 | AUUGCCUCCUUCACGGAGA | 240 | UCUCCGUGAAGGAGGCAAU | 11 |
| UGNX1231 | 241 | UUGCCUCCUUCACGGAGAG | 242 | CUCUCCGUGAAGGAGGCAA | 42 |
| UGNX1232 | 243 | UGCCUCCUUCACGGAGAGA | 244 | UCUCUCCGUGAAGGAGGCA | 27 |
| UGNX1254 | 245 | CCUGGCACACUCAAGACUC | 246 | GAGUCUUGAGUGUGCCAGG | 35 |
| UGNX1255 | 247 | CUGGCACACUCAAGACUCC | 248 | GGAGUCUUGAGUGUGCCAG | 69 |
| UGNX1256 | 249 | UGGCACACUCAAGACUCCC | 250 | GGGAGUCUUGAGUGUGCCA | 30 |
| UGNX1257 | 251 | GGCACACUCAAGACUCCCA | 252 | UGGGAGUCUUGAGUGUGCC | 41 |
| UGNX1258 | 253 | GCACACUCAAGACUCCCAC | 254 | GUGGGAGUCUUGAGUGUGC | 39 |
| UGNX1259 | 255 | CACACUCAAGACUCCCACG | 256 | CGUGGGAGUCUUGAGUGUG | −71 |
| UGNX1260 | 257 | ACACUCAAGACUCCCACGG | 258 | CCGUGGGAGUCUUGAGUGU | 16 |
| UGNX1261 | 259 | CACUCAAGACUCCCACGGA | 260 | UCCGUGGGAGUCUUGAGUG | 30 |
| UGNX1262 | 261 | ACUCAAGACUCCCACGGAG | 262 | CUCCGUGGGAGUCUUGAGU | 3 |
| UGNX1264 | 263 | UCAAGACUCCCACGGAGGU | 264 | ACCUCCGUGGGAGUCUUGA | 80 |
| UGNX1265 | 265 | CAAGACUCCCACGGAGGUU | 266 | AACCUCCGUGGGAGUCUUG | −20 |
| UGNX1266 | 267 | AAGACUCCCACGGAGGUUC | 268 | GAACCUCCGUGGGAGUCUU | 44 |
| UGNX1267 | 269 | AGACUCCCACGGAGGUUCA | 270 | UGAACCUCCGUGGGAGUCU | 63 |
| UGNX1269 | 271 | ACUCCCACGGAGGUUCAGU | 272 | ACUGAACCUCCGUGGGAGU | −8 |
| UGNX1270 | 273 | CUCCCACGGAGGUUCAGUU | 274 | AACUGAACCUCCGUGGGAG | 67 |
| UGNX1271 | 275 | UCCCACGGAGGUUCAGUUC | 276 | GAACUGAACCUCCGUGGGA | 13 |
| UGNX1273 | 277 | CCACGGAGGUUCAGUUCCA | 278 | UGGAACUGAACCUCCGUGG | 95 |
| UGNX1274 | 279 | CACGGAGGUUCAGUUCCAC | 280 | GUGGAACUGAACCUCCGUG | 69 |
| UGNX1275 | 281 | ACGGAGGUUCAGUUCCACA | 282 | UGUGGAACUGAACCUCCGU | 68 |
| UGNX1276 | 283 | CGGAGGUUCAGUUCCACAC | 284 | GUGUGGAACUGAACCUCCG | 89 |
| UGNX1277 | 285 | GGAGGUUCAGUUCCACACU | 286 | AGUGUGGAACUGAACCUCC | 72 |
| UGNX1278 | 287 | GAGGUUCAGUUCCACACUC | 288 | GAGUGUGGAACUGAACCUC | 81 |
| UGNX1279 | 289 | AGGUUCAGUUCCACACUCC | 290 | GGAGUGUGGAACUGAACCU | 83 |
| UGNX1280 | 291 | GGUUCAGUUCCACACUCCC | 292 | GGGAGUGUGGAACUGAACC | 53 |
| UGNX1441 | 293 | UUCCUCUUCGUCUCUCCGG | 294 | CCGGAGAGACGAAGAGGAA | 26 |
| UGNX1598 | 295 | GCUCACCGCCAUUCAUGAA | 296 | UUCAUGAAUGGCGGUGAGC | 63 |
| UGNX1600 | 297 | UCACCGCCAUUCAUGAAGG | 298 | CCUUCAUGAAUGGCGGUGA | 82 |
| UGNX1601 | 299 | CACCGCCAUUCAUGAAGGG | 300 | CCCUUCAUGAAUGGCGGUG | 71 |
| UGNX1632 | 301 | UGCCUGUGGGCCUUUACAA | 302 | UUGUAAAGGCCCACAGGCA | 62 |
| UGNX1633 | 303 | GCCUGUGGGCCUUUACAAG | 304 | CUUGUAAAGGCCCACAGGC | 67 |
| UGNX1634 | 305 | CCUGUGGGCCUUUACAAGG | 306 | CCUUGUAAAGGCCCACAGG | 49 |
| UGNX1635 | 307 | CUGUGGGCCUUUACAAGGG | 308 | CCCUUGUAAAGGCCCACAG | 59 |
| UGNX1636 | 309 | UGUGGGCCUUUACAAGGGC | 310 | GCCCUUGUAAAGGCCCACA | 30 |
| UGNX1759 | 311 | CGUCCGUCCGUGAAAUUCC | 312 | GGAAUUUCACGGACGGACG | 85 |
| UGNX1760 | 313 | GUCCGUCCGUGAAAUUCCG | 314 | CGGAAUUUCACGGACGGAC | 92 |
| UGNX1761 | 315 | UCCGUCCGUGAAAUUCCGG | 316 | CCGGAAUUUCACGGACGGA | 35 |
| UGNX1856 | 317 | GACGGAGACUCGUUUGGAC | 318 | GUCCAAACGAGUCUCCGUC | 81 |
| UGNX1857 | 319 | ACGGAGACUCGUUUGGACC | 320 | GGUCCAAACGAGUCUCCGU | 63 |
| UGNX1899 | 321 | AGCCUGCUUUGAGCGGAAC | 322 | GUUCCGCUCAAAGCAGGCU | 29 |
| UGNX1904 | 323 | GCUUUGAGCGGAACCCGUA | 324 | UACGGGUUCCGCUCAAAGC | 96 |
| UGNX1905 | 325 | CUUUGAGCGGAACCCGUAC | 326 | GUACGGGUUCCGCUCAAAG | −5 |
| UGNX1906 | 327 | UUUGAGCGGAACCCGUACC | 328 | GGUACGGGUUCCGCUCAAA | −15 |
| UGNX1975 | 329 | CCCAGGGUCCAGAUUUGGU | 330 | ACCAAAUCUGGACCCUGGG | 11 |
| UGNX1976 | 331 | CCAGGGUCCAGAUUUGGUU | 332 | AACCAAAUCUGGACCCUGG | 99 |
| UGNX1977 | 333 | CAGGGUCCAGAUUUGGUUU | 334 | AAACCAAAUCUGGACCCUG | 22 |
| UGNX1978 | 335 | AGGGUCCAGAUUUGGUUUC | 336 | GAAACCAAAUCUGGACCCU | −21 |
| UGNX1979 | 337 | GGGUCCAGAUUUGGUUUCA | 338 | UGAAACCAAAUCUGGACCC | 80 |
| UGNX1980 | 339 | GGUCCAGAUUUGGUUUCAG | 340 | CUGAAACCAAAUCUGGACC | 99 |
| UGNX1981 | 341 | GUCCAGAUUUGGUUUCAGA | 342 | UCUGAAACCAAAUCUGGAC | 99 |
| UGNX1982 | 343 | UCCAGAUUUGGUUUCAGAA | 344 | UUCUGAAACCAAAUCUGGA | 99 |
| UGNX1983 | 345 | CCAGAUUUGGUUUCAGAAU | 346 | AUUCUGAAACCAAAUCUGG | 79 |
| UGNX1984 | 347 | CAGAUUUGGUUUCAGAAUG | 348 | CAUUCUGAAACCAAAUCUG | 98 |
| UGNX1985 | 349 | AGAUUUGGUUUCAGAAUGA | 350 | UCAUUCUGAAACCAAAUCU | 99 |
| UGNX1986 | 351 | GAUUUGGUUUCAGAAUGAG | 352 | CUCAUUCUGAAACCAAAUC | 96 |
| UGNX1987 | 353 | AUUUGGUUUCAGAAUGAGA | 354 | UCUCAUUCUGAAACCAAAU | 96 |
| UGNX1988 | 355 | UUUGGUUUCAGAAUGAGAG | 356 | CUCUCAUUCUGAAACCAAA | 64 |
| UGNX1989 | 357 | UUGGUUUCAGAAUGAGAGG | 358 | CCUCUCAUUCUGAAACCAA | 62 |
| UGNX1990 | 359 | UGGUUUCAGAAUGAGAGGU | 360 | ACCUCUCAUUCUGAAACCA | 74 |
| UGNX1991 | 361 | GGUUUCAGAAUGAGAGGUC | 362 | GACCUCUCAUUCUGAAACC | 99 |
| UGNX1992 | 363 | GUUUCAGAAUGAGAGGUCA | 364 | UGACCUCUCAUUCUGAAAC | 97 |
| UGNX1993 | 365 | UUUCAGAAUGAGAGGUCAC | 366 | GUGACCUCUCAUUCUGAAA | 60 |
| UGNX1994 | 367 | UUCAGAAUGAGAGGUCACG | 368 | CGUGACCUCUCAUUCUGAA | 44 |
| UGNX1995 | 369 | UCAGAAUGAGAGGUCACGC | 370 | GCGUGACCUCUCAUUCUGA | −48 |
| UGNX1996 | 371 | CAGAAUGAGAGGUCACGCC | 372 | GGCGUGACCUCUCAUUCUG | 84 |
| UGNX1997 | 373 | AGAAUGAGAGGUCACGCCA | 374 | UGGCGUGACCUCUCAUUCU | 15 |
| UGNX1998 | 375 | GAAUGAGAGGUCACGCCAG | 376 | CUGGCGUGACCUCUCAUUC | 70 |
| UGNX1999 | 377 | AAUGAGAGGUCACGCCAGC | 378 | GCUGGCGUGACCUCUCAUU | 95 |
| UGNX2000 | 379 | AUGAGAGGUCACGCCAGCU | 380 | AGCUGGCGUGACCUCUCAU | 33 |
| UGNX2117 | 381 | UGCUCCUCCGAGCCUUUGA | 382 | UCAAAGGCUCGGAGGAGCA | 80 |
| UGNX2120 | 383 | UCCUCCGAGCCUUUGAGAA | 384 | UUCUCAAAGGCUCGGAGGA | 18 |
| UGNX2121 | 385 | CCUCCGAGCCUUUGAGAAG | 386 | CUUCUCAAAGGCUCGGAGG | 96 |
| UGNX2122 | 387 | CUCCGAGCCUUUGAGAAGG | 388 | CCUUCUCAAAGGCUCGGAG | 87 |
| UGNX2123 | 389 | UCCGAGCCUUUGAGAAGGA | 390 | UCCUUCUCAAAGGCUCGGA | 48 |
| UGNX2124 | 391 | CCGAGCCUUUGAGAAGGAU | 392 | AUCCUUCUCAAAGGCUCGG | 76 |
| UGNX2125 | 393 | CGAGCCUUUGAGAAGGAUC | 394 | GAUCCUUCUCAAAGGCUCG | 92 |
| UGNX2126 | 395 | GAGCCUUUGAGAAGGAUCG | 396 | CGAUCCUUCUCAAAGGCUC | 46 |
| UGNX2127 | 397 | AGCCUUUGAGAAGGAUCGC | 398 | GCGAUCCUUCUCAAAGGCU | 8 |
| UGNX2128 | 399 | GCCUUUGAGAAGGAUCGCU | 400 | AGCGAUCCUUCUCAAAGGC | 97 |
| UGNX2129 | 401 | CCUUUGAGAAGGAUCGCUU | 402 | AAGCGAUCCUUCUCAAAGG | 98 |
| UGNX2130 | 403 | CUUUGAGAAGGAUCGCUUU | 404 | AAAGCGAUCCUUCUCAAAG | 98 |
| UGNX2131 | 405 | UUUGAGAAGGAUCGCUUUC | 406 | GAAAGCGAUCCUUCUCAAA | 10 |
| UGNX2132 | 407 | UUGAGAAGGAUCGCUUUCC | 408 | GGAAAGCGAUCCUUCUCAA | 35 |
| UGNX2133 | 409 | UGAGAAGGAUCGCUUUCCA | 410 | UGGAAAGCGAUCCUUCUCA | 93 |
| UGNX2134 | 411 | GAGAAGGAUCGCUUUCCAG | 412 | CUGGAAAGCGAUCCUUCUC | −150 |
| UGNX2135 | 413 | AGAAGGAUCGCUUUCCAGG | 414 | CCUGGAAAGCGAUCCUUCU | −180 |
| UGNX2136 | 415 | GAAGGAUCGCUUUCCAGGC | 416 | GCCUGGAAAGCGAUCCUUC | 31 |
| UGNX2137 | 417 | AAGGAUCGCUUUCCAGGCA | 418 | UGCCUGGAAAGCGAUCCUU | 30 |
| UGNX2138 | 419 | AGGAUCGCUUUCCAGGCAU | 420 | AUGCCUGGAAAGCGAUCCU | −16 |
| UGNX2139 | 421 | GGAUCGCUUUCCAGGCAUC | 422 | GAUGCCUGGAAAGCGAUCC | 98 |
| UGNX2140 | 423 | GAUCGCUUUCCAGGCAUCG | 424 | CGAUGCCUGGAAAGCGAUC | 35 |
| UGNX2194 | 425 | CCGGAGUCCAGGAUUCAGA | 426 | UCUGAAUCCUGGACUCCGG | 99 |
| UGNX2195 | 427 | CGGAGUCCAGGAUUCAGAU | 428 | AUCUGAAUCCUGGACUCCG | 17 |
| UGNX2196 | 429 | GGAGUCCAGGAUUCAGAUC | 430 | GAUCUGAAUCCUGGACUCC | −41 |
| UGNX2197 | 431 | GAGUCCAGGAUUCAGAUCU | 432 | AGAUCUGAAUCCUGGACUC | 4 |
| UGNX2198 | 433 | AGUCCAGGAUUCAGAUCUG | 434 | CAGAUCUGAAUCCUGGACU | 89 |
| UGNX2199 | 435 | GUCCAGGAUUCAGAUCUGG | 436 | CCAGAUCUGAAUCCUGGAC | 45 |
| UGNX2200 | 437 | UCCAGGAUUCAGAUCUGGU | 438 | ACCAGAUCUGAAUCCUGGA | 26 |
| UGNX2201 | 439 | CCAGGAUUCAGAUCUGGUU | 440 | AACCAGAUCUGAAUCCUGG | −44 |
| UGNX2202 | 441 | CAGGAUUCAGAUCUGGUUU | 442 | AAACCAGAUCUGAAUCCUG | −27 |
| UGNX2203 | 443 | AGGAUUCAGAUCUGGUUUC | 444 | GAAACCAGAUCUGAAUCCU | −43 |
| UGNX2204 | 445 | GGAUUCAGAUCUGGUUUCA | 446 | UGAAACCAGAUCUGAAUCC | 96 |
| UGNX2205 | 447 | GAUUCAGAUCUGGUUUCAG | 448 | CUGAAACCAGAUCUGAAUC | 98 |
| UGNX2206 | 449 | AUUCAGAUCUGGUUUCAGA | 450 | UCUGAAACCAGAUCUGAAU | 97 |
| UGNX2207 | 451 | UUCAGAUCUGGUUUCAGAA | 452 | UUCUGAAACCAGAUCUGAA | 92 |
| UGNX2208 | 453 | UCAGAUCUGGUUUCAGAAU | 454 | AUUCUGAAACCAGAUCUGA | 56 |
| UGNX2209 | 455 | CAGAUCUGGUUUCAGAAUC | 456 | GAUUCUGAAACCAGAUCUG | 98 |
| UGNX2210 | 457 | AGAUCUGGUUUCAGAAUCG | 458 | CGAUUCUGAAACCAGAUCU | 5 |
| UGNX2211 | 459 | GAUCUGGUUUCAGAAUCGA | 460 | UCGAUUCUGAAACCAGAUC | 96 |
| UGNX2212 | 461 | AUCUGGUUUCAGAAUCGAA | 462 | UUCGAUUCUGAAACCAGAU | 99 |
| UGNX2213 | 463 | UCUGGUUUCAGAAUCGAAG | 464 | CUUCGAUUCUGAAACCAGA | −11 |
| UGNX2214 | 465 | CUGGUUUCAGAAUCGAAGG | 466 | CCUUCGAUUCUGAAACCAG | 85 |
| UGNX2215 | 467 | UGGUUUCAGAAUCGAAGGG | 468 | CCCUUCGAUUCUGAAACCA | 9 |
| UGNX2216 | 469 | GGUUUCAGAAUCGAAGGGC | 470 | GCCCUUCGAUUCUGAAACC | 6 |
| UGNX2217 | 471 | GUUUCAGAAUCGAAGGGCC | 472 | GGCCCUUCGAUUCUGAAAC | 52 |
| UGNX2218 | 473 | UUUCAGAAUCGAAGGGCCA | 474 | UGGCCCUUCGAUUCUGAAA | 17 |
| UGNX2219 | 475 | UUCAGAAUCGAAGGGCCAG | 476 | CUGGCCCUUCGAUUCUGAA | −47 |
| UGNX2220 | 477 | UCAGAAUCGAAGGGCCAGG | 478 | CCUGGCCCUUCGAUUCUGA | −40 |
| UGNX2222 | 479 | AGAAUCGAAGGGCCAGGCA | 480 | UGCCUGGCCCUUCGAUUCU | 17 |
| UGNX2951 | 481 | AGGCGCAACCUCUCCUAGA | 482 | UCUAGGAGAGGUUGCGCCU | 98 |
| UGNX2953 | 483 | GCGCAACCUCUCCUAGAAA | 484 | UUUCUAGGAGAGGUUGCGC | −11 |
| UGNX2954 | 485 | CGCAACCUCUCCUAGAAAC | 486 | GUUUCUAGGAGAGGUUGCG | −28 |
| UGNX2955 | 487 | GCAACCUCUCCUAGAAACG | 488 | CGUUUCUAGGAGAGGUUGC | 10 |
| UGNX2956 | 489 | CAACCUCUCCUAGAAACGG | 490 | CCGUUUCUAGGAGAGGUUG | 16 |
| UGNX2957 | 491 | AACCUCUCCUAGAAACGGA | 492 | UCCGUUUCUAGGAGAGGUU | 52 |
| UGNX2958 | 493 | ACCUCUCCUAGAAACGGAG | 494 | CUCCGUUUCUAGGAGAGGU | 4 |
| UGNX2959 | 495 | CCUCUCCUAGAAACGGAGG | 496 | CCUCCGUUUCUAGGAGAGG | −53 |
| UGNX2960 | 497 | CUCUCCUAGAAACGGAGGC | 498 | GCCUCCGUUUCUAGGAGAG | −17 |
| UGNX2961 | 499 | UCUCCUAGAAACGGAGGCC | 500 | GGCCUCCGUUUCUAGGAGA | −554 |
| UGNX3029 | 501 | UCAGCGAGGAAGAAUACCG | 502 | CGGUAUUCUUCCUCGCUGA | −40 |
| UGNX3030 | 503 | CAGCGAGGAAGAAUACCGG | 504 | CCGGUAUUCUUCCUCGCUG | 86 |
| UGNX3031 | 505 | AGCGAGGAAGAAUACCGGG | 506 | CCCGGUAUUCUUCCUCGCU | 80 |
| UGNX3033 | 507 | CGAGGAAGAAUACCGGGCU | 508 | AGCCCGGUAUUCUUCCUCG | 90 |
| UGNX3034 | 509 | GAGGAAGAAUACCGGGCUC | 510 | GAGCCCGGUAUUCUUCCUC | 80 |
| UGNX3035 | 511 | AGGAAGAAUACCGGGCUCU | 512 | AGAGCCCGGUAUUCUUCCU | 91 |
| UGNX3036 | 513 | GGAAGAAUACCGGGCUCUG | 514 | CAGAGCCCGGUAUUCUUCC | −78 |
| UGNX3037 | 515 | GAAGAAUACCGGGCUCUGC | 516 | GCAGAGCCCGGUAUUCUUC | −101 |
| UGNX3038 | 517 | AAGAAUACCGGGCUCUGCU | 518 | AGCAGAGCCCGGUAUUCUU | −280 |
| UGNX3039 | 519 | AGAAUACCGGGCUCUGCUG | 520 | CAGCAGAGCCCGGUAUUCU | −50 |
| UGNX3041 | 521 | AAUACCGGGCUCUGCUGGA | 522 | UCCAGCAGAGCCCGGUAUU | −27 |
| UGNX3049 | 523 | GCUCUGCUGGAGGAGCUUU | 524 | AAAGCUCCUCCAGCAGAGC | −66 |
| UGNX3050 | 525 | CUCUGCUGGAGGAGCUUUA | 526 | UAAAGCUCCUCCAGCAGAG | 87 |
| UGNX3051 | 527 | UCUGCUGGAGGAGCUUUAG | 528 | CUAAAGCUCCUCCAGCAGA | −52 |
| UGNX3052 | 529 | CUGCUGGAGGAGCUUUAGG | 530 | CCUAAAGCUCCUCCAGCAG | −57 |
| UGNX3053 | 531 | UGCUGGAGGAGCUUUAGGA | 532 | UCCUAAAGCUCCUCCAGCA | −18 |
| UGNX3054 | 533 | GCUGGAGGAGCUUUAGGAC | 534 | GUCCUAAAGCUCCUCCAGC | −60 |
| UGNX3055 | 535 | CUGGAGGAGCUUUAGGACG | 536 | CGUCCUAAAGCUCCUCCAG | 28 |
| UGNX3056 | 537 | UGGAGGAGCUUUAGGACGC | 538 | GCGUCCUAAAGCUCCUCCA | 31 |
| UGNX3193 | 539 | CCUGGGAUUCCUGCCUUCU | 540 | AGAAGGCAGGAAUCCCAGG | −84 |
| UGNX3195 | 541 | UGGGAUUCCUGCCUUCUAG | 542 | CUAGAAGGCAGGAAUCCCA | −36 |
| UGNX3196 | 543 | GGGAUUCCUGCCUUCUAGG | 544 | CCUAGAAGGCAGGAAUCCC | 0 |
| UGNX3197 | 545 | GGAUUCCUGCCUUCUAGGU | 546 | ACCUAGAAGGCAGGAAUCC | 2 |
| UGNX3198 | 547 | GAUUCCUGCCUUCUAGGUC | 548 | GACCUAGAAGGCAGGAAUC | −6 |
| UGNX3199 | 549 | AUUCCUGCCUUCUAGGUCU | 550 | AGACCUAGAAGGCAGGAAU | 20 |
| UGNX3200 | 551 | UUCCUGCCUUCUAGGUCUA | 552 | UAGACCUAGAAGGCAGGAA | −39 |
| UGNX3201 | 553 | UCCUGCCUUCUAGGUCUAG | 554 | CUAGACCUAGAAGGCAGGA | −39 |
| UGNX3202 | 555 | CCUGCCUUCUAGGUCUAGG | 556 | CCUAGACCUAGAAGGCAGG | −8 |
| UGNX3203 | 557 | CUGCCUUCUAGGUCUAGGC | 558 | GCCUAGACCUAGAAGGCAG | −48 |
| UGNX3204 | 559 | UGCCUUCUAGGUCUAGGCC | 560 | GGCCUAGACCUAGAAGGCA | −31 |
| UGNX3208 | 561 | UUCUAGGUCUAGGCCCGGU | 562 | ACCGGGCCUAGACCUAGAA | −71 |
| UGNX3241 | 563 | CGCGGAGAACUGCCAUUCU | 564 | AGAAUGGCAGUUCUCCGCG | 92 |
| UGNX3243 | 565 | CGGAGAACUGCCAUUCUUU | 566 | AAAGAAUGGCAGUUCUCCG | −121 |
| UGNX3244 | 567 | GGAGAACUGCCAUUCUUUC | 568 | GAAAGAAUGGCAGUUCUCC | −43 |
| UGNX3245 | 569 | GAGAACUGCCAUUCUUUCC | 570 | GGAAAGAAUGGCAGUUCUC | 59 |
| UGNX3246 | 571 | AGAACUGCCAUUCUUUCCU | 572 | AGGAAAGAAUGGCAGUUCU | 10 |
| UGNX3247 | 573 | GAACUGCCAUUCUUUCCUG | 574 | CAGGAAAGAAUGGCAGUUC | −92 |
| UGNX3248 | 575 | AACUGCCAUUCUUUCCUGG | 576 | CCAGGAAAGAAUGGCAGUU | −70 |
| UGNX3249 | 577 | ACUGCCAUUCUUUCCUGGG | 578 | CCCAGGAAAGAAUGGCAGU | −25 |
| UGNX3250 | 579 | CUGCCAUUCUUUCCUGGGC | 580 | GCCCAGGAAAGAAUGGCAG | −22 |
| UGNX3251 | 581 | UGCCAUUCUUUCCUGGGCA | 582 | UGCCCAGGAAAGAAUGGCA | −65 |
| UGNX3252 | 583 | GCCAUUCUUUCCUGGGCAU | 584 | AUGCCCAGGAAAGAAUGGC | 82 |
| UGNX3253 | 585 | CCAUUCUUUCCUGGGCAUC | 586 | GAUGCCCAGGAAAGAAUGG | 2 |
| UGNX3254 | 587 | CAUUCUUUCCUGGGCAUCC | 588 | GGAUGCCCAGGAAAGAAUG | −390 |
| UGNX3255 | 589 | AUUCUUUCCUGGGCAUCCC | 590 | GGGAUGCCCAGGAAAGAAU | −110 |
| UGNX3256 | 591 | UUCUUUCCUGGGCAUCCCG | 592 | CGGGAUGCCCAGGAAAGAA | −40 |

TABLE 3
% Inhibition in 54-2 Cells

| | SEQ | | SEQ | | % |
|---|---|---|---|---|---|
| SiRNA | ID | | ID | | inhibition |
| name | NO. | sense | NO: | antisense | in 54-2 |
| UGNX394 | 1 | GGCAGAGAUGGAGAGAGGA | 2 | UCCUCUCUCCAUCUCUGCC | 29 |
| UGNX482 | 3 | AGGGAGGAACGGAGGGAAA | 4 | UUUCCCUCCGUUCCUCCCU | 30 |
| UGNX554 | 5 | GAGGAAGGCAGGGAGGAAA | 6 | UUUCCUCCCUGCCUUCCUC | −11 |
| UGNX722 | 7 | CGGUUUCCUCCGGGACAAA | 8 | UUUGUCCCGGAGGAAACCG | 19 |
| UGNX779 | 9 | CGGUUCACAGACCGCACAU | 10 | AUGUGCGGUCUGUGAACCG | 38 |
| UGNX864 | 11 | GACGACGGAGGCGUGAUUU | 12 | AAAUCACGCCUCCGUCGUC | 19 |
| UGNX916 | 13 | GCCUGUUGCUCACGUCUCU | 14 | AGAGACGUGAGCAACAGGC | 41 |
| UGNX950 | 15 | CUGGCCAUGCCGACUGUUU | 16 | AAACAGUCGGCAUGGCCAG | −57 |
| UGNX991 | 17 | CCGGAAACAUGCAGGGAAG | 18 | CUUCCCUGCAUGUUUCCGG | 29 |
| UGNX1032 | 19 | UUCGCUCUCCUUGCCAGGU | 20 | ACCUGGCAAGGAGAGCGAA | 31 |
| UGNX1094 | 21 | GGAAUCCAUCGUCAGGCCA | 22 | UGGCCUGACGAUGGAUUCC | 50 |
| UGNX1142 | 23 | GUCUCGCUCUGGUCUUCUA | 24 | UAGAAGACCAGAGCGAGAC | 5 |
| UGNX1223 | 25 | CACAGGCAUUGCCUCCUUC | 26 | GAAGGAGGCAAUGCCUGUG | −92 |
| UGNX1253 | 27 | GCCUGGCACACUCAAGACU | 28 | AGUCUUGAGUGUGCCAGGC | −19 |
| UGNX1311 | 29 | AGGCUGGUUUCUCCCUGCU | 30 | AGCAGGGAGAAACCAGCCU | 41 |
| UGNX1440 | 31 | CUUCCUCUUCGUCUCUCCG | 32 | CGGAGAGACGAAGAGGAAG | 44 |
| UGNX1599 | 33 | CUCACCGCCAUUCAUGAAG | 34 | CUUCAUGAAUGGCGGUGAG | 68 |
| UGNX1631 | 35 | CUGCCUGUGGGCCUUUACA | 36 | UGUAAAGGCCCACAGGCAG | 68 |
| UGNX1758 | 37 | GCGUCCGUCCGUGAAAUUC | 38 | GAAUUUCACGGACGGACGC | 90 |
| UGNX1855 | 39 | CGACGGAGACUCGUUUGGA | 40 | UCCAAACGAGUCUCCGUCG | 82 |
| UGNX1898 | 41 | GAGCCUGCUUUGAGCGGAA | 42 | UUCCGCUCAAAGCAGGCUC | 72 |
| UGNX1972 | 43 | GAGCCCAGGGUCCAGAUUU | 44 | AAAUCUGGACCCUGGGCUC | 50 |
| UGNX2119 | 45 | CUCCUCCGAGCCUUUGAGA | 46 | UCUCAAAGGCUCGGAGGAG | 64 |
| UGNX2192 | 47 | UCCCGGAGUCCAGGAUUCA | 48 | UGAAUCCUGGACUCCGGGA | 69 |
| UGNX2909 | 49 | UGCUGCUGGAUGAGCUCCU | 50 | AGGAGCUCAUCCAGCAGCA | 43 |
| UGNX2952 | 51 | GGCGCAACCUCUCCUAGAA | 52 | UUCUAGGAGAGGUUGCGCC | 81 |
| UGNX3028 | 53 | CUCAGCGAGGAAGAAUACC | 54 | GGUAUUCUUCCUCGCUGAG | 41 |
| UGNX3194 | 55 | CUGGGAUUCCUGCCUUCUA | 56 | UAGAAGGCAGGAAUCCCAG | 67 |
| UGNX3242 | 57 | GCGGAGAACUGCCAUUCUU | 58 | AAGAAUGGCAGUUCUCCGC | 87 |
| UGNX1759 | 311 | CGUCCGUCCGUGAAAUUCC | 312 | GGAAUUUCACGGACGGACG | 85 |
| UGNX1856 | 317 | GACGGAGACUCGUUUGGAC | 318 | GUCCAAACGAGUCUCCGUC | 5 |
| UGNX1857 | 319 | ACGGAGACUCGUUUGGACC | 320 | GGUCCAAACGAGUCUCCGU | −9 |
| UGNX1899 | 321 | AGCCUGCUUUGAGCGGAAC | 322 | GUUCCGCUCAAAGCAGGCU | 10 |
| UGNX1905 | 325 | CUUUGAGCGGAACCCGUAC | 326 | GUACGGGUUCCGCUCAAAG | −79 |
| UGNX1906 | 327 | UUUGAGCGGAACCCGUACC | 328 | GGUACGGGUUCCGCUCAAA | −291 |
| UGNX1975 | 329 | CCCAGGGUCCAGAUUUGGU | 330 | ACCAAAUCUGGACCCUGGG | 4 |
| UGNX1977 | 333 | CAGGGUCCAGAUUUGGUUU | 334 | AAACCAAAUCUGGACCCUG | −23 |
| UGNX1978 | 335 | AGGGUCCAGAUUUGGUUUC | 336 | GAAACCAAAUCUGGACCCU | 5 |
| UGNX1979 | 337 | GGGUCCAGAUUUGGUUUCA | 338 | UGAAACCAAAUCUGGACCC | 66 |
| UGNX1983 | 345 | CCAGAUUUGGUUUCAGAAU | 346 | AUUCUGAAACCAAAUCUGG | −9 |
| UGNX1986 | 351 | GAUUUGGUUUCAGAAUGAG | 352 | CUCAUUCUGAAACCAAAUC | 72 |
| UGNX1988 | 355 | UUUGGUUUCAGAAUGAGAG | 356 | CUCUCAUUCUGAAACCAAA | 51 |
| UGNX1989 | 357 | UUGGUUUCAGAAUGAGAGG | 358 | CCUCUCAUUCUGAAACCAA | 42 |
| UGNX1990 | 359 | UGGUUUCAGAAUGAGAGGU | 360 | ACCUCUCAUUCUGAAACCA | 19 |
| UGNX1991 | 361 | GGUUUCAGAAUGAGAGGUC | 362 | GACCUCUCAUUCUGAAACC | 10 |
| UGNX1993 | 365 | UUUCAGAAUGAGAGGUCAC | 366 | GUGACCUCUCAUUCUGAAA | 37 |
| UGNX1994 | 367 | UUCAGAAUGAGAGGUCACG | 368 | CGUGACCUCUCAUUCUGAA | 65 |
| UGNX1995 | 369 | UCAGAAUGAGAGGUCACGC | 370 | GCGUGACCUCUCAUUCUGA | −233 |
| UGNX1996 | 371 | CAGAAUGAGAGGUCACGCC | 372 | GGCGUGACCUCUCAUUCUG | 95 |
| UGNX1997 | 373 | AGAAUGAGAGGUCACGCCA | 374 | UGGCGUGACCUCUCAUUCU | 1 |
| UGNX1998 | 375 | GAAUGAGAGGUCACGCCAG | 376 | CUGGCGUGACCUCUCAUUC | 70 |
| UGNX1999 | 377 | AAUGAGAGGUCACGCCAGC | 378 | GCUGGCGUGACCUCUCAUU | 23 |
| UGNX2000 | 379 | AUGAGAGGUCACGCCAGCU | 380 | AGCUGGCGUGACCUCUCAU | 40 |
| UGNX2117 | 381 | UGCUCCUCCGAGCCUUUGA | 382 | UCAAAGGCUCGGAGGAGCA | 28 |
| UGNX2120 | 383 | UCCUCCGAGCCUUUGAGAA | 384 | UUCUCAAAGGCUCGGAGGA | 5 |
| UGNX2123 | 389 | UCCGAGCCUUUGAGAAGGA | 390 | UCCUUCUCAAAGGCUCGGA | −5 |
| UGNX2124 | 391 | CCGAGCCUUUGAGAAGGAU | 392 | AUCCUUCUCAAAGGCUCGG | 64 |
| UGNX2125 | 393 | CGAGCCUUUGAGAAGGAUC | 394 | GAUCCUUCUCAAAGGCUCG | 33 |
| UGNX2126 | 395 | GAGCCUUUGAGAAGGAUCG | 396 | CGAUCCUUCUCAAAGGCUC | −8 |
| UGNX2127 | 397 | AGCCUUUGAGAAGGAUCGC | 398 | GCGAUCCUUCUCAAAGGCU | 15 |
| UGNX2131 | 405 | UUUGAGAAGGAUCGCUUUC | 406 | GAAAGCGAUCCUUCUCAAA | 3 |
| UGNX2132 | 407 | UUGAGAAGGAUCGCUUUCC | 408 | GGAAAGCGAUCCUUCUCAA | 47 |

### Example 3: Inhibition of DUX4 with Antisense siRNA

Further dose response studies were conducted using the concentrations of 10 nM, 2 nM and 0.4 nM. Transfections of siRNAs into FSHD1 and FSHD2 myoblasts were carried out using Lipofectamine RNAiMAX (Invitrogen) as above. Briefly, cells were seeded at 1×10⁵ cells/well in 1 ml regular growth medium in 12-well culture plates. Approximately 20 hours later, cells were transfected by first diluting 2 μl Lipofectamine RNAiMAX and 10, 2 or 0.4 pmol of either gene-specific siRNAs or a scrambled non-silencing control siRNA in 100 μl Opti-MEM Reduced Serum Medium to create lipofectamine/siRNA complexes. The lipofectamine/siRNA complexes were then added dropwise to one well of the culture plate containing myoblasts. 24 hours following transfection, cells were incubated in differentiation medium for 40 hours to induce differentiation into myotubes and harvested for total RNA analysis.

For determination of the 50% inhibitory concentration (IC50) for each siRNA, seven-point concentration response curves were generated by first creating 3-fold serial dilutions of siRNAs from concentrated stocks in water in 96-well plates. Transfection then proceeded as above. IC50s were determined by nonlinear regression using a four-parameter logistic equation (GraphPad Prism Software Inc., San Diego, CA; http://www.graphpad.com). Data is presented as IC50s with two significant digits. Data from the dose response and IC50 studies are summarized are in Table 4.

TABLE 4
Dose Response and IC50 Studies

| | SEQ | | SEQ | | MB200 | | | 54-2 | | | | |
|---|---|---|---|---|---|---|---|---|---|---|---|---|
| SiRNA | ID | | ID | anti- | 10 | 2 | 0.4 | 10 | 2 | 0.4 | MB200 | 54-2 |
| name | NO. | sense | NO: | sense | nM | nM | nM | nM | nM | nM | IC50 | IC50 |
| UGNX | 37 | GCGUCCG | 38 | GAAUU | 95 | 52 | 6 | 95 | 32 | 15 | | |
| 1758 | | UCCGUGA | | UCACGG | | | | | | | | |
| | | AAUUC | | ACGGAC | | | | | | | | |
| | | | | GC | | | | | | | | |
| UGNX | 39 | CGACGGA | 40 | UCCAAA | 92 | 38 | 29 | 87 | 35 | 22 | | |
| 1855 | | GACUCGU | | CGAGUC | | | | | | | | |
| | | UUGGA | | UCCGUC | | | | | | | | |
| | | | | G | | | | | | | | |
| UGNX | 41 | GAGCCUG | 42 | UUCCGC | 91 | 63 | 29 | 91 | 48 | 23 | | |
| 1898 | | CUUUGAG | | UCAAAG | | | | | | | | |
| | | CGGAA | | CAGGCU | | | | | | | | |
| | | | | C | | | | | | | | |
| UGNX | 51 | GGCGCAA | 52 | UUCUAG | 94 | 65 | 15 | 93 | 62 | 32 | | |
| 2952 | | CCUCUCC | | GAGAG | | | | | | | | |
| | | UAGAA | | GUUGCG | | | | | | | | |
| | | | | CC | | | | | | | | |
| UGNX | 55 | CUGGGAU | 56 | UAGAA | 76 | 21 | 10 | 77 | 38 | 24 | | |
| 3194 | | UCCUGCC | | GGCAGG | | | | | | | | |
| | | UUCUA | | AAUCCC | | | | | | | | |
| | | | | AG | | | | | | | | |
| UGNX | 57 | GCGGAGA | 58 | AAGAA | 96 | 89 | 27 | 95 | 77 | 25 | | |
| 3242 | | ACUGCCA | | UGGCAG | | | | | | | | |
| | | UUCUU | | | | | | | | | | |
| | | | | UUCUCC | | | | | | | | |
| | | | | GC | | | | | | | | |
| UGNX | 73 | UGGAGAG | 74 | UCUCCC | 87 | 9 | 31 | 77 | 48 | 38 | | |
| 402 | | AGGAACG | | GUUCCU | | | | | | | | |
| | | GGAGA | | CUCUCC | | | | | | | | |
| | | | | A | | | | | | | | |
| UGNX | 77 | GAGAGGA | 78 | UAGGUC | 92 | 79 | 52 | 80 | 59 | 43 | | |
| 406 | | ACGGGAG | | UCCCGU | | | | | | | | |
| | | ACCUA | | UCCUCU | | | | | | | | |
| | | | | C | | | | | | | | |
| UGNX | 93 | GAACGGA | 94 | UCUGUC | 95 | 47 | 33 | 90 | 69 | 38 | | |
| 488 | | GGGAAAG | | UUUCCC | | | | | | | | |
| | | ACAGA | | UCCGUU | | | | | | | | |
| | | | | C | | | | | | | | |
| UGNX | 113 | GUUCACA | 114 | GGAUG | 95 | 50 | 35 | 60 | 26 | 39 | | |
| 781 | | GACCGCA | | UGCGGU | | | | | | | | |
| | | CAUCC | | CUGUGA | | | | | | | | |
| | | | | AC | | | | | | | | |
| UGNX | 115 | UUCACAG | 116 | GGGAU | 87 | 33 | 31 | 55 | 34 | 34 | | |
| 782 | | ACCGCAC | | GUGCGG | | | | | | | | |
| | | AUCCC | | UCUGUG | | | | | | | | |
| | | | | AA | | | | | | | | |
| UGNX | 129 | UGUUUGC | 130 | AGAGCU | 90 | 42 | 40 | 39 | 47 | 36 | | |
| 964 | | UCCCGGA | | CCGGGA | | | | | | | | |
| | | GCUCU | | GCAAAC | | | | | | | | |
| | | | | A | | | | | | | | |
| UGNX | 131 | CCCGGAA | 132 | UUCCCU | 72 | 51 | 38 | 80 | 46 | 38 | | |
| 990 | | ACAUGCA | | GCAUGU | | | | | | | | |
| | | GGGAA | | UUCCGG | | | | | | | | |
| | | | | G | | | | | | | | |
| UGNX | 171 | AUCCAUC | 172 | UGAUG | 98 | 68 | −2 | 70 | 51 | 35 | | |
| 1097 | | GUCAGGC | | GCCUGA | | | | | | | | |
| | | CAUCA | | CGAUGG | | | | | | | | |
| | | | | AU | | | | | | | | |
| UGNX | 181 | CGCUCUG | 182 | CACGUA | 77 | 34 | 34 | 78 | 60 | 32 | | |
| 1146 | | GUCUUCU | | GAAGAC | | | | | | | | |
| | | ACGUG | | CAGAGC | | | | | | | | |
| | | | | G | | | | | | | | |
| UGNX | 187 | UCUGGUC | 188 | UUCCAC | 78 | 73 | 15 | 37 | 19 | −8 | | |
| 1149 | | UUCUACG | | GUAGA | | | | | | | | |
| | | UGGAA | | AGACCA | | | | | | | | |
| | | | | GA | | | | | | | | |
| UGNX | 215 | UGGAAAU | 216 | UGGCUC | 98 | 68 | 17 | 86 | 70 | 48 | | |
| 1163 | | GAACGAG | | UCGUUC | | | | | | | | |
| | | AGCCA | | AUUUCC | | | | | | | | |
| | | | | A | | | | | | | | |
| UGNX | 217 | GGAAAUG | 218 | GUGGCU | 96 | 57 | 2 | 91 | 64 | 47 | | |
| 1164 | | AACGAGA | | CUCGUU | | | | | | | | |
| | | GCCAC | | CAUUUC | | | | | | | | |
| | | | | C | | | | | | | | |
| UGNX | 219 | GAAAUGA | 220 | UGUGGC | 99 | 88 | 71 | 93 | 67 | 52 | | |
| 1165 | | ACGAGAG | | UCUCGU | | | | | | | | |
| | | CCACA | | UCAUUU | | | | | | | | |
| | | | | C | | | | | | | | |
| UGNX | 235 | GCAUUGC | 236 | UCCGUG | 86 | 60 | 48 | 84 | 61 | 60 | | |
| 1228 | | CUCCUUC | | AAGGA | | | | | | | | |
| | | ACGGA | | GGCAAU | | | | | | | | |
| | | | | GC | | | | | | | | |
| UGNX | 277 | CCACGGA | 278 | UGGAAC | 89 | 69 | 35 | 84 | 61 | 63 | | |
| 1273 | | GGUUCAG | | UGAACC | | | | | | | | |
| | | UUCCA | | UCCGUG | | | | | | | | |
| | | | | G | | | | | | | | |
| UGNX | 283 | CGGAGGU | 284 | GUGUG | 90 | 64 | 37 | 77 | 55 | 48 | | |
| 1276 | | UCAGUUC | | GAACUG | | | | | | | | |
| | | CACAC | | AACCUC | | | | | | | | |
| | | | | CG | | | | | | | | |
| UGNX | 311 | CGUCCGU | 312 | GGAAU | 98 | 87 | 70 | | | | 0.240 | 2.000 |
| 1759 | | CCGUGAA | | UUCACG | | | | | | | | |
| | | AUUCC | | GACGGA | | | | | | | | |
| | | | | CG | | | | | | | | |
| UGNX | 313 | GUCCGUC | 314 | CGGAAU | 86 | 42 | 38 | | | | | |
| 1760 | | CGUGAAA | | UUCACG | | | | | | | | |
| | | UUCCG | | GACGGA | | | | | | | | |
| | | | | C | | | | | | | | |
| UGNX | 323 | GCUUUGA | 324 | UACGGG | 90 | 49 | 50 | | | | 0.870 | 1.600 |
| 1904 | | GCGGAAC | | UUCCGC | | | | | | | | |
| | | CCGUA | | UCAAAG | | | | | | | | |
| | | | | C | | | | | | | | |
| UGNX | 331 | CCAGGGU | 332 | AACCAA | 93 | 22 | −6 | | | | | |
| 1976 | | CCAGAUU | | AUCUGG | | | | | | | | |
| | | UGGUU | | ACCCUG | | | | | | | | |
| | | | | G | | | | | | | | |
| UGNX | 339 | GGUCCAG | 340 | CUGAAA | 92 | 89 | 65 | | | | 0.060 | 0.220 |
| 1980 | | AUUUGGU | | CCAAAU | | | | | | | | |
| | | UUCAG | | CUGGAC | | | | | | | | |
| | | | | C | | | | | | | | |
| UGNX | 341 | GUCCAGA | 342 | UCUGAA | 96 | 80 | 56 | | | | 0.360 | 0.073 |
| 1981 | | UUUGGUU | | ACCAAA | | | | | | | | |
| | | UCAGA | | UCUGGA | | | | | | | | |
| | | | | C | | | | | | | | |
| UGNX | 343 | UCCAGAU | 344 | UUCUGA | 87 | 50 | −18 | | | | | |
| 1982 | | UUGGUUU | | AACCAA | | | | | | | | |
| | | CAGAA | | AUCUGG | | | | | | | | |
| | | | | A | | | | | | | | |
| UGNX | 347 | CAGAUUU | 348 | CAUUCU | 98 | 88 | 58 | | | | 0.280 | 0.230 |
| 1984 | | GGUUUCA | | GAAACC | | | | | | | | |
| | | GAAUG | | AAAUCU | | | | | | | | |
| | | | | G | | | | | | | | |
| UGNX | 349 | AGAUUUG | 350 | UCAUUC | 99 | 95 | 90 | | | | 0.028 | 0.041 |
| 1985 | | GUUUCAG | | UGAAAC | | | | | | | | |
| | | AAUGA | | CAAAUC | | | | | | | | |
| | | | | U | | | | | | | | |
| UGNX | 351 | GAUUUGG | 352 | CUCAUU | 85 | 45 | 4 | | | | | |
| 1986 | | UUUCAGA | | CUGAAA | | | | | | | | |
| | | AUGAG | | CCAAAU | | | | | | | | |
| | | | | C | | | | | | | | |
| UGNX | 353 | AUUUGGU | 354 | UCUCAU | 90 | 83 | 57 | | | | 0.500 | 0.154 |
| 1987 | | UUCAGAA | | UCUGAA | | | | | | | | |
| | | UGAGA | | ACCAAA | | | | | | | | |
| | | | | U | | | | | | | | |
| UGNX | 361 | GGUUUCA | 362 | GACCUC | 93 | 60 | 13 | | | | | |
| 1991 | | GAAUGAG | | UCAUUC | | | | | | | | |
| | | AGGUC | | UGAAAC | | | | | | | | |
| | | | | C | | | | | | | | |
| UGNX | 363 | GUUUCAG | 364 | UGACCU | 96 | 91 | 89 | | | | 0.035 | 0.100 |
| 1992 | | AAUGAGA | | CUCAUU | | | | | | | | |
| | | GGUCA | | CUGAAA | | | | | | | | |
| | | | | C | | | | | | | | |
| UGNX | 371 | CAGAAUG | 372 | GGCGUG | 88 | 63 | 51 | | | | 0.610 | 3.8 |
| 1996 | | AGAGGUC | | ACCUCU | | | | | | | | |
| | | ACGCC | | CAUUCU | | | | | | | | |
| | | | | G | | | | | | | | |
| UFNX | 375 | GAAUGAG | 376 | CUGGCG | 92 | 70 | 32 | | | | | |
| 1998 | | AGGUCAC | | UGACCU | | | | | | | | |
| | | GCCAG | | CUCAUU | | | | | | | | |
| | | | | C | | | | | | | | |
| UGNX | 377 | AAUGAGA | 378 | GCUGGC | 46 | 12 | 30 | | | | | |
| 1999 | | GGUCACG | | GUGACC | | | | | | | | |
| | | CCAGC | | UCUCAU | | | | | | | | |
| | | | | U | | | | | | | | |
| UGNX | 385 | CCUCCGA | 386 | CUUCUC | 84 | 52 | 28 | | | | | |
| 2121 | | GCCUUUG | | AAAGGC | | | | | | | | |
| | | AGAAG | | UCGGAG | | | | | | | | |
| | | | | G | | | | | | | | |
| UGNX | 393 | CGAGCCU | 394 | GAUCCU | 66 | −8 | 15 | | | | | |
| 2125 | | UUGAGAA | | UCUCAA | | | | | | | | |
| | | GGAUC | | AGGCUC | | | | | | | | |
| | | | | G | | | | | | | | |
| UGNX | 399 | GCCUUUG | 400 | AGCGAU | 94 | 80 | 59 | | | | 0.061 | 0.084 |
| 2128 | | AGAAGGA | | CCUUCU | | | | | | | | |
| | | UCGCU | | CAAAGG | | | | | | | | |
| | | | | C | | | | | | | | |
| UGNX | 401 | CCUUUGA | 402 | AAGCGA | 94 | 84 | 67 | | | | 0.170 | 0.110 |
| 2129 | | GAAGGAU | | UCCUUC | | | | | | | | |
| | | CGCUU | | UCAAAG | | | | | | | | |
| | | | | G | | | | | | | | |
| UGNX | 403 | CUUUGAG | 404 | AAAGCG | 98 | 96 | 94 | | | | 0.023 | 0.230 |
| 2130 | | AAGGAUC | | AUCCUU | | | | | | | | |
| | | GCUUU | | CUCAAA | | | | | | | | |
| | | | | G | | | | | | | | |
| UGNX | 409 | UGAGAAG | 410 | UGGAA | 98 | 92 | 37 | | | | 0.370 | 0.840 |
| 2133 | | GAUCGCU | | AGCGAU | | | | | | | | |
| | | UUCCA | | CCUUCU | | | | | | | | |
| | | | | CA | | | | | | | | |
| UGNX | 421 | GGAUCGC | 422 | GAUGCC | 96 | 89 | 45 | | | | | |
| 2139 | | UUUCCAG | | UGGAA | | | | | | | | |
| | | GCAUC | | AGCGAU | | | | | | | | |
| | | | | CC | | | | | | | | |
| UGNX | 425 | CCGGAGU | 426 | UCUGAA | 83 | 49 | 32 | | | | | |
| 2194 | | CCAGGAU | | UCCUGG | | | | | | | | |
| | | UCAGA | | ACUCCG | | | | | | | | |
| | | | | G | | | | | | | | |
| UGNX | 433 | AGUCCAG | 434 | CAGAUC | 98 | 90 | 60 | | | | 0.700 | 0.540 |
| 2198 | | GAUUCAG | | UGAAUC | | | | | | | | |
| | | AUCUG | | CUGGAC | | | | | | | | |
| | | | | U | | | | | | | | |
| UGNX | 445 | GGAUUCA | 446 | UGAAAC | 91 | 82 | 60 | | | | 0.210 | 0.370 |
| 2204 | | GAUCUGG | | CAGAUC | | | | | | | | |
| | | UUUCA | | UGAAUC | | | | | | | | |
| | | | | C | | | | | | | | |
| UGNX | 447 | GAUUCAG | 448 | CUGAAA | 95 | 85 | 61 | | | | 0.220 | 0.047 |
| 2205 | | AUCUGGU | | CCAGAU | | | | | | | | |
| | | UUCAG | | CUGAAU | | | | | | | | |
| | | | | C | | | | | | | | |
| UGNX | 449 | AUUCAGA | 450 | UCUGAA | 91 | 80 | 74 | | | | 0.100 | 0.032 |
| 2206 | | UCUGGUU | | ACCAGA | | | | | | | | |
| | | UCAGA | | UCUGAA | | | | | | | | |
| | | | | U | | | | | | | | |
| UGNX | 451 | UUCAGAU | 452 | UUCUGA | 67 | −23 | 2 | | | | | |
| 2207 | | CUGGUUU | | AACCAG | | | | | | | | |
| | | CAGAA | | AUCUGA | | | | | | | | |
| | | | | A | | | | | | | | |
| UGNX | 455 | CAGAUCU | 456 | GAUUCU | 91 | 62 | 39 | | | | | |
| 2209 | | GGUUUCA | | GAAACC | | | | | | | | |
| | | GAAUC | | AGAUCU | | | | | | | | |
| | | | | G | | | | | | | | |
| UGNX | 459 | GAUCUGG | 460 | UCGAUU | 95 | 83 | 36 | | | | | |
| 2211 | | UUUCAGA | | CUGAAA | | | | | | | | |
| | | AUCGA | | CCAGAU | | | | | | | | |
| | | | | C | | | | | | | | |
| UGNX | 461 | AUCUGGU | 462 | UUCGAU | 71 | 86 | 69 | | | | 0.180 | 0.040 |
| 2212 | | UUCAGAA | | UCUGAA | | | | | | | | |
| | | UCGAA | | ACCAGA | | | | | | | | |
| | | | | U | | | | | | | | |
| UGNX | 481 | AGGCGCA | 482 | UCUAGG | 85 | 66 | 31 | | | | | |
| 2951 | | ACCUCUC | | AGAGG | | | | | | | | |
| | | CUAGA | | UUGCGC | | | | | | | | |
| | | | | CU | | | | | | | | |
| UGNX | 507 | CGAGGAA | 508 | AGCCCG | 92 | 75 | 35 | | | | | |
| 3033 | | GAAUACC | | GUAUUC | | | | | | | | |
| | | GGGCU | | UUCCUC | | | | | | | | |
| | | | | G | | | | | | | | |
| UGNX | 511 | AGGAAGA | 512 | AGAGCC | 88 | 35 | −14 | | | | | |
| 3035 | | AUACCGG | | CGGUAU | | | | | | | | |
| | | GCUCU | | UCUUCC | | | | | | | | |
| | | | | U | | | | | | | | |

### Example 4: siRNA Modifications

siRNA UGNX-1898 (comprising sense strand sequence SEQ ID NO:41 and antisense strand sequence SEQ ID NO:42) was modified by addition of TT overhang at the 3′ end of each of the sense and antisense strands, and by incorporation of 2′O-methyl modified ribose sugars in the sense strand as shown below (bold—TT overhangs, underlined—2′O-methyl).

Sense strand (5′-3′)
(SEQ ID NO: 41)
GAGCCUGCUUUGAGCGGAATT
Antisense strand (3′-5′)
(SEQ ID NO: 42)
TTCUCGGACGAAACUCGCCUU

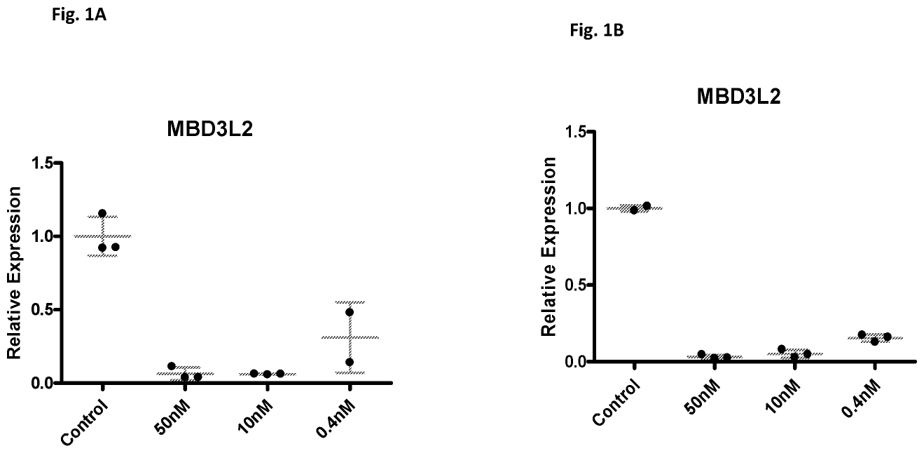

Inhibition of DUX4 by unmodified and modified siRNA was determined as explained in Example 2 by measuring the expression of MBD3L2. The data is summarized in FIG. 1A (unmodified) and FIG. 1B (modified), and demonstrates that the modified siRNA showed minimal potency loss in vitro.

### Example 5: siRNA Modifications

In this example, various chemical modifications were made to generate chemically modified siRNA compounds targeting DUX4. The chemically modified siRNAs are shown in Table 5.

TABLE 5
Chemically Modified siRNAs

| | SEQ ID | | SEQ ID | |
|---|---|---|---|---|
| siRNA name | NO | sense | NO: | antisense |
| UGNX1165a | 597 | +GrArArAmUrGrArArCmGrAmG | 598 | rUmGrUmGrGrCmUrCmUrCmGrUmUmC |
| | | rAmGrCrCrArCrA+T+T | | rAmUrUmUrCTT |
| UGNX1980a | 599 | +GrGmUrCrCrArGrAmUmUmUr | 600 | rCmUrGrArArArCrCrArArAmUrCmUrGm |
| | | GrGmUmUmUrCrArG+T+T | | GrArCrCTT |
| UGNX1981a | 601 | +GmUrCrCrArGrAmUrUmUrGrG | 602 | rUrCmUrGrArArArCrCrArArAmUrCmUr |
| | | mUrUmUrCrAmGrA+T+T | | GmGrArCTT |
| UGNX1985a | 603 | +AmGrAmUrUmUrGmGrUmUrU | 604 | rUrCrArUmUrCmUrGrArArArCrCrArArA |
| | | rCrAmGrArAmUrGrA+T+T | | mUrCmUTT |
| UGNX1987a | 605 | +AmUrUmUrGrGmUrUmUrCrA | 606 | rUrCmUrCrAmUrUrCmUrGrArArArCrCr |
| | | mGrArAmUmGrAmGrA+T+T | | ArArAmUTT |
| UGNX1992a | 607 | +GmUrUmUrCrAmGrArAmUIGr | 608 | rUmGrArCrCmUrCmUrCrArUmUrCrUmG |
| | | AmGrArmrGmUrCrA+T+T | | rArArArCTT |
| UGNX2128a | 609 | +GrCrCmUrUmUmGrAmGrArA | 610 | rAmGrCmGrAmUrCrCmUrUrCmUrCrArA |
| | | mGrGrAmUrCmGrCmU+T+T | | rAmGrGrCTT |
| UGNX2130a | 611 | +CmUrUmUmGrAmGrArAmGm | 612 | rArArAmGrCmGrAmUrCrCmUmUrCmUr |
| | | GrAmUrCmGrCmUrUmU+T+T | | CrArArAmGTT |
| UGNX2205a | 613 | +GrAmUmUrCrAmGrAmUrCmU | 614 | rCmUrGrArArArCmCrArGrAmUrCmUrGr |
| | | rGrGmUrUmUrCrAmG+T+T | | ArAmUrCTT |
| UGNX2206a | 615 | +AmUmUrCrAmGrAmUrCmUrG | 616 | rUrCmUrGrArArArCrCrAmGrAmUrCmUr |
| | | rGmUmUmUrCrAmGrA+T+T | | GrArAmUTT |
| UGNX1165b | 617 | +GrArArAmUrGrArArCmGrAmG | 618 | mUmGmUmGrGrCmUrCmUrCmGmUmU |
| | | rAmGrCrCrArCrA+T+T | | mCrAmUmUmUrC+T+T |
| UGNX1980b | 619 | +GrGmUrCrCrArGrAmUmUmUr | 620 | rCmUrGrArArArCrCrArArAmUrCmUrGm |
| | | G?GmUmUmUrCrArG+T+T | | GrArCrC+T+T |
| UGNX1981b | 621 | +GmUrCrCrArGrAmUmUmUrGr | 622 | mUrCmUrGrArArArCrCrArArAmUrCmUr |
| | | GmUmUmUrCrAmGrA+T+T | | GmGrArC+T+T |
| UGNX1985b | 623 | +AmGrAmUmUmUrGmGmUmU | 624 | mUrCrAmUmUrCmUrGrArArArCrCrArAr |
| | | mUrCrAmGrArAmUrGrA+T+T | | AmUrCmU+T+T |
| UGNX1987b | 625 | +AmUmUmUrGrGmUmUmUrCr | 626 | mUrCmUrCrAmUmUrCmUmGrArArArCr |
| | | AmGrArAmUmGrAmGrA+T+T | | CrArArAmU+T+T |
| UGNX1992b | 627 | +GmUmUmUrCrAmGrArAmUrG | 628 | mUmGrArCrCmUrCmUrCrAmUmUrCmU |
| | | rAmGrArmrGmUrCrA+T+T | | mGrArArArC+T+T |
| UGNX2128b | 629 | +GrCrCmUmUmUmGrAmGrArA | 630 | rAmGrCmGrAmUrCrCmUmUrCmUrCrAr |
| | | mGrGrAmUrCmGrCmU+T+T | | ArAmGrGrC+T+T |
| UGNX2130b | 631 | +CmUmUmUmGrAmGrArAmGm | 632 | rArArAmGrCmGrAmUrCrCmUmUrCmUr |
| | | GrAmUrCmGrCmUmUmU+T+T | | CrArArAmG+T+T |
| UGNX2205b | 633 | +GrAmUmUrCrAmGrAmUrCmU | 634 | rCmUrGrArArArCrCrAmGrAmUrCmUrGr |
| | | rGrGmUmUmUrCrAmG+T+T | | ArAmUrC+T+T |
| UGNX2206b | 635 | +AmUmUrCrAmGrAmUrCmUrG | 636 | mUrCmUrGrArArArCrCrAmGrAmUrCm |
| | | rGmUmUmUrCrAmGrA+T+T | | UrGrArAmU+T+T |
| UGNX1981c | 637 | +GmU*rCrCrArGrAmUrUmUrGr | 638 | rU*rC*mUrGrArArArCrCrArArAmUrCm |
| | | GmUrUmUrCrAmGrA+T+T | | UrGmGrArC*T*T |
| UGNX1985c | 639 | +AmGrAmUrUmUrGmGrUmUrU | 640 | rU*rC*rArUmUrCmUrGrArArArCrCrArA |
| | | rCrAmGrArAmUrGrA+T+T | | rAmUrCmU*T*T |
| UGNX1987c | 641 | +AmUrUmUrGrGmUrUmUrCrA | 642 | rU*rC*mUrCrAmUrUrCmUrGrArArArCr |
| | | mGrArAmUmGrAmGrA+T+T | | CrArArAmU*T*T |
| UGNX2128c | 643 | +GICrCmUrUmUmGrAmGrArAr | 644 | rA*mG*rCrGrArUrCrCmUrUrCmUrCrAr |
| | | GrGrAmUrCmGrCmU+T+T | | ArAmGrGrC*T*T |
| UGNX2130c | 645 | +CmUrUmUmGrArGrArAmGmG | 646 | rA*rA*rAmGrCrGrArUrCrCrUmUrCmUr |
| | | mAmUmCmGrCrUmUmU+T+T | | CrArArAmG*T*T |
| UGNX2130d | 647 | +CmUrUmUmGrArGrArAmGmG | 648 | rA*rA*rAmGrCrGrArUrCrCrUmUrCrUrCr |
| | | mAmUmCmGrCrUmUmU+T+T | | ArArAmG*T*T |
| UGNX1165c | 649 | +GrArArAmUrGrArArCmGrAmG | 650 | rU*mG*rUmGrGrCmUrCmUrCmGrUmU |
| | | rAmGrCrCrArCrA+T+T | | mCrAmUrUmUrC*T*T |
| UGNX1165d | 651 | rG*rA*rArAmUrGrArArCmGrA | 652 | +TmGrUmGrGrCmUrCmUrCmGrUmUmC |
| | | mGrAmGrCrCrArCrA*T*T | | rAmUrUmUrC+T+T |
| UGNX488c | 653 | +GrArArCmGrGrAmGmGmGrAr | 654 | mU*rC*mUrGmUrCrUmUmUrCrCrCmUr |
| | | ArAmGrArCrAmGrA+T+T | | CrCmGrUmUrC*T*T |
| UGNX488d | 655 | rG*rA*rArCmGrGrAmGmGmGr | 656 | +TrCmUrGmUrCrUmUmUrCrCrCmUrCrC |
| | | ArArAmGrArCrAmGrA*T*T | | mGrUmUrC+T+T |
| UGNX1097c | 657 | +AmUrCrCrAmUrCmGmUrCrAr | 658 | mU*rG*rAmUrGrGmCrCmUrGrArCmGr |
| | | GrGmCrCrAmUrCrA+T+T | | AmUrGrGrAmU*T*T |
| UGNX1097d | 659 | rA*mU*rCrCrAmUrCmGmUrCrA | 660 | +TrG*rAmUrGrGmCrCmUrGrArCmGrAm |
| | | rGrGmCrCrAmUrCrA*T*T | | UIGrGrAmU+T+T |
| UGNX1981d | 661 | +GmU*rCrCrAmGrAmUrUmUm | 662 | /5Phos/rU*rC*mUrGrAmArArCrCrAmAr |
| | | GIGmUrUmUrCrAmGrA+T+T | | AmUrCmUrGmGrArC*T*T |
| UGNX1981e | 663 | +GmU*rCrCrAmGrAmUrUmUm | 664 | /5Phos/rU*rC*mUrGrAmArArCrCrAmAr |
| | | GrGmUrUmUrCrAmGrA+T+T | | AmUrCmUrGmGrArC+T+T |
| UGNX1981f | 665 | +GmU*rCrCrAmGrAmUrUmUm | 666 | /5Phos/rUrC*mU*rGrAmArArCrCrAmAr |
| | | GrGmUrUmUrCrAmGrA+T+T | | AmUrCmUrGmGrArC+T+T |
| UGNX1985d | 667 | +AmGrAmUrUmUrGmGrUmUrU | 668 | /5Phos/rU*rC*rArUmUrCmUrGmArAmAr |
| | | rCrAmGrArAmUrGrA+T+T | | CrCrAmArAmUrCmU*T*T |
| UGNX1985e | 669 | +AmGrAmUrUmUrGmGrUmUrU | 670 | /5Phos/rU*rC*rArUmUrCmUrGmArAmAr |
| | | rCrAmGrArAmUrGrA+T+T | | CrCrAmArAmUrCmU+T+T |
| UGNX1987d | 671 | +AmUrUmUrGrGmUrUmUmCrA | 672 | /5Phos/rU*rC*mUrCrAmUrUrCmUrGrAm |
| | | mGmArAmUmGrAmGrA+T+T | | ArArCmCrAmArAmU*T*T |
| UGNX1987e | 673 | +AmUrUmUrGrGmUrUmUmCrA | 674 | /5Phos/rUrC*mUrCrAmUrUrCmUrGrAm |
| | | mGmArAmUmGrAmGrA+T+T | | ArArCmCrAmArAmU+T+T |
| UGNX2128d | 675 | +GrCrCmUrUmUmGrAmGrArAr | 676 | /5Phos/rA*mG*rCrGrAmUrCrCmUrUrCm |
| | | GrGrAmUrCmGrCmU+T+T | | UrCrAmArAmGrGrC*T*T |
| UGNX2128e | 677 | +GrCrCmUrUmUmGrAmGrArAr | 678 | /5Phos/rA*mG*rCrGrAmUrCrCmUrUrCm |
| | | GrGrAmUrCmGrCmU+T+T | | UrCrAmArAmGrGrC+T+T |
| UGNX2130e | 679 | +CmUrUmUmGrAmGrArAmGm | 680 | /5Phos/rA*mA*rAmGrCmGrAmUrCrCrU |
| | | GrAmUrCmGrCmUrUmU+T+T | | mUrCmUrCrAmArAmG*T*T |
| UGNX2130f | 681 | +CmUrUmUmGrAmGrArAmGm | 682 | /5Phos/rA*mA*rAmGrCmGrAmUrCrCrU |
| | | GrAmUrCmGrCmUrUmU+T+T | | mUrCmUrCrAmArAmG+T+T |
| UGNX1985- | 683 | 52FA*mG*I2FAmUI2FUmUI2FG | 684 | mU*I2FC*mAI2FUmUI2FCmUI2FGmAI2 |
| Full | | mGI2FUmUI2FUmCI2FAmGI2F | | FAmAI2FCmCI2FAmAI2FAmUI2FCmU |
| | | AmAI2FUmGI2FA*+T*+T | | *T*T |
| UGNX2130- | 685 | 52FC*mU*I2FUmUI2FGmAI2FG | 686 | mA*I2FA*mAI2FGmCI2FGmAI2FUmCI2 |
| Full | | mAI2FAmGI2FGmAI2FUmCI2F | | FCmUI2FUmCI2FUmCI2FAmAI2FAmG |
| | | GmCI2FUmUI2FU+T+T | | *T*T |
| UGNX2206- | 687 | 52FA*mU*I2FUmCI2FAmGI2FA | 688 | mU*I2FC*mUI2FGmAI2FAmAI2FCmCI2 |
| Full | | mUI2FCmUI2FGmGI2FUmUI2F | | FAmGI2FAmUI2FCmUI2FGmAI2FAmU |
| | | UmCI2FAmGI2FA*+T*+T | | *T*T |

Modifications in Table 5:
52FA =5′ 2′-Fluoro A (a 2-Fluoro A modification at the 5′ end)
52FC = 5′ 2′-Fluoro C (a 2-Fluoro C modification at the 5′ end)
I2FA = Int 2′-Fluoro A (an internal 2-Fluoro A modification)
I2FC = Int 2′-Fluoro C (an internal 2-Fluoro C modification)
I2FG = Int 2′-Fluoro G (an internal 2-Fluoro G modification)
I2FU = Int 2′-Fluoro U (an internal 2-Fluoro U modification)
m = 2′ O-Methyl RNA base
* = Phosphorothioated DNA (phosphorothioate bonds in these positions)
r = RNA base
+ = Locked Nucleic Acid (LNA)

Following synthesis, the chemically modified compounds shown in Table 5 were evaluated for potency and stability. Results are shown in Table 6.

MB200 and 54-2 potency assays were performed as described in Examples 2-3, while potency in a cultured human hepatocyte carcinoma cell line (HepG2) was measured as follows. Briefly, a human hepatocyte carcinoma cell line HepG2 was cryo-recovered and plated. Cells were seeded at 1×10⁵ cells/well in 90 ul EMEM+10% FBS in 96-well culture plates. Chemically modified siRNA sequences were then transfected using Lipofectamine RNAiMAX (Thermo-Fisher Scientific) at varying amounts. After 24 hours post-transfection, the cells were lysed in lysis buffer and harvested for subsequent Quantigene Singleplex Gene Expression analysis (Thermo-Fisher Scientific). HepG2 potency values were measured as relative DUX4 gene expression normalized to negative control siRNA, with +=0.5 and ++=<0.5 as illustrated in Table 6.

Meanwhile, the stability of the chemically modified siRNAs was measured in human serum as follows. First, 1 uM of each siRNA sequence was incubated with 10% human serum for 2 hours in a heat block at 37° C. Additionally, 1 uM of each siRNA sequence was incubated without serum at the same conditions. After incubation, the siRNA/serum and siRNA alone mixtures were snap frozen on dry ice. Next, to determine stability, the Agilent Small RNA Assay was run on the samples following manufacturer's protocol for preparation of the chip. Samples were run on Agilent 2100 Bioanalyzer instrument. Stability was determined by peak presence in electropherograms and band presence in translated electrophoresis gel. Quantitation was measured by size distribution of the peak to represent percent recovery, with 50%=+, 50-75%=++, and greater than 75%=+++ as illustrated in Table 6.

TABLE 6
Potency and Stability of Chemically Modified siRNAs

| | | Potency | | |
|---|---|---|---|---|
| siRNA name | Stability | MB200* | 54-2* | Hep G2 |
| UGNX1165a | Not tested | Not active | Not active | + |
| UGNX1980a | Not tested | Not active | Not active | + |
| UGNX1981a | − | 0.02 | 0.072 | + |
| UGNX1985a | + | 0.025 | 0.018 | ++ |
| UGNX1987a | ++ | 0.024 | 0.038 | ++ |
| UGNX1992a | Not tested | 1.1 | 0.79 | ++ |
| UGNX2128a | Not tested | Not active | Not active | + |
| UGNX2130a | ++ | 3.1 | 0.83 | + |
| UGNX2205a | Not tested | Not active | Not active | − |
| UGNX2206a | − | 0.018 | 0.022 | + |
| UGNX1165b | Not tested | − | − | − |
| UGNX1980b | Not tested | 27% | − | − |
| UGNX1981b | ++ | 66% | − | − |
| UGNX1985b | | 0.27 | 0.69 | − |
| UGNX1987b | Not tested | 89% | 7.2 | − |
| UGNX1992b | Not tested | 43% | − | − |
| UGNX2128b | Not tested | 46% | − | − |
| UGNX2130b | | 52% | >10 | − |
| UGNX2205b | Not tested | | − | − |
| UGNX2206b | Not tested | 0.47 | 0.43 | − |
| UGNX1165c | ++ | − | 29% | − |
| UGNX1981c | − | 0.92 | 1.5 | + |
| UGNX1985c | + | 0.35 | 0.3 | + |
| UGNX1987c | ++ | 0.58 | 0.57 | + |
| UGNX2128c | ++ | − | − | − |
| UGNX2130c | ++ | − | − | − |
| UGNX488c | | − | 43% | − |
| UGNX1097c | | − | 33% | − |
| UGNX1165d | + | − | 26% | − |
| UGNX1981d | − | 66% | 37% | + |
| UGNX1985d | + | 0.75 | 0.72 | + |
| UGNX1987d | ++ | 3 | 5 | + |
| UGNX2128d | ++ | 2.5 | − | + |
| UGNX2130d | ++ | 1.2 | 0.38 | − |
| UGNX488d | − | − | 28% | − |
| UGNX1097d | − | − | 25% | − |
| UGNX1981e | − | 0.94 | 1.5 | |
| UGNX1985e | + | 0.28 | 0.86 | + |
| UGNX1987e | ++ | 1.1 | 0.99 | + |
| UGNX2128e | + | − | − | + |
| UGNX2130e | ++ | − | − | + |
| UGNX1981f | ++ | 2.9 | 3.3 | + |
| UGNX2130f | ++ | − | − | + |
| UGNX1985-Full | +++ | 0.5 | 0.37 | Not tested |
| UGNX2130-Full | | 0.81 | 0.64 | Not tested |
| UGNX2206-Full | +++ | 1.2 | 1.6 | Not tested |

Nomenclature in Table 6:
*Where a % value is provided, the % value is the percent inhibition at 10 nM of chemically modified siRNA; where a numerical value is provided, the numerical value represents the IC50 of the chemically modified siRNA.
(−) = Not active when used in reference to potency

1. A double-stranded small interfering RNA (siRNA) comprising a sense strand and an antisense strand,
wherein the antisense strand of the double-stranded siRNA comprises a nucleobase sequence of at least 12 contiguous nucleotides of a sequence selected from the group consisting of SEQ ID NOs: 350, 404, 450, 348, 446, 462, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, −74, 76, −78, 80, 82, 84, 86, 88, 90, 92, −94, 96, 98, 100, 102, 104, 106, 108, 110, 112, −114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, −312, 314, 316, 318, 320, 322, 324, 326, 328, 330, 332, 334, 336, 338, 340, 342, 344, 346, 352, 354, 356, 358, 360, 362, 364, 366, 368, 370, 372, 374, 376, 378, 380, 382, 384, 386, 388, 390, 392, 394, 396, 398, 400, 402, 406, 408, 410, 412, 414, 416, 418, 420, 422, 424, 426, 428, 430, 432, 434, 436, 438, 440, 442, 444, 448, 452, 454, 456, 458, 460, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, and 592,
and wherein the double-stranded siRNA comprises at least one modified nucleoside.

2. The double-stranded siRNA of claim 1, wherein the antisense strand of the double-stranded siRNA comprises a nucleobase sequence of at least 12 contiguous nucleotides of a sequence selected from the group consisting of SEQ ID NOs: 350, 404, 450, 348, 446, 462, 216, 218, 220, 312, 324, 340, 342, 352, 354, 364, 372, 376, 400, 402, 410, 434, 448, and 564.

3. (canceled)

4. (canceled)

5. (canceled)

6. The double-stranded siRNA of claim 1, wherein at least one nucleoside of the sense strand of the double-stranded siRNA comprises a modified sugar.

7. The double-stranded siRNA of claim 1, wherein each nucleoside of the sense strand of the double-stranded siRNA comprises a modified sugar.

8. The double-stranded siRNA of claim 6, wherein the modified sugar is selected from a 2′-OMe modified sugar and a 2′-F modified sugar.

9. The double-stranded siRNA of claim 1, wherein the antisense strand comprises a TT overhang at the 3′ end.

10. The double-stranded siRNA of claim 1, wherein the sense strand comprises a TT overhang at the 3′ end.

11. The double-stranded siRNA of claim 1, wherein the sense strand of the double-stranded siRNA comprises at least one modified internucleoside linkage.

12. The double-stranded siRNA of claim 11, wherein the sense strand of the double-stranded siRNA comprises at least five modified internucleoside linkages.

13. The double-stranded siRNA of claim 11, wherein each internucleoside linkage is a phosphorothioate internucleoside linkage.

14. The double-stranded siRNA of claim 1, wherein the siRNA is conjugated to a lipophilic molecule, an antibody, an aptamer, a ligand, a peptide, or a polymer.

15. The double-stranded siRNA of claim 14, wherein the lipophilic molecule is a long chain fatty acid (LCFA).

16. The double-stranded siRNA of claim 14, wherein the antibody is an anti-transferrin receptor antibody.

17. A pharmaceutical composition comprising the double-stranded siRNA of claim 1 and a pharmaceutically acceptable carrier.

18. (canceled)

19. (canceled)

20. (canceled)

21. (canceled)

22. (canceled)

23. A method for ameliorating, preventing, delaying onset of, or treating a disease or disorder associated with aberrant expression of DUX4 in a subject in need thereof, the method comprising administering to the subject the pharmaceutical composition of claim 17.

24. The method of claim 23, wherein the disease or disorder is FSHD.

25. The method of claim 24, wherein the FSHD is selected from the group consisting of FSHD1 and FSHD2.

26. (canceled)

27. (canceled)

28. (canceled)

29. (canceled)

30. The method of claim 23, wherein the administration is intravenous, subcutaneous, pulmonary, intramuscular, intraperitoneal, dermal, oral, nasal, or via inhalation.

31. (canceled)

32. (canceled)

33. (canceled)

34. (canceled)

35. (canceled)

36. (canceled)

37. A double-stranded siRNA comprising a sense strand and an antisense strand, wherein the antisense strand comprises at least 8 contiguous nucleotides of a sequence selected from the group consisting of SEQ ID NOs 604, 612, 616, 632, 636, 646, 648, 680, 682, 686, 688, 598, 600, 602, 606, 608, 610, 614, 618, 620, 622, 624, 626, 628, 630, 634, 638, 640, 642, 644, 650, 652, 654, 656, 658, 660, 662, 664, 666, 668, 670, 672, 674, 676, 678, and 684.

38.-56. (canceled)

57. A method of inhibiting expression of DUX4 in a cell, comprising contacting the cell with the double-stranded siRNA of claim 1.


SEQUENCE LISTING (692 sequences)

SEQ ID NO: 1 - RNA, 19 nt, Artificial Sequence
  Synthetic Oligo
ggcagagauggagagagga

SEQ ID NO: 2 - RNA, 19 nt, Artificial Sequence
  Synthetic Oligo
uccucucuccaucucugcc

SEQ ID NO: 3 - RNA, 19 nt, Artificial Sequence
  Synthetic Oligo
agggaggaacggagggaaa

SEQ ID NO: 4 - RNA, 19 nt, Artificial Sequence
  Synthetic Oligo
uuucccuccguuccucccu

SEQ ID NO: 5 - RNA, 19 nt, Artificial Sequence
  Synthetic Oligo
gaggaaggcagggaggaaa

SEQ ID NO: 6 - RNA, 19 nt, Artificial Sequence
  Synthetic Oligo
uuuccucccugccuuccuc

SEQ ID NO: 7 - RNA, 19 nt, Artificial Sequence
  Synthetic Oligo
cgguuuccuccgggacaaa

SEQ ID NO: 8 - RNA, 19 nt, Artificial Sequence
  Synthetic Oligo
uuugucccggaggaaaccg

SEQ ID NO: 9 - RNA, 19 nt, Artificial Sequence
  Synthetic Oligo
cgguucacagaccgcacau

SEQ ID NO: 10 - RNA, 19 nt, Artificial Sequence
  Synthetic Oligo
augugcggucugugaaccg

SEQ ID NO: 11 - RNA, 19 nt, Artificial Sequence
  Synthetic Oligo
gacgacggaggcgugauuu

SEQ ID NO: 12 - RNA, 19 nt, Artificial Sequence
  Synthetic Oligo
aaaucacgccuccgucguc

SEQ ID NO: 13 - RNA, 19 nt, Artificial Sequence
  Synthetic Oligo
gccuguugcucacgucucu

SEQ ID NO: 14 - RNA, 19 nt, Artificial Sequence
  Synthetic Oligo
agagacgugagcaacaggc

SEQ ID NO: 15 - RNA, 19 nt, Artificial Sequence
  Synthetic Oligo
cuggccaugccgacuguuu

SEQ ID NO: 16 - RNA, 19 nt, Artificial Sequence
  Synthetic Oligo
aaacagucggcauggccag

SEQ ID NO: 17 - RNA, 19 nt, Artificial Sequence
  Synthetic Oligo
ccggaaacaugcagggaag

SEQ ID NO: 18 - RNA, 19 nt, Artificial Sequence
  Synthetic Oligo
cuucccugcauguuuccgg

SEQ ID NO: 19 - RNA, 19 nt, Artificial Sequence
  Synthetic Oligo
uucgcucuccuugccaggu

SEQ ID NO: 20 - RNA, 19 nt, Artificial Sequence
  Synthetic Oligo
accuggcaaggagagcgaa

SEQ ID NO: 21 - RNA, 19 nt, Artificial Sequence
  Synthetic Oligo
ggaauccaucgucaggcca

SEQ ID NO: 22 - RNA, 19 nt, Artificial Sequence
  Synthetic Oligo
uggccugacgauggauucc

SEQ ID NO: 23 - RNA, 19 nt, Artificial Sequence
  Synthetic Oligo
gucucgcucuggucuucua

SEQ ID NO: 24 - RNA, 19 nt, Artificial Sequence
  Synthetic Oligo
uagaagaccagagcgagac

SEQ ID NO: 25 - RNA, 19 nt, Artificial Sequence
  Synthetic Oligo
cacaggcauugccuccuuc

SEQ ID NO: 26 - RNA, 19 nt, Artificial Sequence
  Synthetic Oligo
gaaggaggcaaugccugug

SEQ ID NO: 27 - RNA, 19 nt, Artificial Sequence
  Synthetic Oligo
gccuggcacacucaagacu

SEQ ID NO: 28 - RNA, 19 nt, Artificial Sequence
  Synthetic Oligo
agucuugagugugccaggc

SEQ ID NO: 29 - RNA, 19 nt, Artificial Sequence
  Synthetic Oligo
aggcugguuucucccugcu

SEQ ID NO: 30 - RNA, 19 nt, Artificial Sequence
  Synthetic Oligo
agcagggagaaaccagccu

SEQ ID NO: 31 - RNA, 19 nt, Artificial Sequence
  Synthetic Oligo
cuuccucuucgucucuccg

SEQ ID NO: 32 - RNA, 19 nt, Artificial Sequence
  Synthetic Oligo
cggagagacgaagaggaag

SEQ ID NO: 33 - RNA, 19 nt, Artificial Sequence
  Synthetic Oligo
cucaccgccauucaugaag

SEQ ID NO: 34 - RNA, 19 nt, Artificial Sequence
  Synthetic Oligo
cuucaugaauggcggugag

SEQ ID NO: 35 - RNA, 19 nt, Artificial Sequence
  Synthetic Oligo
cugccugugggccuuuaca

SEQ ID NO: 36 - RNA, 19 nt, Artificial Sequence
  Synthetic Oligo
uguaaaggcccacaggcag

SEQ ID NO: 37 - RNA, 19 nt, Artificial Sequence
  Synthetic Oligo
gcguccguccgugaaauuc

SEQ ID NO: 38 - RNA, 19 nt, Artificial Sequence
  Synthetic Oligo
gaauuucacggacggacgc

SEQ ID NO: 39 - RNA, 19 nt, Artificial Sequence
  Synthetic Oligo
cgacggagacucguuugga

SEQ ID NO: 40 - RNA, 19 nt, Artificial Sequence
  Synthetic Oligo
uccaaacgagucuccgucg

SEQ ID NO: 41 - RNA, 19 nt, Artificial Sequence
  Synthetic Oligo
gagccugcuuugagcggaa

SEQ ID NO: 42 - RNA, 19 nt, Artificial Sequence
  Synthetic Oligo
uuccgcucaaagcaggcuc

SEQ ID NO: 43 - RNA, 19 nt, Artificial Sequence
  Synthetic Oligo
gagcccaggguccagauuu

SEQ ID NO: 44 - RNA, 19 nt, Artificial Sequence
  Synthetic Oligo
aaaucuggacccugggcuc

SEQ ID NO: 45 - RNA, 19 nt, Artificial Sequence
  Synthetic Oligo
cuccuccgagccuuugaga

SEQ ID NO: 46 - RNA, 19 nt, Artificial Sequence
  Synthetic Oligo
ucucaaaggcucggaggag

SEQ ID NO: 47 - RNA, 19 nt, Artificial Sequence
  Synthetic Oligo
ucccggaguccaggauuca

SEQ ID NO: 48 - RNA, 19 nt, Artificial Sequence
  Synthetic Oligo
ugaauccuggacuccggga

SEQ ID NO: 49 - RNA, 19 nt, Artificial Sequence
  Synthetic Oligo
ugcugcuggaugagcuccu

SEQ ID NO: 50 - RNA, 19 nt, Artificial Sequence
  Synthetic Oligo
aggagcucauccagcagca

SEQ ID NO: 51 - RNA, 19 nt, Artificial Sequence
  Synthetic Oligo
ggcgcaaccucuccuagaa

SEQ ID NO: 52 - RNA, 19 nt, Artificial Sequence
  Synthetic Oligo
uucuaggagagguugcgcc

SEQ ID NO: 53 - RNA, 19 nt, Artificial Sequence
  Synthetic Oligo
cucagcgaggaagaauacc

SEQ ID NO: 54 - RNA, 19 nt, Artificial Sequence
  Synthetic Oligo
gguauucuuccucgcugag

SEQ ID NO: 55 - RNA, 19 nt, Artificial Sequence
  Synthetic Oligo
cugggauuccugccuucua

SEQ ID NO: 56 - RNA, 19 nt, Artificial Sequence
  Synthetic Oligo
uagaaggcaggaaucccag

SEQ ID NO: 57 - RNA, 19 nt, Artificial Sequence
  Synthetic Oligo
gcggagaacugccauucuu

SEQ ID NO: 58 - RNA, 19 nt, Artificial Sequence
  Synthetic Oligo
aagaauggcaguucuccgc

SEQ ID NO: 59 - RNA, 19 nt, Artificial Sequence
  Synthetic Oligo
gcagagauggagagaggaa

SEQ ID NO: 60 - RNA, 19 nt, Artificial Sequence
  Synthetic Oligo
uuccucucuccaucucugc

SEQ ID NO: 61 - RNA, 19 nt, Artificial Sequence
  Synthetic Oligo
cagagauggagagaggaac

SEQ ID NO: 62 - RNA, 19 nt, Artificial Sequence
  Synthetic Oligo
guuccucucuccaucucug

SEQ ID NO: 63 - RNA, 19 nt, Artificial Sequence
  Synthetic Oligo
agagauggagagaggaacg

SEQ ID NO: 64 - RNA, 19 nt, Artificial Sequence
  Synthetic Oligo
cguuccucucuccaucucu

SEQ ID NO: 65 - RNA, 19 nt, Artificial Sequence
  Synthetic Oligo
gagauggagagaggaacgg

SEQ ID NO: 66 - RNA, 19 nt, Artificial Sequence
  Synthetic Oligo
ccguuccucucuccaucuc

SEQ ID NO: 67 - RNA, 19 nt, Artificial Sequence
  Synthetic Oligo
agauggagagaggaacggg

SEQ ID NO: 68 - RNA, 19 nt, Artificial Sequence
  Synthetic Oligo
cccguuccucucuccaucu

SEQ ID NO: 69 - RNA, 19 nt, Artificial Sequence
  Synthetic Oligo
gauggagagaggaacggga

SEQ ID NO: 70 - RNA, 19 nt, Artificial Sequence
  Synthetic Oligo
ucccguuccucucuccauc

SEQ ID NO: 71 - RNA, 19 nt, Artificial Sequence
  Synthetic Oligo
auggagagaggaacgggag

SEQ ID NO: 72 - RNA, 19 nt, Artificial Sequence
  Synthetic Oligo
cucccguuccucucuccau

SEQ ID NO: 73 - RNA, 19 nt, Artificial Sequence
  Synthetic Oligo
uggagagaggaacgggaga

SEQ ID NO: 74 - RNA, 19 nt, Artificial Sequence
  Synthetic Oligo
ucucccguuccucucucca

SEQ ID NO: 75 - RNA, 19 nt, Artificial Sequence
  Synthetic Oligo
agagaggaacgggagaccu

SEQ ID NO: 76 - RNA, 19 nt, Artificial Sequence
  Synthetic Oligo
aggucucccguuccucucu

SEQ ID NO: 77 - RNA, 19 nt, Artificial Sequence
  Synthetic Oligo
gagaggaacgggagaccua

SEQ ID NO: 78 - RNA, 19 nt, Artificial Sequence
  Synthetic Oligo
uaggucucccguuccucuc

SEQ ID NO: 79 - RNA, 19 nt, Artificial Sequence
  Synthetic Oligo
agaggaacgggagaccuag

SEQ ID NO: 80 - RNA, 19 nt, Artificial Sequence
  Synthetic Oligo
cuaggucucccguuccucu

SEQ ID NO: 81 - RNA, 19 nt, Artificial Sequence
  Synthetic Oligo
gaggaacgggagaccuaga

SEQ ID NO: 82 - RNA, 19 nt, Artificial Sequence
  Synthetic Oligo
ucuaggucucccguuccuc

SEQ ID NO: 83 - RNA, 19 nt, Artificial Sequence
  Synthetic Oligo
aggaacgggagaccuagag

SEQ ID NO: 84 - RNA, 19 nt, Artificial Sequence
  Synthetic Oligo
cucuaggucucccguuccu

SEQ ID NO: 85 - RNA, 19 nt, Artificial Sequence
  Synthetic Oligo
ggaggaacggagggaaaga

SEQ ID NO: 86 - RNA, 19 nt, Artificial Sequence
  Synthetic Oligo
ucuuucccuccguuccucc

SEQ ID NO: 87 - RNA, 19 nt, Artificial Sequence
  Synthetic Oligo
gaggaacggagggaaagac

SEQ ID NO: 88 - RNA, 19 nt, Artificial Sequence
  Synthetic Oligo
gucuuucccuccguuccuc

SEQ ID NO: 89 - RNA, 19 nt, Artificial Sequence
  Synthetic Oligo
aggaacggagggaaagaca

SEQ ID NO: 90 - RNA, 19 nt, Artificial Sequence
  Synthetic Oligo
ugucuuucccuccguuccu

SEQ ID NO: 91 - RNA, 19 nt, Artificial Sequence
  Synthetic Oligo
ggaacggagggaaagacag

SEQ ID NO: 92 - RNA, 19 nt, Artificial Sequence
  Synthetic Oligo
cugucuuucccuccguucc

SEQ ID NO: 93 - RNA, 19 nt, Artificial Sequence
  Synthetic Oligo
gaacggagggaaagacaga

SEQ ID NO: 94 - RNA, 19 nt, Artificial Sequence
  Synthetic Oligo
ucugucuuucccuccguuc

SEQ ID NO: 95 - RNA, 19 nt, Artificial Sequence
  Synthetic Oligo
aacggagggaaagacagag

SEQ ID NO: 96 - RNA, 19 nt, Artificial Sequence
  Synthetic Oligo
cucugucuuucccuccguu

SEQ ID NO: 97 - RNA, 19 nt, Artificial Sequence
  Synthetic Oligo
acggagggaaagacagagc

SEQ ID NO: 98 - RNA, 19 nt, Artificial Sequence
  Synthetic Oligo
gcucugucuuucccuccgu

SEQ ID NO: 99 - RNA, 19 nt, Artificial Sequence
  Synthetic Oligo
ggagggaaagacagagcga

SEQ ID NO: 100 - RNA, 19 nt, Artificial Sequence
  Synthetic Oligo
ucgcucugucuuucccucc

SEQ ID NO: 101 - RNA, 19 nt, Artificial Sequence
  Synthetic Oligo
gagggaaagacagagcgac

SEQ ID NO: 102 - RNA, 19 nt, Artificial Sequence
  Synthetic Oligo
gucgcucugucuuucccuc

SEQ ID NO: 103 - RNA, 19 nt, Artificial Sequence
  Synthetic Oligo
agggaaagacagagcgacg

SEQ ID NO: 104 - RNA, 19 nt, Artificial Sequence
  Synthetic Oligo
cgucgcucugucuuucccu

SEQ ID NO: 105 - RNA, 19 nt, Artificial Sequence
  Synthetic Oligo
ggaaagacagagcgacgca

SEQ ID NO: 106 - RNA, 19 nt, Artificial Sequence
  Synthetic Oligo
ugcgucgcucugucuuucc

SEQ ID NO: 107 - RNA, 19 nt, Artificial Sequence
  Synthetic Oligo
gaaagacagagcgacgcag

SEQ ID NO: 108 - RNA, 19 nt, Artificial Sequence
  Synthetic Oligo
cugcgucgcucugucuuuc

SEQ ID NO: 109 - RNA, 19 nt, Artificial Sequence
  Synthetic Oligo
aaagacagagcgacgcagg

SEQ ID NO: 110 - RNA, 19 nt, Artificial Sequence
  Synthetic Oligo
ccugcgucgcucugucuuu

SEQ ID NO: 111 - RNA, 19 nt, Artificial Sequence
  Synthetic Oligo
gguucacagaccgcacauc

SEQ ID NO: 112 - RNA, 19 nt, Artificial Sequence
  Synthetic Oligo
gaugugcggucugugaacc

SEQ ID NO: 113 - RNA, 19 nt, Artificial Sequence
  Synthetic Oligo
guucacagaccgcacaucc

SEQ ID NO: 114 - RNA, 19 nt, Artificial Sequence
  Synthetic Oligo
ggaugugcggucugugaac

SEQ ID NO: 115 - RNA, 19 nt, Artificial Sequence
  Synthetic Oligo
uucacagaccgcacauccc

SEQ ID NO: 116 - RNA, 19 nt, Artificial Sequence
  Synthetic Oligo
gggaugugcggucugugaa

SEQ ID NO: 117 - RNA, 19 nt, Artificial Sequence
  Synthetic Oligo
ccuguugcucacgucucuc

SEQ ID NO: 118 - RNA, 19 nt, Artificial Sequence
  Synthetic Oligo
gagagacgugagcaacagg

SEQ ID NO: 119 - RNA, 19 nt, Artificial Sequence
  Synthetic Oligo
cuguugcucacgucucucc

SEQ ID NO: 120 - RNA, 19 nt, Artificial Sequence
  Synthetic Oligo
ggagagacgugagcaacag

SEQ ID NO: 121 - RNA, 19 nt, Artificial Sequence
  Synthetic Oligo
uguugcucacgucucuccg

SEQ ID NO: 122 - RNA, 19 nt, Artificial Sequence
  Synthetic Oligo
cggagagacgugagcaaca

SEQ ID NO: 123 - RNA, 19 nt, Artificial Sequence
  Synthetic Oligo
ccaugccgacuguuugcuc

SEQ ID NO: 124 - RNA, 19 nt, Artificial Sequence
  Synthetic Oligo
gagcaaacagucggcaugg

SEQ ID NO: 125 - RNA, 19 nt, Artificial Sequence
  Synthetic Oligo
caugccgacuguuugcucc

SEQ ID NO: 126 - RNA, 19 nt, Artificial Sequence
  Synthetic Oligo
ggagcaaacagucggcaug

SEQ ID NO: 127 - RNA, 19 nt, Artificial Sequence
  Synthetic Oligo
augccgacuguuugcuccc

SEQ ID NO: 128 - RNA, 19 nt, Artificial Sequence
  Synthetic Oligo
gggagcaaacagucggcau

SEQ ID NO: 129 - RNA, 19 nt, Artificial Sequence
  Synthetic Oligo
uguuugcucccggagcucu

SEQ ID NO: 130 - RNA, 19 nt, Artificial Sequence
  Synthetic Oligo
agagcuccgggagcaaaca

SEQ ID NO: 131 - RNA, 19 nt, Artificial Sequence
  Synthetic Oligo
cccggaaacaugcagggaa

SEQ ID NO: 132 - RNA, 19 nt, Artificial Sequence
  Synthetic Oligo
uucccugcauguuuccggg

SEQ ID NO: 133 - RNA, 19 nt, Artificial Sequence
  Synthetic Oligo
cggaaacaugcagggaagg

SEQ ID NO: 134 - RNA, 19 nt, Artificial Sequence
  Synthetic Oligo
ccuucccugcauguuuccg

SEQ ID NO: 135 - RNA, 19 nt, Artificial Sequence
  Synthetic Oligo
ggaaacaugcagggaaggg

SEQ ID NO: 136 - RNA, 19 nt, Artificial Sequence
  Synthetic Oligo
cccuucccugcauguuucc

SEQ ID NO: 137 - RNA, 19 nt, Artificial Sequence
  Synthetic Oligo
gaaacaugcagggaagggu

SEQ ID NO: 138 - RNA, 19 nt, Artificial Sequence
  Synthetic Oligo
acccuucccugcauguuuc

SEQ ID NO: 139 - RNA, 19 nt, Artificial Sequence
  Synthetic Oligo
aaacaugcagggaagggug

SEQ ID NO: 140 - RNA, 19 nt, Artificial Sequence
  Synthetic Oligo
cacccuucccugcauguuu

SEQ ID NO: 141 - RNA, 19 nt, Artificial Sequence
  Synthetic Oligo
aacaugcagggaagggugc

SEQ ID NO: 142 - RNA, 19 nt, Artificial Sequence
  Synthetic Oligo
gcacccuucccugcauguu

SEQ ID NO: 143 - RNA, 19 nt, Artificial Sequence
  Synthetic Oligo
caugcagggaagggugcaa

SEQ ID NO: 144 - RNA, 19 nt, Artificial Sequence
  Synthetic Oligo
uugcacccuucccugcaug

SEQ ID NO: 145 - RNA, 19 nt, Artificial Sequence
  Synthetic Oligo
augcagggaagggugcaag

SEQ ID NO: 146 - RNA, 19 nt, Artificial Sequence
  Synthetic Oligo
cuugcacccuucccugcau

SEQ ID NO: 147 - RNA, 19 nt, Artificial Sequence
  Synthetic Oligo
ucgcucuccuugccagguu

SEQ ID NO: 148 - RNA, 19 nt, Artificial Sequence
  Synthetic Oligo
aaccuggcaaggagagcga

SEQ ID NO: 149 - RNA, 19 nt, Artificial Sequence
  Synthetic Oligo
cucuccuugccagguucca

SEQ ID NO: 150 - RNA, 19 nt, Artificial Sequence
  Synthetic Oligo
uggaaccuggcaaggagag

SEQ ID NO: 151 - RNA, 19 nt, Artificial Sequence
  Synthetic Oligo
ucuccuugccagguuccaa

SEQ ID NO: 152 - RNA, 19 nt, Artificial Sequence
  Synthetic Oligo
uuggaaccuggcaaggaga

SEQ ID NO: 153 - RNA, 19 nt, Artificial Sequence
  Synthetic Oligo
cuccuugccagguuccaaa

SEQ ID NO: 154 - RNA, 19 nt, Artificial Sequence
  Synthetic Oligo
uuuggaaccuggcaaggag

SEQ ID NO: 155 - RNA, 19 nt, Artificial Sequence
  Synthetic Oligo
uccuugccagguuccaaac

SEQ ID NO: 156 - RNA, 19 nt, Artificial Sequence
  Synthetic Oligo
guuuggaaccuggcaagga

SEQ ID NO: 157 - RNA, 19 nt, Artificial Sequence
  Synthetic Oligo
ccuugccagguuccaaacc

SEQ ID NO: 158 - RNA, 19 nt, Artificial Sequence
  Synthetic Oligo
gguuuggaaccuggcaagg

SEQ ID NO: 159 - RNA, 19 nt, Artificial Sequence
  Synthetic Oligo
cuugccagguuccaaaccg

SEQ ID NO: 160 - RNA, 19 nt, Artificial Sequence
  Synthetic Oligo
cgguuuggaaccuggcaag

SEQ ID NO: 161 - RNA, 19 nt, Artificial Sequence
  Synthetic Oligo
uugccagguuccaaaccgg

SEQ ID NO: 162 - RNA, 19 nt, Artificial Sequence
  Synthetic Oligo
ccgguuuggaaccuggcaa

SEQ ID NO: 163 - RNA, 19 nt, Artificial Sequence
  Synthetic Oligo
guuccaaaccggccacacu

SEQ ID NO: 164 - RNA, 19 nt, Artificial Sequence
  Synthetic Oligo
aguguggccgguuuggaac

SEQ ID NO: 165 - RNA, 19 nt, Artificial Sequence
  Synthetic Oligo
uuccaaaccggccacacug

SEQ ID NO: 166 - RNA, 19 nt, Artificial Sequence
  Synthetic Oligo
caguguggccgguuuggaa

SEQ ID NO: 167 - RNA, 19 nt, Artificial Sequence
  Synthetic Oligo
gaauccaucgucaggccau

SEQ ID NO: 168 - RNA, 19 nt, Artificial Sequence
  Synthetic Oligo
auggccugacgauggauuc

SEQ ID NO: 169 - RNA, 19 nt, Artificial Sequence
  Synthetic Oligo
aauccaucgucaggccauc

SEQ ID NO: 170 - RNA, 19 nt, Artificial Sequence
  Synthetic Oligo
gauggccugacgauggauu

SEQ ID NO: 171 - RNA, 19 nt, Artificial Sequence
  Synthetic Oligo
auccaucgucaggccauca

SEQ ID NO: 172 - RNA, 19 nt, Artificial Sequence
  Synthetic Oligo
ugauggccugacgauggau

SEQ ID NO: 173 - RNA, 19 nt, Artificial Sequence
  Synthetic Oligo
uccaucgucaggccaucac

SEQ ID NO: 174 - RNA, 19 nt, Artificial Sequence
  Synthetic Oligo
gugauggccugacgaugga

SEQ ID NO: 175 - RNA, 19 nt, Artificial Sequence
  Synthetic Oligo
ucucgcucuggucuucuac

SEQ ID NO: 176 - RNA, 19 nt, Artificial Sequence
  Synthetic Oligo
guagaagaccagagcgaga

SEQ ID NO: 177 - RNA, 19 nt, Artificial Sequence
  Synthetic Oligo
cucgcucuggucuucuacg

SEQ ID NO: 178 - RNA, 19 nt, Artificial Sequence
  Synthetic Oligo
cguagaagaccagagcgag

SEQ ID NO: 179 - RNA, 19 nt, Artificial Sequence
  Synthetic Oligo
ucgcucuggucuucuacgu

SEQ ID NO: 180 - RNA, 19 nt, Artificial Sequence
  Synthetic Oligo
acguagaagaccagagcga

SEQ ID NO: 181 - RNA, 19 nt, Artificial Sequence
  Synthetic Oligo
cgcucuggucuucuacgug

SEQ ID NO: 182 - RNA, 19 nt, Artificial Sequence
  Synthetic Oligo
cacguagaagaccagagcg

SEQ ID NO: 183 - RNA, 19 nt, Artificial Sequence
  Synthetic Oligo
gcucuggucuucuacgugg

SEQ ID NO: 184 - RNA, 19 nt, Artificial Sequence
  Synthetic Oligo
ccacguagaagaccagagc

SEQ ID NO: 185 - RNA, 19 nt, Artificial Sequence
  Synthetic Oligo
cucuggucuucuacgugga

SEQ ID NO: 186 - RNA, 19 nt, Artificial Sequence
  Synthetic Oligo
uccacguagaagaccagag

SEQ ID NO: 187 - RNA, 19 nt, Artificial Sequence
  Synthetic Oligo
ucuggucuucuacguggaa

SEQ ID NO: 188 - RNA, 19 nt, Artificial Sequence
  Synthetic Oligo
uuccacguagaagaccaga

SEQ ID NO: 189 - RNA, 19 nt, Artificial Sequence
  Synthetic Oligo
cuggucuucuacguggaaa

SEQ ID NO: 190 - RNA, 19 nt, Artificial Sequence
  Synthetic Oligo
uuuccacguagaagaccag

SEQ ID NO: 191 - RNA, 19 nt, Artificial Sequence
  Synthetic Oligo
uggucuucuacguggaaau

SEQ ID NO: 192 - RNA, 19 nt, Artificial Sequence
  Synthetic Oligo
auuuccacguagaagacca

SEQ ID NO: 193 - RNA, 19 nt, Artificial Sequence
  Synthetic Oligo
ggucuucuacguggaaaug

SEQ ID NO: 194 - RNA, 19 nt, Artificial Sequence
  Synthetic Oligo
cauuuccacguagaagacc

SEQ ID NO: 195 - RNA, 19 nt, Artificial Sequence
  Synthetic Oligo
gucuucuacguggaaauga

SEQ ID NO: 196 - RNA, 19 nt, Artificial Sequence
  Synthetic Oligo
ucauuuccacguagaagac

SEQ ID NO: 197 - RNA, 19 nt, Artificial Sequence
  Synthetic Oligo
ucuucuacguggaaaugaa

SEQ ID NO: 198 - RNA, 19 nt, Artificial Sequence
  Synthetic Oligo
uucauuuccacguagaaga

SEQ ID NO: 199 - RNA, 19 nt, Artificial Sequence
  Synthetic Oligo
cuucuacguggaaaugaac

SEQ ID NO: 200 - RNA, 19 nt, Artificial Sequence
  Synthetic Oligo
guucauuuccacguagaag

SEQ ID NO: 201 - RNA, 19 nt, Artificial Sequence
  Synthetic Oligo
uucuacguggaaaugaacg

SEQ ID NO: 202 - RNA, 19 nt, Artificial Sequence
  Synthetic Oligo
cguucauuuccacguagaa

SEQ ID NO: 203 - RNA, 19 nt, Artificial Sequence
  Synthetic Oligo
ucuacguggaaaugaacga

SEQ ID NO: 204 - RNA, 19 nt, Artificial Sequence
  Synthetic Oligo
ucguucauuuccacguaga

SEQ ID NO: 205 - RNA, 19 nt, Artificial Sequence
  Synthetic Oligo
cuacguggaaaugaacgag

SEQ ID NO: 206 - RNA, 19 nt, Artificial Sequence
  Synthetic Oligo
cucguucauuuccacguag

SEQ ID NO: 207 - RNA, 19 nt, Artificial Sequence
  Synthetic Oligo
uacguggaaaugaacgaga

SEQ ID NO: 208 - RNA, 19 nt, Artificial Sequence
  Synthetic Oligo
ucucguucauuuccacgua

SEQ ID NO: 209 - RNA, 19 nt, Artificial Sequence
  Synthetic Oligo
acguggaaaugaacgagag

SEQ ID NO: 210 - RNA, 19 nt, Artificial Sequence
  Synthetic Oligo
cucucguucauuuccacgu

SEQ ID NO: 211 - RNA, 19 nt, Artificial Sequence
  Synthetic Oligo
cguggaaaugaacgagagc

SEQ ID NO: 212 - RNA, 19 nt, Artificial Sequence
  Synthetic Oligo
gcucucguucauuuccacg

SEQ ID NO: 213 - RNA, 19 nt, Artificial Sequence
  Synthetic Oligo
guggaaaugaacgagagcc

SEQ ID NO: 214 - RNA, 19 nt, Artificial Sequence
  Synthetic Oligo
ggcucucguucauuuccac

SEQ ID NO: 215 - RNA, 19 nt, Artificial Sequence
  Synthetic Oligo
uggaaaugaacgagagcca

SEQ ID NO: 216 - RNA, 19 nt, Artificial Sequence
  Synthetic Oligo
uggcucucguucauuucca

SEQ ID NO: 217 - RNA, 19 nt, Artificial Sequence
  Synthetic Oligo
ggaaaugaacgagagccac

SEQ ID NO: 218 - RNA, 19 nt, Artificial Sequence
  Synthetic Oligo
guggcucucguucauuucc

SEQ ID NO: 219 - RNA, 19 nt, Artificial Sequence
  Synthetic Oligo
gaaaugaacgagagccaca

SEQ ID NO: 220 - RNA, 19 nt, Artificial Sequence
  Synthetic Oligo
uguggcucucguucauuuc

SEQ ID NO: 221 - RNA, 19 nt, Artificial Sequence
  Synthetic Oligo
aaaugaacgagagccacac

SEQ ID NO: 222 - RNA, 19 nt, Artificial Sequence
  Synthetic Oligo
guguggcucucguucauuu

SEQ ID NO: 223 - RNA, 19 nt, Artificial Sequence
  Synthetic Oligo
aaugaacgagagccacacg

SEQ ID NO: 224 - RNA, 19 nt, Artificial Sequence
  Synthetic Oligo
cguguggcucucguucauu

SEQ ID NO: 225 - RNA, 19 nt, Artificial Sequence
  Synthetic Oligo
augaacgagagccacacgc

SEQ ID NO: 226 - RNA, 19 nt, Artificial Sequence
  Synthetic Oligo
gcguguggcucucguucau

SEQ ID NO: 227 - RNA, 19 nt, Artificial Sequence
  Synthetic Oligo
ccacaggcauugccuccuu

SEQ ID NO: 228 - RNA, 19 nt, Artificial Sequence
  Synthetic Oligo
aaggaggcaaugccugugg

SEQ ID NO: 229 - RNA, 19 nt, Artificial Sequence
  Synthetic Oligo
acaggcauugccuccuuca

SEQ ID NO: 230 - RNA, 19 nt, Artificial Sequence
  Synthetic Oligo
ugaaggaggcaaugccugu

SEQ ID NO: 231 - RNA, 19 nt, Artificial Sequence
  Synthetic Oligo
caggcauugccuccuucac

SEQ ID NO: 232 - RNA, 19 nt, Artificial Sequence
  Synthetic Oligo
gugaaggaggcaaugccug

SEQ ID NO: 233 - RNA, 19 nt, Artificial Sequence
  Synthetic Oligo
aggcauugccuccuucacg

SEQ ID NO: 234 - RNA, 19 nt, Artificial Sequence
  Synthetic Oligo
cgugaaggaggcaaugccu

SEQ ID NO: 235 - RNA, 19 nt, Artificial Sequence
  Synthetic Oligo
gcauugccuccuucacgga

SEQ ID NO: 236 - RNA, 19 nt, Artificial Sequence
  Synthetic Oligo
uccgugaaggaggcaaugc

SEQ ID NO: 237 - RNA, 19 nt, Artificial Sequence
  Synthetic Oligo
cauugccuccuucacggag

SEQ ID NO: 238 - RNA, 19 nt, Artificial Sequence
  Synthetic Oligo
cuccgugaaggaggcaaug

SEQ ID NO: 239 - RNA, 19 nt, Artificial Sequence
  Synthetic Oligo
auugccuccuucacggaga

SEQ ID NO: 240 - RNA, 19 nt, Artificial Sequence
  Synthetic Oligo
ucuccgugaaggaggcaau

SEQ ID NO: 241 - RNA, 19 nt, Artificial Sequence
  Synthetic Oligo
uugccuccuucacggagag

SEQ ID NO: 242 - RNA, 19 nt, Artificial Sequence
  Synthetic Oligo
cucuccgugaaggaggcaa

SEQ ID NO: 243 - RNA, 19 nt, Artificial Sequence
  Synthetic Oligo
ugccuccuucacggagaga

SEQ ID NO: 244 - RNA, 19 nt, Artificial Sequence
  Synthetic Oligo
ucucuccgugaaggaggca

SEQ ID NO: 245 - RNA, 19 nt, Artificial Sequence
  Synthetic Oligo
ccuggcacacucaagacuc

SEQ ID NO: 246 - RNA, 19 nt, Artificial Sequence
  Synthetic Oligo
gagucuugagugugccagg

SEQ ID NO: 247 - RNA, 19 nt, Artificial Sequence
  Synthetic Oligo
cuggcacacucaagacucc

SEQ ID NO: 248 - RNA, 19 nt, Artificial Sequence
  Synthetic Oligo
ggagucuugagugugccag

SEQ ID NO: 249 - RNA, 19 nt, Artificial Sequence
  Synthetic Oligo
uggcacacucaagacuccc

SEQ ID NO: 250 - RNA, 19 nt, Artificial Sequence
  Synthetic Oligo
gggagucuugagugugcca

SEQ ID NO: 251 - RNA, 19 nt, Artificial Sequence
  Synthetic Oligo
ggcacacucaagacuccca

SEQ ID NO: 252 - RNA, 19 nt, Artificial Sequence
  Synthetic Oligo
ugggagucuugagugugcc

SEQ ID NO: 253 - RNA, 19 nt, Artificial Sequence
  Synthetic Oligo
gcacacucaagacucccac

SEQ ID NO: 254 - RNA, 19 nt, Artificial Sequence
  Synthetic Oligo
gugggagucuugagugugc

SEQ ID NO: 255 - RNA, 19 nt, Artificial Sequence
  Synthetic Oligo
cacacucaagacucccacg

SEQ ID NO: 256 - RNA, 19 nt, Artificial Sequence
  Synthetic Oligo
cgugggagucuugagugug

SEQ ID NO: 257 - RNA, 19 nt, Artificial Sequence
  Synthetic Oligo
acacucaagacucccacgg

SEQ ID NO: 258 - RNA, 19 nt, Artificial Sequence
  Synthetic Oligo
ccgugggagucuugagugu

SEQ ID NO: 259 - RNA, 19 nt, Artificial Sequence
  Synthetic Oligo
cacucaagacucccacgga

SEQ ID NO: 260 - RNA, 19 nt, Artificial Sequence
  Synthetic Oligo
uccgugggagucuugagug

SEQ ID NO: 261 - RNA, 19 nt, Artificial Sequence
  Synthetic Oligo
acucaagacucccacggag

SEQ ID NO: 262 - RNA, 19 nt, Artificial Sequence
  Synthetic Oligo
cuccgugggagucuugagu

SEQ ID NO: 263 - RNA, 19 nt, Artificial Sequence
  Synthetic Oligo
ucaagacucccacggaggu

SEQ ID NO: 264 - RNA, 19 nt, Artificial Sequence
  Synthetic Oligo
accuccgugggagucuuga

SEQ ID NO: 265 - RNA, 19 nt, Artificial Sequence
  Synthetic Oligo
caagacucccacggagguu

SEQ ID NO: 266 - RNA, 19 nt, Artificial Sequence
  Synthetic Oligo
aaccuccgugggagucuug

SEQ ID NO: 267 - RNA, 19 nt, Artificial Sequence
  Synthetic Oligo
aagacucccacggagguuc

SEQ ID NO: 268 - RNA, 19 nt, Artificial Sequence
  Synthetic Oligo
gaaccuccgugggagucuu

SEQ ID NO: 269 - RNA, 19 nt, Artificial Sequence
  Synthetic Oligo
agacucccacggagguuca

SEQ ID NO: 270 - RNA, 19 nt, Artificial Sequence
  Synthetic Oligo
ugaaccuccgugggagucu

SEQ ID NO: 271 - RNA, 19 nt, Artificial Sequence
  Synthetic Oligo
acucccacggagguucagu

SEQ ID NO: 272 - RNA, 19 nt, Artificial Sequence
  Synthetic Oligo
acugaaccuccgugggagu

SEQ ID NO: 273 - RNA, 19 nt, Artificial Sequence
  Synthetic Oligo
cucccacggagguucaguu

SEQ ID NO: 274 - RNA, 19 nt, Artificial Sequence
  Synthetic Oligo
aacugaaccuccgugggag

SEQ ID NO: 275 - RNA, 19 nt, Artificial Sequence
  Synthetic Oligo
ucccacggagguucaguuc

SEQ ID NO: 276 - RNA, 19 nt, Artificial Sequence
  Synthetic Oligo
gaacugaaccuccguggga

SEQ ID NO: 277 - RNA, 19 nt, Artificial Sequence
  Synthetic Oligo
ccacggagguucaguucca

SEQ ID NO: 278 - RNA, 19 nt, Artificial Sequence
  Synthetic Oligo
uggaacugaaccuccgugg

SEQ ID NO: 279 - RNA, 19 nt, Artificial Sequence
  Synthetic Oligo
cacggagguucaguuccac

SEQ ID NO: 280 - RNA, 19 nt, Artificial Sequence
  Synthetic Oligo
guggaacugaaccuccgug

SEQ ID NO: 281 - RNA, 19 nt, Artificial Sequence
  Synthetic Oligo
acggagguucaguuccaca

SEQ ID NO: 282 - RNA, 19 nt, Artificial Sequence
  Synthetic Oligo
uguggaacugaaccuccgu

SEQ ID NO: 283 - RNA, 19 nt, Artificial Sequence
  Synthetic Oligo
cggagguucaguuccacac

SEQ ID NO: 284 - RNA, 19 nt, Artificial Sequence
  Synthetic Oligo
guguggaacugaaccuccg

SEQ ID NO: 285 - RNA, 19 nt, Artificial Sequence
  Synthetic Oligo
ggagguucaguuccacacu

SEQ ID NO: 286 - RNA, 19 nt, Artificial Sequence
  Synthetic Oligo
aguguggaacugaaccucc

SEQ ID NO: 287 - RNA, 19 nt, Artificial Sequence
  Synthetic Oligo
gagguucaguuccacacuc

SEQ ID NO: 288 - RNA, 19 nt, Artificial Sequence
  Synthetic Oligo
gaguguggaacugaaccuc

SEQ ID NO: 289 - RNA, 19 nt, Artificial Sequence
  Synthetic Oligo
agguucaguuccacacucc

SEQ ID NO: 290 - RNA, 19 nt, Artificial Sequence
  Synthetic Oligo
ggaguguggaacugaaccu

SEQ ID NO: 291 - RNA, 19 nt, Artificial Sequence
  Synthetic Oligo
gguucaguuccacacuccc

SEQ ID NO: 292 - RNA, 19 nt, Artificial Sequence
  Synthetic Oligo
gggaguguggaacugaacc

SEQ ID NO: 293 - RNA, 19 nt, Artificial Sequence
  Synthetic Oligo
uuccucuucgucucuccgg

SEQ ID NO: 294 - RNA, 19 nt, Artificial Sequence
  Synthetic Oligo
ccggagagacgaagaggaa

SEQ ID NO: 295 - RNA, 19 nt, Artificial Sequence
  Synthetic Oligo
gcucaccgccauucaugaa

SEQ ID NO: 296 - RNA, 19 nt, Artificial Sequence
  Synthetic Oligo
uucaugaauggcggugagc

SEQ ID NO: 297 - RNA, 19 nt, Artificial Sequence
  Synthetic Oligo
ucaccgccauucaugaagg

SEQ ID NO: 298 - RNA, 19 nt, Artificial Sequence
  Synthetic Oligo
ccuucaugaauggcgguga

SEQ ID NO: 299 - RNA, 19 nt, Artificial Sequence
  Synthetic Oligo
caccgccauucaugaaggg

SEQ ID NO: 300 - RNA, 19 nt, Artificial Sequence
  Synthetic Oligo
cccuucaugaauggcggug

SEQ ID NO: 301 - RNA, 19 nt, Artificial Sequence
  Synthetic Oligo
ugccugugggccuuuacaa

SEQ ID NO: 302 - RNA, 19 nt, Artificial Sequence
  Synthetic Oligo
uuguaaaggcccacaggca

SEQ ID NO: 303 - RNA, 19 nt, Artificial Sequence
  Synthetic Oligo
gccugugggccuuuacaag

SEQ ID NO: 304 - RNA, 19 nt, Artificial Sequence
  Synthetic Oligo
cuuguaaaggcccacaggc

SEQ ID NO: 305 - RNA, 19 nt, Artificial Sequence
  Synthetic Oligo
ccugugggccuuuacaagg

SEQ ID NO: 306 - RNA, 19 nt, Artificial Sequence
  Synthetic Oligo
ccuuguaaaggcccacagg

SEQ ID NO: 307 - RNA, 19 nt, Artificial Sequence
  Synthetic Oligo
cugugggccuuuacaaggg

SEQ ID NO: 308 - RNA, 19 nt, Artificial Sequence
  Synthetic Oligo
cccuuguaaaggcccacag

SEQ ID NO: 309 - RNA, 19 nt, Artificial Sequence
  Synthetic Oligo
ugugggccuuuacaagggc

SEQ ID NO: 310 - RNA, 19 nt, Artificial Sequence
  Synthetic Oligo
gcccuuguaaaggcccaca

SEQ ID NO: 311 - RNA, 19 nt, Artificial Sequence
  Synthetic Oligo
cguccguccgugaaauucc

SEQ ID NO: 312 - RNA, 19 nt, Artificial Sequence
  Synthetic Oligo
ggaauuucacggacggacg

SEQ ID NO: 313 - RNA, 19 nt, Artificial Sequence
  Synthetic Oligo
guccguccgugaaauuccg

SEQ ID NO: 314 - RNA, 19 nt, Artificial Sequence
  Synthetic Oligo
cggaauuucacggacggac

SEQ ID NO: 315 - RNA, 19 nt, Artificial Sequence
  Synthetic Oligo
uccguccgugaaauuccgg

SEQ ID NO: 316 - RNA, 19 nt, Artificial Sequence
  Synthetic Oligo
ccggaauuucacggacgga

SEQ ID NO: 317 - RNA, 19 nt, Artificial Sequence
  Synthetic Oligo
gacggagacucguuuggac

SEQ ID NO: 318 - RNA, 19 nt, Artificial Sequence
  Synthetic Oligo
guccaaacgagucuccguc

SEQ ID NO: 319 - RNA, 19 nt, Artificial Sequence
  Synthetic Oligo
acggagacucguuuggacc

SEQ ID NO: 320 - RNA, 19 nt, Artificial Sequence
  Synthetic Oligo
gguccaaacgagucuccgu

SEQ ID NO: 321 - RNA, 19 nt, Artificial Sequence
  Synthetic Oligo
agccugcuuugagcggaac

SEQ ID NO: 322 - RNA, 19 nt, Artificial Sequence
  Synthetic Oligo
guuccgcucaaagcaggcu

SEQ ID NO: 323 - RNA, 19 nt, Artificial Sequence
  Synthetic Oligo
gcuuugagcggaacccgua

SEQ ID NO: 324 - RNA, 19 nt, Artificial Sequence
  Synthetic Oligo
uacggguuccgcucaaagc

SEQ ID NO: 325 - RNA, 19 nt, Artificial Sequence
  Synthetic Oligo
cuuugagcggaacccguac

SEQ ID NO: 326 - RNA, 19 nt, Artificial Sequence
  Synthetic Oligo
guacggguuccgcucaaag

SEQ ID NO: 327 - RNA, 19 nt, Artificial Sequence
  Synthetic Oligo
uuugagcggaacccguacc

SEQ ID NO: 328 - RNA, 19 nt, Artificial Sequence
  Synthetic Oligo
gguacggguuccgcucaaa

SEQ ID NO: 329 - RNA, 19 nt, Artificial Sequence
  Synthetic Oligo
cccaggguccagauuuggu

SEQ ID NO: 330 - RNA, 19 nt, Artificial Sequence
  Synthetic Oligo
accaaaucuggacccuggg

SEQ ID NO: 331 - RNA, 19 nt, Artificial Sequence
  Synthetic Oligo
ccaggguccagauuugguu

SEQ ID NO: 332 - RNA, 19 nt, Artificial Sequence
  Synthetic Oligo
aaccaaaucuggacccugg

SEQ ID NO: 333 - RNA, 19 nt, Artificial Sequence
  Synthetic Oligo
caggguccagauuugguuu

SEQ ID NO: 334 - RNA, 19 nt, Artificial Sequence
  Synthetic Oligo
aaaccaaaucuggacccug

SEQ ID NO: 335 - RNA, 19 nt, Artificial Sequence
  Synthetic Oligo
aggguccagauuugguuuc

SEQ ID NO: 336 - RNA, 19 nt, Artificial Sequence
  Synthetic Oligo
gaaaccaaaucuggacccu

SEQ ID NO: 337 - RNA, 19 nt, Artificial Sequence
  Synthetic Oligo
ggguccagauuugguuuca

SEQ ID NO: 338 - RNA, 19 nt, Artificial Sequence
  Synthetic Oligo
ugaaaccaaaucuggaccc

SEQ ID NO: 339 - RNA, 19 nt, Artificial Sequence
  Synthetic Oligo
gguccagauuugguuucag

SEQ ID NO: 340 - RNA, 19 nt, Artificial Sequence
  Synthetic Oligo
cugaaaccaaaucuggacc

SEQ ID NO: 341 - RNA, 19 nt, Artificial Sequence
  Synthetic Oligo
guccagauuugguuucaga

SEQ ID NO: 342 - RNA, 19 nt, Artificial Sequence
  Synthetic Oligo
ucugaaaccaaaucuggac

SEQ ID NO: 343 - RNA, 19 nt, Artificial Sequence
  Synthetic Oligo
uccagauuugguuucagaa

SEQ ID NO: 344 - RNA, 19 nt, Artificial Sequence
  Synthetic Oligo
uucugaaaccaaaucugga

SEQ ID NO: 345 - RNA, 19 nt, Artificial Sequence
  Synthetic Oligo
ccagauuugguuucagaau

SEQ ID NO: 346 - RNA, 19 nt, Artificial Sequence
  Synthetic Oligo
auucugaaaccaaaucugg

SEQ ID NO: 347 - RNA, 19 nt, Artificial Sequence
  Synthetic Oligo
cagauuugguuucagaaug

SEQ ID NO: 348 - RNA, 19 nt, Artificial Sequence
  Synthetic Oligo
cauucugaaaccaaaucug

SEQ ID NO: 349 - RNA, 19 nt, Artificial Sequence
  Synthetic Oligo
agauuugguuucagaauga

SEQ ID NO: 350 - RNA, 19 nt, Artificial Sequence
  Synthetic Oligo
ucauucugaaaccaaaucu

SEQ ID NO: 351 - RNA, 19 nt, Artificial Sequence
  Synthetic Oligo
gauuugguuucagaaugag

SEQ ID NO: 352 - RNA, 19 nt, Artificial Sequence
  Synthetic Oligo
cucauucugaaaccaaauc

SEQ ID NO: 353 - RNA, 19 nt, Artificial Sequence
  Synthetic Oligo
auuugguuucagaaugaga

SEQ ID NO: 354 - RNA, 19 nt, Artificial Sequence
  Synthetic Oligo
ucucauucugaaaccaaau

SEQ ID NO: 355 - RNA, 19 nt, Artificial Sequence
  Synthetic Oligo
uuugguuucagaaugagag

SEQ ID NO: 356 - RNA, 19 nt, Artificial Sequence
  Synthetic Oligo
cucucauucugaaaccaaa

SEQ ID NO: 357 - RNA, 19 nt, Artificial Sequence
  Synthetic Oligo
uugguuucagaaugagagg

SEQ ID NO: 358 - RNA, 19 nt, Artificial Sequence
  Synthetic Oligo
ccucucauucugaaaccaa

SEQ ID NO: 359 - RNA, 19 nt, Artificial Sequence
  Synthetic Oligo
ugguuucagaaugagaggu

SEQ ID NO: 360 - RNA, 19 nt, Artificial Sequence
  Synthetic Oligo
accucucauucugaaacca

SEQ ID NO: 361 - RNA, 19 nt, Artificial Sequence
  Synthetic Oligo
gguuucagaaugagagguc

SEQ ID NO: 362 - RNA, 19 nt, Artificial Sequence
  Synthetic Oligo
gaccucucauucugaaacc

SEQ ID NO: 363 - RNA, 19 nt, Artificial Sequence
  Synthetic Oligo
guuucagaaugagagguca

SEQ ID NO: 364 - RNA, 19 nt, Artificial Sequence
  Synthetic Oligo
ugaccucucauucugaaac

SEQ ID NO: 365 - RNA, 19 nt, Artificial Sequence
  Synthetic Oligo
uuucagaaugagaggucac

SEQ ID NO: 366 - RNA, 19 nt, Artificial Sequence
  Synthetic Oligo
gugaccucucauucugaaa

SEQ ID NO: 367 - RNA, 19 nt, Artificial Sequence
  Synthetic Oligo
uucagaaugagaggucacg

SEQ ID NO: 368 - RNA, 19 nt, Artificial Sequence
  Synthetic Oligo
cgugaccucucauucugaa

SEQ ID NO: 369 - RNA, 19 nt, Artificial Sequence
  Synthetic Oligo
ucagaaugagaggucacgc

SEQ ID NO: 370 - RNA, 19 nt, Artificial Sequence
  Synthetic Oligo
gcgugaccucucauucuga

SEQ ID NO: 371 - RNA, 19 nt, Artificial Sequence
  Synthetic Oligo
cagaaugagaggucacgcc

SEQ ID NO: 372 - RNA, 19 nt, Artificial Sequence
  Synthetic Oligo
ggcgugaccucucauucug

SEQ ID NO: 373 - RNA, 19 nt, Artificial Sequence
  Synthetic Oligo
agaaugagaggucacgcca

SEQ ID NO: 374 - RNA, 19 nt, Artificial Sequence
  Synthetic Oligo
uggcgugaccucucauucu

SEQ ID NO: 375 - RNA, 19 nt, Artificial Sequence
  Synthetic Oligo
gaaugagaggucacgccag

SEQ ID NO: 376 - RNA, 19 nt, Artificial Sequence
  Synthetic Oligo
cuggcgugaccucucauuc

SEQ ID NO: 377 - RNA, 19 nt, Artificial Sequence
  Synthetic Oligo
aaugagaggucacgccagc

SEQ ID NO: 378 - RNA, 19 nt, Artificial Sequence
  Synthetic Oligo
gcuggcgugaccucucauu

SEQ ID NO: 379 - RNA, 19 nt, Artificial Sequence
  Synthetic Oligo
augagaggucacgccagcu

SEQ ID NO: 380 - RNA, 19 nt, Artificial Sequence
  Synthetic Oligo
agcuggcgugaccucucau

SEQ ID NO: 381 - RNA, 19 nt, Artificial Sequence
  Synthetic Oligo
ugcuccuccgagccuuuga

SEQ ID NO: 382 - RNA, 19 nt, Artificial Sequence
  Synthetic Oligo
ucaaaggcucggaggagca

SEQ ID NO: 383 - RNA, 19 nt, Artificial Sequence
  Synthetic Oligo
uccuccgagccuuugagaa

SEQ ID NO: 384 - RNA, 19 nt, Artificial Sequence
  Synthetic Oligo
uucucaaaggcucggagga

SEQ ID NO: 385 - RNA, 19 nt, Artificial Sequence
  Synthetic Oligo
ccuccgagccuuugagaag

SEQ ID NO: 386 - RNA, 19 nt, Artificial Sequence
  Synthetic Oligo
cuucucaaaggcucggagg

SEQ ID NO: 387 - RNA, 19 nt, Artificial Sequence
  Synthetic Oligo
cuccgagccuuugagaagg

SEQ ID NO: 388 - RNA, 19 nt, Artificial Sequence
  Synthetic Oligo
ccuucucaaaggcucggag

SEQ ID NO: 389 - RNA, 19 nt, Artificial Sequence
  Synthetic Oligo
uccgagccuuugagaagga

SEQ ID NO: 390 - RNA, 19 nt, Artificial Sequence
  Synthetic Oligo
uccuucucaaaggcucgga

SEQ ID NO: 391 - RNA, 19 nt, Artificial Sequence
  Synthetic Oligo
ccgagccuuugagaaggau

SEQ ID NO: 392 - RNA, 19 nt, Artificial Sequence
  Synthetic Oligo
auccuucucaaaggcucgg

SEQ ID NO: 393 - RNA, 19 nt, Artificial Sequence
  Synthetic Oligo
cgagccuuugagaaggauc

SEQ ID NO: 394 - RNA, 19 nt, Artificial Sequence
  Synthetic Oligo
gauccuucucaaaggcucg

SEQ ID NO: 395 - RNA, 19 nt, Artificial Sequence
  Synthetic Oligo
gagccuuugagaaggaucg

SEQ ID NO: 396 - RNA, 19 nt, Artificial Sequence
  Synthetic Oligo
cgauccuucucaaaggcuc

SEQ ID NO: 397 - RNA, 19 nt, Artificial Sequence
  Synthetic Oligo
agccuuugagaaggaucgc

SEQ ID NO: 398 - RNA, 19 nt, Artificial Sequence
  Synthetic Oligo
gcgauccuucucaaaggcu

SEQ ID NO: 399 - RNA, 19 nt, Artificial Sequence
  Synthetic Oligo
gccuuugagaaggaucgcu

SEQ ID NO: 400 - RNA, 19 nt, Artificial Sequence
  Synthetic Oligo
agcgauccuucucaaaggc

SEQ ID NO: 401 - RNA, 19 nt, Artificial Sequence
  Synthetic Oligo
ccuuugagaaggaucgcuu

SEQ ID NO: 402 - RNA, 19 nt, Artificial Sequence
  Synthetic Oligo
aagcgauccuucucaaagg

SEQ ID NO: 403 - RNA, 19 nt, Artificial Sequence
  Synthetic Oligo
cuuugagaaggaucgcuuu

SEQ ID NO: 404 - RNA, 19 nt, Artificial Sequence
  Synthetic Oligo
aaagcgauccuucucaaag

SEQ ID NO: 405 - RNA, 19 nt, Artificial Sequence
  Synthetic Oligo
uuugagaaggaucgcuuuc

SEQ ID NO: 406 - RNA, 19 nt, Artificial Sequence
  Synthetic Oligo
gaaagcgauccuucucaaa

SEQ ID NO: 407 - RNA, 19 nt, Artificial Sequence
  Synthetic Oligo
uugagaaggaucgcuuucc

SEQ ID NO: 408 - RNA, 19 nt, Artificial Sequence
  Synthetic Oligo
ggaaagcgauccuucucaa

SEQ ID NO: 409 - RNA, 19 nt, Artificial Sequence
  Synthetic Oligo
ugagaaggaucgcuuucca

SEQ ID NO: 410 - RNA, 19 nt, Artificial Sequence
  Synthetic Oligo
uggaaagcgauccuucuca

SEQ ID NO: 411 - RNA, 19 nt, Artificial Sequence
  Synthetic Oligo
gagaaggaucgcuuuccag

SEQ ID NO: 412 - RNA, 19 nt, Artificial Sequence
  Synthetic Oligo
cuggaaagcgauccuucuc

SEQ ID NO: 413 - RNA, 19 nt, Artificial Sequence
  Synthetic Oligo
agaaggaucgcuuuccagg

SEQ ID NO: 414 - RNA, 19 nt, Artificial Sequence
  Synthetic Oligo
ccuggaaagcgauccuucu

SEQ ID NO: 415 - RNA, 19 nt, Artificial Sequence
  Synthetic Oligo
gaaggaucgcuuuccaggc

SEQ ID NO: 416 - RNA, 19 nt, Artificial Sequence
  Synthetic Oligo
gccuggaaagcgauccuuc

SEQ ID NO: 417 - RNA, 19 nt, Artificial Sequence
  Synthetic Oligo
aaggaucgcuuuccaggca

SEQ ID NO: 418 - RNA, 19 nt, Artificial Sequence
  Synthetic Oligo
ugccuggaaagcgauccuu

SEQ ID NO: 419 - RNA, 19 nt, Artificial Sequence
  Synthetic Oligo
aggaucgcuuuccaggcau

SEQ ID NO: 420 - RNA, 19 nt, Artificial Sequence
  Synthetic Oligo
augccuggaaagcgauccu

SEQ ID NO: 421 - RNA, 19 nt, Artificial Sequence
  Synthetic Oligo
ggaucgcuuuccaggcauc

SEQ ID NO: 422 - RNA, 19 nt, Artificial Sequence
  Synthetic Oligo
gaugccuggaaagcgaucc

SEQ ID NO: 423 - RNA, 19 nt, Artificial Sequence
  Synthetic Oligo
gaucgcuuuccaggcaucg

SEQ ID NO: 424 - RNA, 19 nt, Artificial Sequence
  Synthetic Oligo
cgaugccuggaaagcgauc

SEQ ID NO: 425 - RNA, 19 nt, Artificial Sequence
  Synthetic Oligo
ccggaguccaggauucaga

SEQ ID NO: 426 - RNA, 19 nt, Artificial Sequence
  Synthetic Oligo
ucugaauccuggacuccgg

SEQ ID NO: 427 - RNA, 19 nt, Artificial Sequence
  Synthetic Oligo
cggaguccaggauucagau

SEQ ID NO: 428 - RNA, 19 nt, Artificial Sequence
  Synthetic Oligo
aucugaauccuggacuccg

SEQ ID NO: 429 - RNA, 19 nt, Artificial Sequence
  Synthetic Oligo
ggaguccaggauucagauc

SEQ ID NO: 430 - RNA, 19 nt, Artificial Sequence
  Synthetic Oligo
gaucugaauccuggacucc

SEQ ID NO: 431 - RNA, 19 nt, Artificial Sequence
  Synthetic Oligo
gaguccaggauucagaucu

SEQ ID NO: 432 - RNA, 19 nt, Artificial Sequence
  Synthetic Oligo
agaucugaauccuggacuc

SEQ ID NO: 433 - RNA, 19 nt, Artificial Sequence
  Synthetic Oligo
aguccaggauucagaucug

SEQ ID NO: 434 - RNA, 19 nt, Artificial Sequence
  Synthetic Oligo
cagaucugaauccuggacu

SEQ ID NO: 435 - RNA, 19 nt, Artificial Sequence
  Synthetic Oligo
guccaggauucagaucugg

SEQ ID NO: 436 - RNA, 19 nt, Artificial Sequence
  Synthetic Oligo
ccagaucugaauccuggac

SEQ ID NO: 437 - RNA, 19 nt, Artificial Sequence
  Synthetic Oligo
uccaggauucagaucuggu

SEQ ID NO: 438 - RNA, 19 nt, Artificial Sequence
  Synthetic Oligo
accagaucugaauccugga

SEQ ID NO: 439 - RNA, 19 nt, Artificial Sequence
  Synthetic Oligo
ccaggauucagaucugguu

SEQ ID NO: 440 - RNA, 19 nt, Artificial Sequence
  Synthetic Oligo
aaccagaucugaauccugg

SEQ ID NO: 441 - RNA, 19 nt, Artificial Sequence
  Synthetic Oligo
caggauucagaucugguuu

SEQ ID NO: 442 - RNA, 19 nt, Artificial Sequence
  Synthetic Oligo
aaaccagaucugaauccug

SEQ ID NO: 443 - RNA, 19 nt, Artificial Sequence
  Synthetic Oligo
aggauucagaucugguuuc

SEQ ID NO: 444 - RNA, 19 nt, Artificial Sequence
  Synthetic Oligo
gaaaccagaucugaauccu

SEQ ID NO: 445 - RNA, 19 nt, Artificial Sequence
  Synthetic Oligo
ggauucagaucugguuuca

SEQ ID NO: 446 - RNA, 19 nt, Artificial Sequence
  Synthetic Oligo
ugaaaccagaucugaaucc

SEQ ID NO: 447 - RNA, 19 nt, Artificial Sequence
  Synthetic Oligo
gauucagaucugguuucag

SEQ ID NO: 448 - RNA, 19 nt, Artificial Sequence
  Synthetic Oligo
cugaaaccagaucugaauc

SEQ ID NO: 449 - RNA, 19 nt, Artificial Sequence
  Synthetic Oligo
auucagaucugguuucaga

SEQ ID NO: 450 - RNA, 19 nt, Artificial Sequence
  Synthetic Oligo
ucugaaaccagaucugaau

SEQ ID NO: 451 - RNA, 19 nt, Artificial Sequence
  Synthetic Oligo
uucagaucugguuucagaa

SEQ ID NO: 452 - RNA, 19 nt, Artificial Sequence
  Synthetic Oligo
uucugaaaccagaucugaa

SEQ ID NO: 453 - RNA, 19 nt, Artificial Sequence
  Synthetic Oligo
ucagaucugguuucagaau

SEQ ID NO: 454 - RNA, 19 nt, Artificial Sequence
  Synthetic Oligo
auucugaaaccagaucuga

SEQ ID NO: 455 - RNA, 19 nt, Artificial Sequence
  Synthetic Oligo
cagaucugguuucagaauc

SEQ ID NO: 456 - RNA, 19 nt, Artificial Sequence
  Synthetic Oligo
gauucugaaaccagaucug

SEQ ID NO: 457 - RNA, 19 nt, Artificial Sequence
  Synthetic Oligo
agaucugguuucagaaucg

SEQ ID NO: 458 - RNA, 19 nt, Artificial Sequence
  Synthetic Oligo
cgauucugaaaccagaucu

SEQ ID NO: 459 - RNA, 19 nt, Artificial Sequence
  Synthetic Oligo
gaucugguuucagaaucga

SEQ ID NO: 460 - RNA, 19 nt, Artificial Sequence
  Synthetic Oligo
ucgauucugaaaccagauc

SEQ ID NO: 461 - RNA, 19 nt, Artificial Sequence
  Synthetic Oligo
aucugguuucagaaucgaa

SEQ ID NO: 462 - RNA, 19 nt, Artificial Sequence
  Synthetic Oligo
uucgauucugaaaccagau

SEQ ID NO: 463 - RNA, 19 nt, Artificial Sequence
  Synthetic Oligo
ucugguuucagaaucgaag

SEQ ID NO: 464 - RNA, 19 nt, Artificial Sequence
  Synthetic Oligo
cuucgauucugaaaccaga

SEQ ID NO: 465 - RNA, 19 nt, Artificial Sequence
  Synthetic Oligo
cugguuucagaaucgaagg

SEQ ID NO: 466 - RNA, 19 nt, Artificial Sequence
  Synthetic Oligo
ccuucgauucugaaaccag

SEQ ID NO: 467 - RNA, 19 nt, Artificial Sequence
  Synthetic Oligo
ugguuucagaaucgaaggg

SEQ ID NO: 468 - RNA, 19 nt, Artificial Sequence
  Synthetic Oligo
cccuucgauucugaaacca

SEQ ID NO: 469 - RNA, 19 nt, Artificial Sequence
  Synthetic Oligo
gguuucagaaucgaagggc

SEQ ID NO: 470 - RNA, 19 nt, Artificial Sequence
  Synthetic Oligo
gcccuucgauucugaaacc

SEQ ID NO: 471 - RNA, 19 nt, Artificial Sequence
  Synthetic Oligo
guuucagaaucgaagggcc

SEQ ID NO: 472 - RNA, 19 nt, Artificial Sequence
  Synthetic Oligo
ggcccuucgauucugaaac

SEQ ID NO: 473 - RNA, 19 nt, Artificial Sequence
  Synthetic Oligo
uuucagaaucgaagggcca

SEQ ID NO: 474 - RNA, 19 nt, Artificial Sequence
  Synthetic Oligo
uggcccuucgauucugaaa

SEQ ID NO: 475 - RNA, 19 nt, Artificial Sequence
  Synthetic Oligo
uucagaaucgaagggccag

SEQ ID NO: 476 - RNA, 19 nt, Artificial Sequence
  Synthetic Oligo
cuggcccuucgauucugaa

SEQ ID NO: 477 - RNA, 19 nt, Artificial Sequence
  Synthetic Oligo
ucagaaucgaagggccagg

SEQ ID NO: 478 - RNA, 19 nt, Artificial Sequence
  Synthetic Oligo
ccuggcccuucgauucuga

SEQ ID NO: 479 - RNA, 19 nt, Artificial Sequence
  Synthetic Oligo
agaaucgaagggccaggca

SEQ ID NO: 480 - RNA, 19 nt, Artificial Sequence
  Synthetic Oligo
ugccuggcccuucgauucu

SEQ ID NO: 481 - RNA, 19 nt, Artificial Sequence
  Synthetic Oligo
aggcgcaaccucuccuaga

SEQ ID NO: 482 - RNA, 19 nt, Artificial Sequence
  Synthetic Oligo
ucuaggagagguugcgccu

SEQ ID NO: 483 - RNA, 19 nt, Artificial Sequence
  Synthetic Oligo
gcgcaaccucuccuagaaa

SEQ ID NO: 484 - RNA, 19 nt, Artificial Sequence
  Synthetic Oligo
uuucuaggagagguugcgc

SEQ ID NO: 485 - RNA, 19 nt, Artificial Sequence
  Synthetic Oligo
cgcaaccucuccuagaaac

SEQ ID NO: 486 - RNA, 19 nt, Artificial Sequence
  Synthetic Oligo
guuucuaggagagguugcg

SEQ ID NO: 487 - RNA, 19 nt, Artificial Sequence
  Synthetic Oligo
gcaaccucuccuagaaacg

SEQ ID NO: 488 - RNA, 19 nt, Artificial Sequence
  Synthetic Oligo
cguuucuaggagagguugc

SEQ ID NO: 489 - RNA, 19 nt, Artificial Sequence
  Synthetic Oligo
caaccucuccuagaaacgg

SEQ ID NO: 490 - RNA, 19 nt, Artificial Sequence
  Synthetic Oligo
ccguuucuaggagagguug

SEQ ID NO: 491 - RNA, 19 nt, Artificial Sequence
  Synthetic Oligo
aaccucuccuagaaacgga

SEQ ID NO: 492 - RNA, 19 nt, Artificial Sequence
  Synthetic Oligo
uccguuucuaggagagguu

SEQ ID NO: 493 - RNA, 19 nt, Artificial Sequence
  Synthetic Oligo
accucuccuagaaacggag

SEQ ID NO: 494 - RNA, 19 nt, Artificial Sequence
  Synthetic Oligo
cuccguuucuaggagaggu

SEQ ID NO: 495 - RNA, 19 nt, Artificial Sequence
  Synthetic Oligo
ccucuccuagaaacggagg

SEQ ID NO: 496 - RNA, 19 nt, Artificial Sequence
  Synthetic Oligo
ccuccguuucuaggagagg

SEQ ID NO: 497 - RNA, 19 nt, Artificial Sequence
  Synthetic Oligo
cucuccuagaaacggaggc

SEQ ID NO: 498 - RNA, 19 nt, Artificial Sequence
  Synthetic Oligo
gccuccguuucuaggagag

SEQ ID NO: 499 - RNA, 19 nt, Artificial Sequence
  Synthetic Oligo
ucuccuagaaacggaggcc

SEQ ID NO: 500 - RNA, 19 nt, Artificial Sequence
  Synthetic Oligo
ggccuccguuucuaggaga

SEQ ID NO: 501 - RNA, 19 nt, Artificial Sequence
  Synthetic Oligo
ucagcgaggaagaauaccg

SEQ ID NO: 502 - RNA, 19 nt, Artificial Sequence
  Synthetic Oligo
cgguauucuuccucgcuga

SEQ ID NO: 503 - RNA, 19 nt, Artificial Sequence
  Synthetic Oligo
cagcgaggaagaauaccgg

SEQ ID NO: 504 - RNA, 19 nt, Artificial Sequence
  Synthetic Oligo
ccgguauucuuccucgcug

SEQ ID NO: 505 - RNA, 19 nt, Artificial Sequence
  Synthetic Oligo
agcgaggaagaauaccggg

SEQ ID NO: 506 - RNA, 19 nt, Artificial Sequence
  Synthetic Oligo
cccgguauucuuccucgcu

SEQ ID NO: 507 - RNA, 19 nt, Artificial Sequence
  Synthetic Oligo
cgaggaagaauaccgggcu

SEQ ID NO: 508 - RNA, 19 nt, Artificial Sequence
  Synthetic Oligo
agcccgguauucuuccucg

SEQ ID NO: 509 - RNA, 19 nt, Artificial Sequence
  Synthetic Oligo
gaggaagaauaccgggcuc

SEQ ID NO: 510 - RNA, 19 nt, Artificial Sequence
  Synthetic Oligo
gagcccgguauucuuccuc

SEQ ID NO: 511 - RNA, 19 nt, Artificial Sequence
  Synthetic Oligo
aggaagaauaccgggcucu

SEQ ID NO: 512 - RNA, 19 nt, Artificial Sequence
  Synthetic Oligo
agagcccgguauucuuccu

SEQ ID NO: 513 - RNA, 19 nt, Artificial Sequence
  Synthetic Oligo
ggaagaauaccgggcucug

SEQ ID NO: 514 - RNA, 19 nt, Artificial Sequence
  Synthetic Oligo
cagagcccgguauucuucc

SEQ ID NO: 515 - RNA, 19 nt, Artificial Sequence
  Synthetic Oligo
gaagaauaccgggcucugc

SEQ ID NO: 516 - RNA, 19 nt, Artificial Sequence
  Synthetic Oligo
gcagagcccgguauucuuc

SEQ ID NO: 517 - RNA, 19 nt, Artificial Sequence
  Synthetic Oligo
aagaauaccgggcucugcu

SEQ ID NO: 518 - RNA, 19 nt, Artificial Sequence
  Synthetic Oligo
agcagagcccgguauucuu

SEQ ID NO: 519 - RNA, 19 nt, Artificial Sequence
  Synthetic Oligo
agaauaccgggcucugcug

SEQ ID NO: 520 - RNA, 19 nt, Artificial Sequence
  Synthetic Oligo
cagcagagcccgguauucu

SEQ ID NO: 521 - RNA, 19 nt, Artificial Sequence
  Synthetic Oligo
aauaccgggcucugcugga

SEQ ID NO: 522 - RNA, 19 nt, Artificial Sequence
  Synthetic Oligo
uccagcagagcccgguauu

SEQ ID NO: 523 - RNA, 19 nt, Artificial Sequence
  Synthetic Oligo
gcucugcuggaggagcuuu

SEQ ID NO: 524 - RNA, 19 nt, Artificial Sequence
  Synthetic Oligo
aaagcuccuccagcagagc

SEQ ID NO: 525 - RNA, 19 nt, Artificial Sequence
  Synthetic Oligo
cucugcuggaggagcuuua

SEQ ID NO: 526 - RNA, 19 nt, Artificial Sequence
  Synthetic Oligo
uaaagcuccuccagcagag

SEQ ID NO: 527 - RNA, 19 nt, Artificial Sequence
  Synthetic Oligo
ucugcuggaggagcuuuag

SEQ ID NO: 528 - RNA, 19 nt, Artificial Sequence
  Synthetic Oligo
cuaaagcuccuccagcaga

SEQ ID NO: 529 - RNA, 19 nt, Artificial Sequence
  Synthetic Oligo
cugcuggaggagcuuuagg

SEQ ID NO: 530 - RNA, 19 nt, Artificial Sequence
  Synthetic Oligo
ccuaaagcuccuccagcag

SEQ ID NO: 531 - RNA, 19 nt, Artificial Sequence
  Synthetic Oligo
ugcuggaggagcuuuagga

SEQ ID NO: 532 - RNA, 19 nt, Artificial Sequence
  Synthetic Oligo
uccuaaagcuccuccagca

SEQ ID NO: 533 - RNA, 19 nt, Artificial Sequence
  Synthetic Oligo
gcuggaggagcuuuaggac

SEQ ID NO: 534 - RNA, 19 nt, Artificial Sequence
  Synthetic Oligo
guccuaaagcuccuccagc

SEQ ID NO: 535 - RNA, 19 nt, Artificial Sequence
  Synthetic Oligo
cuggaggagcuuuaggacg

SEQ ID NO: 536 - RNA, 19 nt, Artificial Sequence
  Synthetic Oligo
cguccuaaagcuccuccag

SEQ ID NO: 537 - RNA, 19 nt, Artificial Sequence
  Synthetic Oligo
uggaggagcuuuaggacgc

SEQ ID NO: 538 - RNA, 19 nt, Artificial Sequence
  Synthetic Oligo
gcguccuaaagcuccucca

SEQ ID NO: 539 - RNA, 19 nt, Artificial Sequence
  Synthetic Oligo
ccugggauuccugccuucu

SEQ ID NO: 540 - RNA, 19 nt, Artificial Sequence
  Synthetic Oligo
agaaggcaggaaucccagg

SEQ ID NO: 541 - RNA, 19 nt, Artificial Sequence
  Synthetic Oligo
ugggauuccugccuucuag

SEQ ID NO: 542 - RNA, 19 nt, Artificial Sequence
  Synthetic Oligo
cuagaaggcaggaauccca

SEQ ID NO: 543 - RNA, 19 nt, Artificial Sequence
  Synthetic Oligo
gggauuccugccuucuagg

SEQ ID NO: 544 - RNA, 19 nt, Artificial Sequence
  Synthetic Oligo
ccuagaaggcaggaauccc

SEQ ID NO: 545 - RNA, 19 nt, Artificial Sequence
  Synthetic Oligo
ggauuccugccuucuaggu

SEQ ID NO: 546 - RNA, 19 nt, Artificial Sequence
  Synthetic Oligo
accuagaaggcaggaaucc

SEQ ID NO: 547 - RNA, 19 nt, Artificial Sequence
  Synthetic Oligo
gauuccugccuucuagguc

SEQ ID NO: 548 - RNA, 19 nt, Artificial Sequence
  Synthetic Oligo
gaccuagaaggcaggaauc

SEQ ID NO: 549 - RNA, 19 nt, Artificial Sequence
  Synthetic Oligo
auuccugccuucuaggucu

SEQ ID NO: 550 - RNA, 19 nt, Artificial Sequence
  Synthetic Oligo
agaccuagaaggcaggaau

SEQ ID NO: 551 - RNA, 19 nt, Artificial Sequence
  Synthetic Oligo
uuccugccuucuaggucua

SEQ ID NO: 552 - RNA, 19 nt, Artificial Sequence
  Synthetic Oligo
uagaccuagaaggcaggaa

SEQ ID NO: 553 - RNA, 19 nt, Artificial Sequence
  Synthetic Oligo
uccugccuucuaggucuag

SEQ ID NO: 554 - RNA, 19 nt, Artificial Sequence
  Synthetic Oligo
cuagaccuagaaggcagga

SEQ ID NO: 555 - RNA, 19 nt, Artificial Sequence
  Synthetic Oligo
ccugccuucuaggucuagg

SEQ ID NO: 556 - RNA, 19 nt, Artificial Sequence
  Synthetic Oligo
ccuagaccuagaaggcagg

SEQ ID NO: 557 - RNA, 19 nt, Artificial Sequence
  Synthetic Oligo
cugccuucuaggucuaggc

SEQ ID NO: 558 - RNA, 19 nt, Artificial Sequence
  Synthetic Oligo
gccuagaccuagaaggcag

SEQ ID NO: 559 - RNA, 19 nt, Artificial Sequence
  Synthetic Oligo
ugccuucuaggucuaggcc

SEQ ID NO: 560 - RNA, 19 nt, Artificial Sequence
  Synthetic Oligo
ggccuagaccuagaaggca

SEQ ID NO: 561 - RNA, 19 nt, Artificial Sequence
  Synthetic Oligo
uucuaggucuaggcccggu

SEQ ID NO: 562 - RNA, 19 nt, Artificial Sequence
  Synthetic Oligo
accgggccuagaccuagaa

SEQ ID NO: 563 - RNA, 19 nt, Artificial Sequence
  Synthetic Oligo
cgcggagaacugccauucu

SEQ ID NO: 564 - RNA, 19 nt, Artificial Sequence
  Synthetic Oligo
agaauggcaguucuccgcg

SEQ ID NO: 565 - RNA, 19 nt, Artificial Sequence
  Synthetic Oligo
cggagaacugccauucuuu

SEQ ID NO: 566 - RNA, 19 nt, Artificial Sequence
  Synthetic Oligo
aaagaauggcaguucuccg

SEQ ID NO: 567 - RNA, 19 nt, Artificial Sequence
  Synthetic Oligo
ggagaacugccauucuuuc

SEQ ID NO: 568 - RNA, 19 nt, Artificial Sequence
  Synthetic Oligo
gaaagaauggcaguucucc

SEQ ID NO: 569 - RNA, 19 nt, Artificial Sequence
  Synthetic Oligo
gagaacugccauucuuucc

SEQ ID NO: 570 - RNA, 19 nt, Artificial Sequence
  Synthetic Oligo
ggaaagaauggcaguucuc

SEQ ID NO: 571 - RNA, 19 nt, Artificial Sequence
  Synthetic Oligo
agaacugccauucuuuccu

SEQ ID NO: 572 - RNA, 19 nt, Artificial Sequence
  Synthetic Oligo
aggaaagaauggcaguucu

SEQ ID NO: 573 - RNA, 19 nt, Artificial Sequence
  Synthetic Oligo
gaacugccauucuuuccug

SEQ ID NO: 574 - RNA, 19 nt, Artificial Sequence
  Synthetic Oligo
caggaaagaauggcaguuc

SEQ ID NO: 575 - RNA, 19 nt, Artificial Sequence
  Synthetic Oligo
aacugccauucuuuccugg

SEQ ID NO: 576 - RNA, 19 nt, Artificial Sequence
  Synthetic Oligo
ccaggaaagaauggcaguu

SEQ ID NO: 577 - RNA, 19 nt, Artificial Sequence
  Synthetic Oligo
acugccauucuuuccuggg

SEQ ID NO: 578 - RNA, 19 nt, Artificial Sequence
  Synthetic Oligo
cccaggaaagaauggcagu

SEQ ID NO: 579 - RNA, 19 nt, Artificial Sequence
  Synthetic Oligo
cugccauucuuuccugggc

SEQ ID NO: 580 - RNA, 19 nt, Artificial Sequence
  Synthetic Oligo
gcccaggaaagaauggcag

SEQ ID NO: 581 - RNA, 19 nt, Artificial Sequence
  Synthetic Oligo
ugccauucuuuccugggca

SEQ ID NO: 582 - RNA, 19 nt, Artificial Sequence
  Synthetic Oligo
ugcccaggaaagaauggca

SEQ ID NO: 583 - RNA, 19 nt, Artificial Sequence
  Synthetic Oligo
gccauucuuuccugggcau

SEQ ID NO: 584 - RNA, 19 nt, Artificial Sequence
  Synthetic Oligo
augcccaggaaagaauggc

SEQ ID NO: 585 - RNA, 19 nt, Artificial Sequence
  Synthetic Oligo
ccauucuuuccugggcauc

SEQ ID NO: 586 - RNA, 19 nt, Artificial Sequence
  Synthetic Oligo
gaugcccaggaaagaaugg

SEQ ID NO: 587 - RNA, 19 nt, Artificial Sequence
  Synthetic Oligo
cauucuuuccugggcaucc

SEQ ID NO: 588 - RNA, 19 nt, Artificial Sequence
  Synthetic Oligo
ggaugcccaggaaagaaug

SEQ ID NO: 589 - RNA, 19 nt, Artificial Sequence
  Synthetic Oligo
auucuuuccugggcauccc

SEQ ID NO: 590 - RNA, 19 nt, Artificial Sequence
  Synthetic Oligo
gggaugcccaggaaagaau

SEQ ID NO: 591 - RNA, 19 nt, Artificial Sequence
  Synthetic Oligo
uucuuuccugggcaucccg

SEQ ID NO: 592 - RNA, 19 nt, Artificial Sequence
  Synthetic Oligo
cgggaugcccaggaaagaa

SEQ ID NO: 593 - DNA, 8671 bp, Homo sapiens
gggatgcgcgcgcctggggctctcccacagggggctttcgtgagccaggcagcgagggccgcccccgcgctgcagcccagccaggccgcgacggcagagggggtctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacgcctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccggggacggacggggggaggaaggcagggaggaaaagcggtcctcggcctccgggagtagcgggacccccgccctccgggaaaacggtcagcgtccggcgcgggctgagggctgggcccacagccgccgcgccggccggcgcggcacccattcgccccggttccgtggcccagggagtgggcggtttcctccgggacaaaagaccgggactcgggttgccgtcgggttttcacccgcgcggttcacagaccgcacatccccaggctgagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcatggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctccccgttcctcttcgtctctccggccccaccaccaccaccgccaccacgccctcccccaccaccccccccccccccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctgggtggctggctgtccgggcaggccccctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttggcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccgggggacggggggaggaaggcagggaggaaaagcggtcctcggcctccgggagtagcgggacccccgccctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggctcagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcacggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctcccccttcctcttcgtctctccggccccaccaccaccaccgccaccacgccctccccccccaccccccccccccaccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctggctggctggctgtccgggcaggcctcctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggccgtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagcctcccagctgccagcgcggagctcctggcggtcaaaagcatacctctgtctgtctttgcccgcttcctggctagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatagaagatttgcatcttttgtgtgatgagtgcagagatatgtcacaatatcccctgtagaaaaagcctgaaattggtttacataacttcggtgatcagtgcagatgtgtttcagaactccatagtagactgaacctagagaatggttacatcacttaggtgatcagtgtagagatatgttaaaattctcgtgtagacagagcctagacaattgttacatcacctagtgatcagtgcagggataagtcataaagcctcctgtaggcagagtgtaggcaagtgttccctccctgggctgatcagtgcagagatatctcacaaagcccctataagccaaaccttgacaagggttacatcacctgtttgagcagtggaaatatatatcacaaagccccctgtagacaaagcccagacaatttttacatctcctgagtgagcattggagagatctgtcacaatgcccctgtaggcagagcttagacaagtgttacatcacctgggtgatcagtgcagagatatgtcaaaacgctcctgtagtctgaacctagacaggagttacatcaccttggggatcagtgcagagatacgtgagaattcc

SEQ ID NO: 594 - DNA, 16 bp, Artificial Sequence
  Primer
gccggcccaggtacca

SEQ ID NO: 595 - DNA, 17 bp, Artificial Sequence
  Primer
cagcgagctcccttgca

SEQ ID NO: 596 - DNA, 13 bp, Artificial Sequence
  Probe
agtgcgcaccccg

SEQ ID NO: 597 - DNA, 21 bp, Artificial Sequence
  Synthetic siRNA
  misc_feature (1)..(1): Locked Nucleic Acid (LNA)
  misc_feature (5)..(5): 2′ O-Methyl RNA base
  misc_feature (10)..(10): 2′ O-Methyl RNA base
  misc_feature (12)..(12): 2′ O-Methyl RNA base
  misc_feature (14)..(14): 2′ O-Methyl RNA base
  misc_feature (20)..(21): Locked Nucleic Acid (LNA)
gaaaugaacgagagccacatt

SEQ ID NO: 598 - DNA, 21 bp, Artificial Sequence
  Synthetic siRNA
  misc_feature (2)..(2): 2′ O-Methyl RNA base
  misc_feature (4)..(4): 2′ O-Methyl RNA base
  misc_feature (7)..(7): 2′ O-Methyl RNA base
  misc_feature (9)..(9): 2′ O-Methyl RNA base
  misc_feature (11)..(11): 2′ O-Methyl RNA base
  misc_feature (13)..(14): 2′ O-Methyl RNA base
  misc_feature (16)..(16): 2′ O-Methyl RNA base
  misc_feature (18)..(18): 2′ O-Methyl RNA base
uguggcucucguucauuuctt

SEQ ID NO: 599 - DNA, 21 bp, Artificial Sequence
  Synthetic siRNA
  misc_feature (1)..(1): Locked Nucleic Acid (LNA)
  misc_feature (3)..(3): 2′ O-Methyl RNA base
  misc_feature (9)..(11): 2′ O-Methyl RNA base
  misc_feature (14)..(15): 2′ O-Methyl RNA base
  misc_feature (18)..(18): 2′ O-Methyl RNA base
  misc_feature (20)..(21): Locked Nucleic Acid (LNA)
gguccagauuugguuucagtt

SEQ ID NO: 600 - DNA, 21 bp, Artificial Sequence
  Synthetic siRNA
  misc_feature (2)..(2): 2′ O-Methyl RNA base
  misc_feature (12)..(12): 2′ O-Methyl RNA base
  misc_feature (14)..(14): 2′ O-Methyl RNA base
  misc_feature (16)..(16): 2′ O-Methyl RNA base
cugaaaccaaaucuggacctt

SEQ ID NO: 601 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (8)..(8): 2′ O-Methyl RNA base
  misc_feature (10)..(10): 2′ O-Methyl RNA base
  misc_feature (13)..(13): 2′ O-Methyl RNA base
  misc_feature (15)..(15): 2′ O-Methyl RNA base
  misc_feature (18)..(18): 2′ O-Methyl RNA base
  misc_feature (20)..(21): Locked Nucleic Acid (LNA)
guccagauuugguuucagatt

SEQ ID NO: 602 - DNA, 21 bp, Artificial Sequence
  Synthetic siRNA
  misc_feature (3)..(3): 2′ O-Methyl RNA base
  misc_feature (13)..(13): 2′ O-Methyl RNA base
  misc_feature (15)..(15): 2′ O-Methyl RNA base
  misc_feature (17)..(17): 2′ O-Methyl RNA base
ucugaaaccaaaucuggactt

SEQ ID NO: 603 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (6)..(6): 2′ O-Methyl RNA base
  misc_feature (8)..(8): 2′ O-Methyl RNA base
  misc_feature (10)..(10): 2′ O-Methyl RNA base
  misc_feature (14)..(14): 2′ O-Methyl RNA base
  misc_feature (17)..(17): 2′ O-Methyl RNA base
  misc_feature (20)..(21): Locked Nucleic Acid (LNA)
agauuugguuucagaaugatt

SEQ ID NO: 604 - DNA, 21 bp, Artificial Sequence
  Synthetic siRNA
  misc_feature (5)..(5): 2′ O-Methyl RNA base
  misc_feature (7)..(7): 2′ O-Methyl RNA base
  misc_feature (17)..(17): 2′ O-Methyl RNA base
  misc_feature (19)..(19): 2′ O-Methyl RNA base
ucauucugaaaccaaaucutt

SEQ ID NO: 605 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (7)..(7): 2′ O-Methyl RNA base
  misc_feature (9)..(9): 2′ O-Methyl RNA base
  misc_feature (12)..(12): 2′ O-Methyl RNA base
  misc_feature (15)..(16): 2′ O-Methyl RNA base
  misc_feature (18)..(18): 2′ O-Methyl RNA base
  misc_feature (20)..(21): Locked Nucleic Acid (LNA)
auuugguuucagaaugagatt

SEQ ID NO: 606 - DNA, 21 bp, Artificial Sequence
  Synthetic siRNA
  misc_feature (3)..(3): 2′ O-Methyl RNA base
  misc_feature (6)..(6): 2′ O-Methyl RNA base
  misc_feature (9)..(9): 2′ O-Methyl RNA base
  misc_feature (19)..(19): 2′ O-Methyl RNA base
ucucauucugaaaccaaautt

SEQ ID NO: 607 - DNA, 20 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (7)..(7): 2′ O-Methyl RNA base
  misc_feature (10)..(10): 2′ O-Methyl RNA base
  misc_feature (13)..(13): 2′ O-Methyl RNA base
  misc_feature (15)..(16): 2′ O-Methyl RNA base
  misc_feature (19)..(20): Locked Nucleic Acid (LNA)
guuucagaaugagagucatt

SEQ ID NO: 608 - DNA, 21 bp, Artificial Sequence
  Synthetic siRNA
  misc_feature (2)..(2): 2′ O-Methyl RNA base
  misc_feature (6)..(6): 2′ O-Methyl RNA base
  misc_feature (8)..(8): 2′ O-Methyl RNA base
  misc_feature (12)..(12): 2′ O-Methyl RNA base
  misc_feature (15)..(15): 2′ O-Methyl RNA base
ugaccucucauucugaaactt

SEQ ID NO: 609 - DNA, 21 bp, Artificial Sequence
  Synthetic siRNA
  misc_feature (1)..(1): Locked Nucleic Acid (LNA)
  misc_feature (4)..(4): 2′ O-Methyl RNA base
  misc_feature (6)..(7): 2′ O-Methyl RNA base
  misc_feature (9)..(9): 2′ O-Methyl RNA base
  misc_feature (12)..(12): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
gccuuugagaaggaucgcutt

SEQ ID NO: 610 - DNA, 21 bp, Artificial Sequence
  Synthetic siRNA
  misc_feature (2)..(2): 2′ O-Methyl RNA base
  misc_feature (4)..(4): 2′ O-Methyl RNA base
  misc_feature (6)..(6): 2′ O-Methyl RNA base
  misc_feature (9)..(9): 2′ O-Methyl RNA base
  misc_feature (12)..(12): 2′ O-Methyl RNA base
  misc_feature (17)..(17): 2′ O-Methyl RNA base
agcgauccuucucaaaggctt

SEQ ID NO: 611 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(5): 2′ O-Methyl RNA base
  misc_feature (7)..(7): 2′ O-Methyl RNA base
  misc_feature (10)..(11): 2′ O-Methyl RNA base
  misc_feature (13)..(13): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
cuuugagaaggaucgcuuutt

SEQ ID NO: 612 - DNA, 21 bp, Artificial Sequence
  Synthetic siRNA
  misc_feature (4)..(4): 2′ O-Methyl RNA base
  misc_feature (6)..(6): 2′ O-Methyl RNA base
  misc_feature (8)..(8): 2′ O-Methyl RNA base
  misc_feature (11)..(12): 2′ O-Methyl RNA base
  misc_feature (14)..(14): 2′ O-Methyl RNA base
  misc_feature (19)..(19): 2′ O-Methyl RNA base
aaagcgauccuucucaaagtt

SEQ ID NO: 613 - DNA, 21 bp, Artificial Sequence
  Synthetic siRNA
  misc_feature (1)..(1): Locked Nucleic Acid (LNA)
  misc_feature (3)..(4): 2′ O-Methyl RNA base
  misc_feature (7)..(7): 2′ O-Methyl RNA base
  misc_feature (9)..(9): 2′ O-Methyl RNA base
  misc_feature (11)..(11): 2′ O-Methyl RNA base
  misc_feature (14)..(14): 2′ O-Methyl RNA base
  misc_feature (16)..(16): 2′ O-Methyl RNA base
  misc_feature (19)..(19): 2′ O-Methyl RNA base
  misc_feature (20)..(21): Locked Nucleic Acid (LNA)
gauucagaucugguuucagtt

SEQ ID NO: 614 - DNA, 21 bp, Artificial Sequence
  Synthetic siRNA
  misc_feature (2)..(2): 2′ O-Methyl RNA base
  misc_feature (8)..(8): 2′ O-Methyl RNA base
  misc_feature (12)..(12): 2′ O-Methyl RNA base
  misc_feature (14)..(14): 2′ O-Methyl RNA base
  misc_feature (18)..(18): 2′ O-Methyl RNA base
cugaaaccagaucugaauctt

SEQ ID NO: 615 - DNA, 21 bp, Artificial Sequence
  Synthetic siRNA
  misc_feature (1)..(1): Locked Nucleic Acid (LNA)
  misc_feature (2)..(3): 2′ O-Methyl RNA base
  misc_feature (6)..(6): 2′ O-Methyl RNA base
  misc_feature (8)..(8): 2′ O-Methyl RNA base
  misc_feature (10)..(10): 2′ O-Methyl RNA base
  misc_feature (13)..(15): 2′ O-Methyl RNA base
  misc_feature (18)..(18): 2′ O-Methyl RNA base
  misc_feature (20)..(21): Locked Nucleic Acid (LNA)
auucagaucugguuucagatt

SEQ ID NO: 616 - DNA, 21 bp, Artificial Sequence
  Synthetic siRNA
  misc_feature (3)..(3): 2′ O-Methyl RNA base
  misc_feature (11)..(11): 2′ O-Methyl RNA base
  misc_feature (13)..(13): 2′ O-Methyl RNA base
  misc_feature (15)..(15): 2′ O-Methyl RNA base
  misc_feature (19)..(19): 2′ O-Methyl RNA base
ucugaaaccagaucugaautt

SEQ ID NO: 617 - DNA, 21 bp, Artificial Sequence
  Synthetic siRNA
  misc_feature (1)..(1): Locked Nucleic Acid (LNA)
  misc_feature (5)..(5): 2′ O-Methyl RNA base
  misc_feature (10)..(10): 2′ O-Methyl RNA base
  misc_feature (12)..(12): 2′ O-Methyl RNA base
  misc_feature (14)..(14): 2′ O-Methyl RNA base
  misc_feature (20)..(21): Locked Nucleic Acid (LNA)
gaaaugaacgagagccacatt

SEQ ID NO: 618 - DNA, 21 bp, Artificial Sequence
  Synthetic siRNA
  misc_feature (1)..(4): 2′ O-Methyl RNA base
  misc_feature (7)..(7): 2′ O-Methyl RNA base
  misc_feature (9)..(9): 2′ O-Methyl RNA base
  misc_feature (11)..(14): 2′ O-Methyl RNA base
  misc_feature (16)..(18): 2′ O-Methyl RNA base
  misc_feature (20)..(21): Locked Nucleic Acid (LNA)
uguggcucucguucauuuctt

SEQ ID NO: 619 - DNA, 21 bp, Artificial Sequence
  Synthetic siRNA
  misc_feature (1)..(1): Locked Nucleic Acid (LNA)
  misc_feature (3)..(3): 2′ O-Methyl RNA base
  misc_feature (9)..(11): 2′ O-Methyl RNA base
  misc_feature (14)..(16): 2′ O-Methyl RNA base
  misc_feature (20)..(21): Locked Nucleic Acid (LNA)
gguccagauuugguuucagtt

SEQ ID NO: 620 - DNA, 21 bp, Artificial Sequence
  Synthetic siRNA
  misc_feature (2)..(2): 2′ O-Methyl RNA base
  misc_feature (12)..(12): 2′ O-Methyl RNA base
  misc_feature (14)..(14): 2′ O-Methyl RNA base
  misc_feature (16)..(16): 2′ O-Methyl RNA base
  misc_feature (20)..(21): Locked Nucleic Acid (LNA)
cugaaaccaaaucuggacctt

SEQ ID NO: 621 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (8)..(10): 2′ O-Methyl RNA base
  misc_feature (13)..(15): 2′ O-Methyl RNA base
  misc_feature (18)..(18): 2′ O-Methyl RNA base
  misc_feature (20)..(21): Locked Nucleic Acid (LNA)
guccagauuugguuucagatt

SEQ ID NO: 622 - DNA, 21 bp, Artificial Sequence
  Synthetic siRNA
  misc_feature (1)..(1): 2′ O-Methyl RNA base
  misc_feature (3)..(3): 2′ O-Methyl RNA base
  misc_feature (13)..(13): 2′ O-Methyl RNA base
  misc_feature (15)..(15): 2′ O-Methyl RNA base
  misc_feature (17)..(17): 2′ O-Methyl RNA base
  misc_feature (20)..(21): Locked Nucleic Acid (LNA)
ucugaaaccaaaucuggactt

SEQ ID NO: 623 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(6): 2′ O-Methyl RNA base
  misc_feature (8)..(11): 2′ O-Methyl RNA base
  misc_feature (14)..(14): 2′ O-Methyl RNA base
  misc_feature (17)..(17): 2′ O-Methyl RNA base
  misc_feature (20)..(21): Locked Nucleic Acid (LNA)
agauuugguuucagaaugatt

SEQ ID NO: 624 - DNA, 21 bp, Artificial Sequence
  Synthetic siRNA
  misc_feature (1)..(1): 2′ O-Methyl RNA base
  misc_feature (4)..(5): 2′ O-Methyl RNA base
  misc_feature (7)..(7): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
ucauucugaaaccaaaucutt

SEQ ID NO: 625 - DNA, 21 bp, Artificial Sequence
  Synthetic siRNA
  misc_feature (1)..(1): Locked Nucleic Acid (LNA)
  misc_feature (2)..(4): 2′ O-Methyl RNA base
  misc_feature (7)..(9): 2′ O-Methyl RNA base
  misc_feature (12)..(12): 2′ O-Methyl RNA base
  misc_feature (15)..(16): 2′ O-Methyl RNA base
  misc_feature (18)..(18): 2′ O-Methyl RNA base
  misc_feature (20)..(21): Locked Nucleic Acid (LNA)
auuugguuucagaaugagatt

SEQ ID NO: 626 - DNA, 21 bp, Artificial Sequence
  Synthetic siRNA
  misc_feature (1)..(1): 2′ O-Methyl RNA base
  misc_feature (3)..(3): 2′ O-Methyl RNA base
  misc_feature (6)..(7): 2′ O-Methyl RNA base
  misc_feature (9)..(10): 2′ O-Methyl RNA base
  misc_feature (19)..(19): 2′ O-Methyl RNA base
  misc_feature (20)..(21): Locked Nucleic Acid (LNA)
ucucauucugaaaccaaautt

SEQ ID NO: 627 - DNA, 20 bp, Artificial Sequence
  Synthetic siRNA
  misc_feature (1)..(1): Locked Nucleic Acid (LNA)
  misc_feature (2)..(4): 2′ O-Methyl RNA base
  misc_feature (7)..(7): 2′ O-Methyl RNA base
  misc_feature (10)..(10): 2′ O-Methyl RNA base
  misc_feature (13)..(13): 2′ O-Methyl RNA base
  misc_feature (15)..(16): 2′ O-Methyl RNA base
  misc_feature (19)..(20): Locked Nucleic Acid (LNA)
guuucagaaugagagucatt

SEQ ID NO: 628 - DNA, 21 bp, Artificial Sequence
  Synthetic siRNA
  misc_feature (1)..(2): 2′ O-Methyl RNA base
  misc_feature (6)..(6): 2′ O-Methyl RNA base
  misc_feature (8)..(8): 2′ O-Methyl RNA base
  misc_feature (11)..(12): 2′ O-Methyl RNA base
  misc_feature (14)..(15): 2′ O-Methyl RNA base
  misc_feature (20)..(21): Locked Nucleic Acid (LNA)
ugaccucucauucugaaactt

SEQ ID NO: 629 - DNA, 21 bp, Artificial Sequence
  Synthetic siRNA
  misc_feature (1)..(1): Locked Nucleic Acid (LNA)
  misc_feature (4)..(7): 2′ O-Methyl RNA base
  misc_feature (9)..(9): 2′ O-Methyl RNA base
  misc_feature (12)..(12): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
gccuuugagaaggaucgcutt

SEQ ID NO: 630 - DNA, 21 bp, Artificial Sequence
  Synthetic siRNA
  misc_feature (2)..(2): 2′ O-Methyl RNA base
  misc_feature (4)..(4): 2′ O-Methyl RNA base
  misc_feature (6)..(6): 2′ O-Methyl RNA base
  misc_feature (9)..(10): 2′ O-Methyl RNA base
  misc_feature (12)..(12): 2′ O-Methyl RNA base
  misc_feature (17)..(17): 2′ O-Methyl RNA base
  misc_feature (20)..(21): Locked Nucleic Acid (LNA)
agcgauccuucucaaaggctt

SEQ ID NO: 631 - DNA, 21 bp, Artificial Sequence
  Synthetic siRNA
  misc_feature (1)..(1): Locked Nucleic Acid (LNA)
  misc_feature (2)..(5): 2′ O-Methyl RNA base
  misc_feature (7)..(7): 2′ O-Methyl RNA base
  misc_feature (10)..(11): 2′ O-Methyl RNA base
  misc_feature (13)..(13): 2′ O-Methyl RNA base
  misc_feature (15)..(15): 2′ O-Methyl RNA base
  misc_feature (17)..(19): 2′ O-Methyl RNA base
  misc_feature (20)..(21): Locked Nucleic Acid (LNA)
cuuugagaaggaucgcuuutt

SEQ ID NO: 632 - DNA, 21 bp, Artificial Sequence
  Synthetic siRNA
  misc_feature (4)..(4): 2′ O-Methyl RNA base
  misc_feature (6)..(6): 2′ O-Methyl RNA base
  misc_feature (8)..(8): 2′ O-Methyl RNA base
  misc_feature (11)..(12): 2′ O-Methyl RNA base
  misc_feature (14)..(14): 2′ O-Methyl RNA base
  misc_feature (19)..(19): 2′ O-Methyl RNA base
  misc_feature (20)..(21): Locked Nucleic Acid (LNA)
aaagcgauccuucucaaagtt

SEQ ID NO: 633 - DNA, 21 bp, Artificial Sequence
  Synthetic siRNA
  misc_feature (1)..(1): Locked Nucleic Acid (LNA)
  misc_feature (3)..(4): 2′ O-Methyl RNA base
  misc_feature (7)..(7): 2′ O-Methyl RNA base
  misc_feature (9)..(9): 2′ O-Methyl RNA base
  misc_feature (11)..(11): 2′ O-Methyl RNA base
  misc_feature (14)..(16): 2′ O-Methyl RNA base
  misc_feature (19)..(19): 2′ O-Methyl RNA base
  misc_feature (20)..(21): Locked Nucleic Acid (LNA)
gauucagaucugguuucagtt

SEQ ID NO: 634 - DNA, 21 bp, Artificial Sequence
  Synthetic siRNA
  misc_feature (2)..(2): 2′ O-Methyl RNA base
  misc_feature (10)..(10): 2′ O-Methyl RNA base
  misc_feature (12)..(12): 2′ O-Methyl RNA base
  misc_feature (14)..(14): 2′ O-Methyl RNA base
  misc_feature (18)..(18): 2′ O-Methyl RNA base
  misc_feature (20)..(21): Locked Nucleic Acid (LNA)
cugaaaccagaucugaauctt

SEQ ID NO: 635 - DNA, 21 bp, Artificial Sequence
  Synthetic siRNA
  misc_feature (1)..(1): Locked Nucleic Acid (LNA)
  misc_feature (2)..(3): 2′ O-Methyl RNA base
  misc_feature (6)..(6): 2′ O-Methyl RNA base
  misc_feature (8)..(8): 2′ O-Methyl RNA base
  misc_feature (10)..(10): 2′ O-Methyl RNA base
  misc_feature (13)..(15): 2′ O-Methyl RNA base
  misc_feature (18)..(18): 2′ O-Methyl RNA base
  misc_feature (20)..(21): Locked Nucleic Acid (LNA)
auucagaucugguuucagatt

SEQ ID NO: 636 - DNA, 21 bp, Artificial Sequence
  Synthetic siRNA
  misc_feature (1)..(1): 2′ O-Methyl RNA base
  misc_feature (3)..(3): 2′ O-Methyl RNA base
  misc_feature (11)..(11): 2′ O-Methyl RNA base
  misc_feature (13)..(13): 2′ O-Methyl RNA base
  misc_feature (15)..(15): 2′ O-Methyl RNA base
  misc_feature (19)..(19): 2′ O-Methyl RNA base
  misc_feature (20)..(21): Locked Nucleic Acid (LNA)
ucugaaaccagaucugaautt

SEQ ID NO: 637 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (8)..(8): 2′ O-Methyl RNA base
  misc_feature (10)..(10): 2′ O-Methyl RNA base
  misc_feature (13)..(13): 2′ O-Methyl RNA base
  misc_feature (15)..(15): 2′ O-Methyl RNA base
  misc_feature (18)..(18): 2′ O-Methyl RNA base
  misc_feature (20)..(21): Locked Nucleic Acid (LNA)
  misc_feature (20)..(21): Locked Nucleic Acid (LNA)
guccagauuugguuucagatt

SEQ ID NO: 638 - DNA, 21 bp, Artificial Sequence
  Synthetic siRNA
  misc_feature (3)..(3): 2′ O-Methyl RNA base
  misc_feature (13)..(13): 2′ O-Methyl RNA base
  misc_feature (15)..(15): 2′ O-Methyl RNA base
  misc_feature (17)..(17): 2′ O-Methyl RNA base
ucugaaaccaaaucuggactt

SEQ ID NO: 639 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (6)..(6): 2′ O-Methyl RNA base
  misc_feature (8)..(8): 2′ O-Methyl RNA base
  misc_feature (10)..(10): 2′ O-Methyl RNA base
  misc_feature (14)..(14): 2′ O-Methyl RNA base
  misc_feature (17)..(17): 2′ O-Methyl RNA base
  misc_feature (20)..(21): Locked Nucleic Acid (LNA)
agauuugguuucagaaugatt

SEQ ID NO: 640 - DNA, 21 bp, Artificial Sequence
  Synthetic siRNA
  misc_feature (5)..(5): 2′ O-Methyl RNA base
  misc_feature (7)..(7): 2′ O-Methyl RNA base
  misc_feature (17)..(17): 2′ O-Methyl RNA base
  misc_feature (19)..(19): 2′ O-Methyl RNA base
ucauucugaaaccaaaucutt

SEQ ID NO: 641 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (7)..(7): 2′ O-Methyl RNA base
  misc_feature (9)..(9): 2′ O-Methyl RNA base
  misc_feature (12)..(12): 2′ O-Methyl RNA base
  misc_feature (15)..(16): 2′ O-Methyl RNA base
  misc_feature (18)..(18): 2′ O-Methyl RNA base
  misc_feature (20)..(21): Locked Nucleic Acid (LNA)
auuugguuucagaaugagatt

SEQ ID NO: 642 - DNA, 21 bp, Artificial Sequence
  Synthetic siRNA
  misc_feature (3)..(3): 2′ O-Methyl RNA base
  misc_feature (6)..(6): 2′ O-Methyl RNA base
  misc_feature (9)..(9): 2′ O-Methyl RNA base
  misc_feature (19)..(19): 2′ O-Methyl RNA base
ucucauucugaaaccaaautt

SEQ ID NO: 643 - DNA, 21 bp, Artificial Sequence
  Synthetic siRNA
  misc_feature (1)..(1): Locked Nucleic Acid (LNA)
  misc_feature (4)..(4): 2′ O-Methyl RNA base
  misc_feature (6)..(7): 2′ O-Methyl RNA base
  misc_feature (9)..(9): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
gccuuugagaaggaucgcutt

SEQ ID NO: 644 - DNA, 21 bp, Artificial Sequence
  Synthetic siRNA
  misc_feature (2)..(2): 2′ O-Methyl RNA base
  misc_feature (9)..(9): 2′ O-Methyl RNA base
  misc_feature (12)..(12): 2′ O-Methyl RNA base
  misc_feature (17)..(17): 2′ O-Methyl RNA base
agcgauccuucucaaaggctt

SEQ ID NO: 645 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(5): 2′ O-Methyl RNA base
  misc_feature (10)..(15): 2′ O-Methyl RNA base
  misc_feature (18)..(19): 2′ O-Methyl RNA base
  misc_feature (20)..(21): Locked Nucleic Acid (LNA)
cuuugagaaggaucgcuuutt

SEQ ID NO: 646 - DNA, 21 bp, Artificial Sequence
  Synthetic siRNA
  misc_feature (4)..(4): 2′ O-Methyl RNA base
  misc_feature (12)..(12): 2′ O-Methyl RNA base
  misc_feature (14)..(14): 2′ O-Methyl RNA base
  misc_feature (19)..(19): 2′ O-Methyl RNA base
aaagcgauccuucucaaagtt

SEQ ID NO: 647 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(5): 2′ O-Methyl RNA base
  misc_feature (10)..(15): 2′ O-Methyl RNA base
  misc_feature (18)..(19): 2′ O-Methyl RNA base
  misc_feature (20)..(21): Locked Nucleic Acid (LNA)
cuuugagaaggaucgcuuutt

SEQ ID NO: 648 - DNA, 21 bp, Artificial Sequence
  Synthetic siRNA
  misc_feature (4)..(4): 2′ O-Methyl RNA base
  misc_feature (12)..(12): 2′ O-Methyl RNA base
  misc_feature (19)..(19): 2′ O-Methyl RNA base
aaagcgauccuucucaaagtt

SEQ ID NO: 649 - DNA, 21 bp, Artificial Sequence
  Synthetic siRNA
  misc_feature (1)..(1): Locked Nucleic Acid (LNA)
  misc_feature (5)..(5): 2′ O-Methyl RNA base
  misc_feature (10)..(10): 2′ O-Methyl RNA base
  misc_feature (12)..(12): 2′ O-Methyl RNA base
  misc_feature (14)..(14): 2′ O-Methyl RNA base
  misc_feature (20)..(21): Locked Nucleic Acid (LNA)
gaaaugaacgagagccacatt

SEQ ID NO: 650 - DNA, 21 bp, Artificial Sequence
  Synthetic siRNA
  misc_feature (2)..(2): 2′ O-Methyl RNA base
  misc_feature (4)..(4): 2′ O-Methyl RNA base
  misc_feature (7)..(7): 2′ O-Methyl RNA base
  misc_feature (9)..(9): 2′ O-Methyl RNA base
  misc_feature (11)..(11): 2′ O-Methyl RNA base
  misc_feature (13)..(14): 2′ O-Methyl RNA base
  misc_feature (16)..(16): 2′ O-Methyl RNA base
  misc_feature (18)..(18): 2′ O-Methyl RNA base
uguggcucucguucauuuctt

SEQ ID NO: 651 - DNA, 21 bp, Artificial Sequence
  Synthetic siRNA
  misc_feature (5)..(5): 2′ O-Methyl RNA base
  misc_feature (10)..(10): 2′ O-Methyl RNA base
  misc_feature (12)..(12): 2′ O-Methyl RNA base
  misc_feature (14)..(14): 2′ O-Methyl RNA base
gaaaugaacgagagccacatt

SEQ ID NO: 652 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (7)..(7): 2′ O-Methyl RNA base
  misc_feature (9)..(9): 2′ O-Methyl RNA base
  misc_feature (11)..(11): 2′ O-Methyl RNA base
  misc_feature (13)..(14): 2′ O-Methyl RNA base
  misc_feature (16)..(16): 2′ O-Methyl RNA base
  misc_feature (18)..(18): 2′ O-Methyl RNA base
  misc_feature (20)..(21): Locked Nucleic Acid (LNA)
tguggcucucguucauuuctt

SEQ ID NO: 653 - DNA, 21 bp, Artificial Sequence
  Synthetic siRNA
  misc_feature (1)..(1): Locked Nucleic Acid (LNA)
  misc_feature (5)..(5): 2′ O-Methyl RNA base
  misc_feature (8)..(10): 2′ O-Methyl RNA base
  misc_feature (14)..(14): 2′ O-Methyl RNA base
  misc_feature (18)..(18): 2′ O-Methyl RNA base
  misc_feature (20)..(21): Locked Nucleic Acid (LNA)
gaacggagggaaagacagatt

SEQ ID NO: 654 - DNA, 21 bp, Artificial Sequence
  Synthetic siRNA
  misc_feature (1)..(1): 2′ O-Methyl RNA base
  misc_feature (3)..(3): 2′ O-Methyl RNA base
  misc_feature (5)..(5): 2′ O-Methyl RNA base
  misc_feature (8)..(9): 2′ O-Methyl RNA base
  misc_feature (13)..(13): 2′ O-Methyl RNA base
  misc_feature (16)..(16): 2′ O-Methyl RNA base
  misc_feature (18)..(18): 2′ O-Methyl RNA base
ucugucuuucccuccguuctt

SEQ ID NO: 655 - DNA, 21 bp, Artificial Sequence
  Synthetic siRNA
  misc_feature (5)..(5): 2′ O-Methyl RNA base
  misc_feature (8)..(10): 2′ O-Methyl RNA base
  misc_feature (14)..(14): 2′ O-Methyl RNA base
  misc_feature (18)..(18): 2′ O-Methyl RNA base
gaacggagggaaagacagatt

SEQ ID NO: 656 - DNA, 21 bp, Artificial Sequence
  Synthetic siRNA
  misc_feature (1)..(1): Locked Nucleic Acid (LNA)
  misc_feature (3)..(3): 2′ O-Methyl RNA base
  misc_feature (5)..(5): 2′ O-Methyl RNA base
  misc_feature (8)..(9): 2′ O-Methyl RNA base
  misc_feature (13)..(13): 2′ O-Methyl RNA base
  misc_feature (16)..(16): 2′ O-Methyl RNA base
  misc_feature (18)..(18): 2′ O-Methyl RNA base
  misc_feature (20)..(21): Locked Nucleic Acid (LNA)
tcugucuuucccuccguuctt

SEQ ID NO: 657 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (6)..(6): 2′ O-Methyl RNA base
  misc_feature (8)..(9): 2′ O-Methyl RNA base
  misc_feature (14)..(14): 2′ O-Methyl RNA base
  misc_feature (17)..(17): 2′ O-Methyl RNA base
  misc_feature (20)..(21): Locked Nucleic Acid (LNA)
auccaucgucaggccaucatt

SEQ ID NO: 658 - DNA, 21 bp, Artificial Sequence
  Synthetic siRNA
  misc_feature (1)..(1): 2′ O-Methyl RNA base
  misc_feature (4)..(4): 2′ O-Methyl RNA base
  misc_feature (7)..(7): 2′ O-Methyl RNA base
  misc_feature (9)..(9): 2′ O-Methyl RNA base
  misc_feature (13)..(13): 2′ O-Methyl RNA base
  misc_feature (15)..(15): 2′ O-Methyl RNA base
  misc_feature (19)..(19): 2′ O-Methyl RNA base
ugauggccugacgauggautt

SEQ ID NO: 659 - DNA, 21 bp, Artificial Sequence
  Synthetic siRNA
  misc_feature (2)..(2): 2′ O-Methyl RNA base
  misc_feature (6)..(6): 2′ O-Methyl RNA base
  misc_feature (8)..(9): 2′ O-Methyl RNA base
  misc_feature (14)..(14): 2′ O-Methyl RNA base
  misc_feature (17)..(17): 2′ O-Methyl RNA base
auccaucgucaggccaucatt

SEQ ID NO: 660 - DNA, 21 bp, Artificial Sequence
  Synthetic siRNA
  misc_feature (1)..(1): Locked Nucleic Acid (LNA)
  misc_feature (4)..(4): 2′ O-Methyl RNA base
  misc_feature (7)..(7): 2′ O-Methyl RNA base
  misc_feature (9)..(9): 2′ O-Methyl RNA base
  misc_feature (13)..(13): 2′ O-Methyl RNA base
  misc_feature (15)..(15): 2′ O-Methyl RNA base
  misc_feature (19)..(19): 2′ O-Methyl RNA base
  misc_feature (20)..(21): Locked Nucleic Acid (LNA)
tgauggccugacgauggautt

SEQ ID NO: 661 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (6)..(6): 2′ O-Methyl RNA base
  misc_feature (8)..(8): 2′ O-Methyl RNA base
  misc_feature (10)..(11): 2′ O-Methyl RNA base
  misc_feature (13)..(13): 2′ O-Methyl RNA base
  misc_feature (15)..(15): 2′ O-Methyl RNA base
  misc_feature (18)..(18): 2′ O-Methyl RNA base
  misc_feature (20)..(21): Locked Nucleic Acid (LNA)
guccagauuugguuucagatt

SEQ ID NO: 662 - DNA, 21 bp, Artificial Sequence
  Synthetic siRNA
  misc_feature (1)..(1): 5Phos
  misc_feature (3)..(3): 2′ O-Methyl RNA base
  misc_feature (6)..(6): 2′ O-Methyl RNA base
  misc_feature (11)..(11): 2′ O-Methyl RNA base
  misc_feature (13)..(13): 2′ O-Methyl RNA base
  misc_feature (15)..(15): 2′ O-Methyl RNA base
  misc_feature (17)..(17): 2′ O-Methyl RNA base
ucugaaaccaaaucuggactt

SEQ ID NO: 663 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (6)..(6): 2′ O-Methyl RNA base
  misc_feature (8)..(8): 2′ O-Methyl RNA base
  misc_feature (10)..(11): 2′ O-Methyl RNA base
  misc_feature (13)..(13): 2′ O-Methyl RNA base
  misc_feature (15)..(15): 2′ O-Methyl RNA base
  misc_feature (18)..(18): 2′ O-Methyl RNA base
  misc_feature (20)..(21): Locked Nucleic Acid (LNA)
guccagauuugguuucagatt

SEQ ID NO: 664 - DNA, 21 bp, Artificial Sequence
  Synthetic siRNA
  misc_feature (1)..(1): 5Phos
  misc_feature (3)..(3): 2′ O-Methyl RNA base
  misc_feature (6)..(6): 2′ O-Methyl RNA base
  misc_feature (11)..(11): 2′ O-Methyl RNA base
  misc_feature (13)..(13): 2′ O-Methyl RNA base
  misc_feature (15)..(15): 2′ O-Methyl RNA base
  misc_feature (17)..(17): 2′ O-Methyl RNA base
  misc_feature (20)..(21): Locked Nucleic Acid (LNA)
ucugaaaccaaaucuggactt

SEQ ID NO: 665 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (6)..(6): 2′ O-Methyl RNA base
  misc_feature (8)..(8): 2′ O-Methyl RNA base
  misc_feature (10)..(11): 2′ O-Methyl RNA base
  misc_feature (13)..(13): 2′ O-Methyl RNA base
  misc_feature (15)..(15): 2′ O-Methyl RNA base
  misc_feature (18)..(18): 2′ O-Methyl RNA base
  misc_feature (20)..(21): Locked Nucleic Acid (LNA)
guccagauuugguuucagatt

SEQ ID NO: 666 - DNA, 21 bp, Artificial Sequence
  Synthetic siRNA
  misc_feature (1)..(1): 5Phos
  misc_feature (3)..(3): 2′ O-Methyl RNA base
  misc_feature (6)..(6): 2′ O-Methyl RNA base
  misc_feature (11)..(11): 2′ O-Methyl RNA base
  misc_feature (13)..(13): 2′ O-Methyl RNA base
  misc_feature (15)..(15): 2′ O-Methyl RNA base
  misc_feature (17)..(17): 2′ O-Methyl RNA base
  misc_feature (20)..(21): Locked Nucleic Acid (LNA)
ucugaaaccaaaucuggactt

SEQ ID NO: 667 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (6)..(6): 2′ O-Methyl RNA base
  misc_feature (8)..(8): 2′ O-Methyl RNA base
  misc_feature (10)..(10): 2′ O-Methyl RNA base
  misc_feature (14)..(14): 2′ O-Methyl RNA base
  misc_feature (17)..(17): 2′ O-Methyl RNA base
  misc_feature (20)..(21): Locked Nucleic Acid (LNA)
agauuugguuucagaaugatt

SEQ ID NO: 668 - DNA, 21 bp, Artificial Sequence
  Synthetic siRNA
  misc_feature (1)..(1): 5Phos
  misc_feature (5)..(5): 2′ O-Methyl RNA base
  misc_feature (7)..(7): 2′ O-Methyl RNA base
  misc_feature (9)..(9): 2′ O-Methyl RNA base
  misc_feature (11)..(11): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
ucauucugaaaccaaaucutt

SEQ ID NO: 669 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (6)..(6): 2′ O-Methyl RNA base
  misc_feature (8)..(8): 2′ O-Methyl RNA base
  misc_feature (10)..(10): 2′ O-Methyl RNA base
  misc_feature (14)..(14): 2′ O-Methyl RNA base
  misc_feature (17)..(17): 2′ O-Methyl RNA base
  misc_feature (20)..(21): Locked Nucleic Acid (LNA)
agauuugguuucagaaugatt

SEQ ID NO: 670 - DNA, 21 bp, Artificial Sequence
  Synthetic siRNA
  misc_feature (1)..(1): 5Phos
  misc_feature (5)..(5): 2′ O-Methyl RNA base
  misc_feature (7)..(7): 2′ O-Methyl RNA base
  misc_feature (9)..(9): 2′ O-Methyl RNA base
  misc_feature (11)..(11): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
ucauucugaaaccaaaucutt

SEQ ID NO: 671 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (7)..(7): 2′ O-Methyl RNA base
  misc_feature (9)..(10): 2′ O-Methyl RNA base
  misc_feature (12)..(13): 2′ O-Methyl RNA base
  misc_feature (15)..(16): 2′ O-Methyl RNA base
  misc_feature (18)..(18): 2′ O-Methyl RNA base
  misc_feature (20)..(21): Locked Nucleic Acid (LNA)
auuugguuucagaaugagatt

SEQ ID NO: 672 - DNA, 21 bp, Artificial Sequence
  Synthetic siRNA
  misc_feature (1)..(1): 5Phos
  misc_feature (3)..(3): 2′ O-Methyl RNA base
  misc_feature (6)..(6): 2′ O-Methyl RNA base
  misc_feature (9)..(9): 2′ O-Methyl RNA base
  misc_feature (12)..(12): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
ucucauucugaaaccaaautt

SEQ ID NO: 673 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(4): 2′ O-Methyl RNA base
  misc_feature (7)..(7): 2′ O-Methyl RNA base
  misc_feature (9)..(10): 2′ O-Methyl RNA base
  misc_feature (12)..(13): 2′ O-Methyl RNA base
  misc_feature (15)..(16): 2′ O-Methyl RNA base
  misc_feature (18)..(18): 2′ O-Methyl RNA base
  misc_feature (20)..(21): Locked Nucleic Acid (LNA)
auuugguuucagaaugagatt

SEQ ID NO: 674 - DNA, 21 bp, Artificial Sequence
  Synthetic siRNA
  misc_feature (1)..(1): 5Phos
  misc_feature (3)..(3): 2′ O-Methyl RNA base
  misc_feature (6)..(6): 2′ O-Methyl RNA base
  misc_feature (9)..(9): 2′ O-Methyl RNA base
  misc_feature (12)..(12): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
ucucauucugaaaccaaautt

SEQ ID NO: 675 - DNA, 21 bp, Artificial Sequence
  Synthetic siRNA
  misc_feature (1)..(1): Locked Nucleic Acid (LNA)
  misc_feature (4)..(4): 2′ O-Methyl RNA base
  misc_feature (6)..(7): 2′ O-Methyl RNA base
  misc_feature (9)..(9): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
gccuuugagaaggaucgcutt

SEQ ID NO: 676 - DNA, 21 bp, Artificial Sequence
  Synthetic siRNA
  misc_feature (1)..(1): 5Phos
  misc_feature (2)..(2): 2′ O-Methyl RNA base
  misc_feature (6)..(6): 2′ O-Methyl RNA base
  misc_feature (9)..(9): 2′ O-Methyl RNA base
  misc_feature (12)..(12): 2′ O-Methyl RNA base
  misc_feature (15)..(15): 2′ O-Methyl RNA base
  misc_feature (17)..(17): 2′ O-Methyl RNA base
agcgauccuucucaaaggctt

SEQ ID NO: 677 - DNA, 21 bp, Artificial Sequence
  Synthetic siRNA
  misc_feature (1)..(1): Locked Nucleic Acid (LNA)
  misc_feature (4)..(4): 2′ O-Methyl RNA base
  misc_feature (6)..(7): 2′ O-Methyl RNA base
  misc_feature (9)..(9): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
gccuuugagaaggaucgcutt

SEQ ID NO: 678 - DNA, 21 bp, Artificial Sequence
  Synthetic siRNA
  misc_feature (1)..(1): 5Phos
  misc_feature (2)..(2): 2′ O-Methyl RNA base
  misc_feature (6)..(6): 2′ O-Methyl RNA base
  misc_feature (9)..(9): 2′ O-Methyl RNA base
  misc_feature (12)..(12): 2′ O-Methyl RNA base
  misc_feature (15)..(15): 2′ O-Methyl RNA base
  misc_feature (17)..(17): 2′ O-Methyl RNA base
  misc_feature (20)..(21): Locked Nucleic Acid (LNA)
agcgauccuucucaaaggctt

SEQ ID NO: 679 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(5): 2′ O-Methyl RNA base
  misc_feature (7)..(7): 2′ O-Methyl RNA base
  misc_feature (10)..(11): 2′ O-Methyl RNA base
  misc_feature (13)..(13): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
cuuugagaaggaucgcuuutt

SEQ ID NO: 680 - DNA, 21 bp, Artificial Sequence
  Synthetic siRNA
  misc_feature (1)..(1): 5Phos
  misc_feature (2)..(2): 2′ O-Methyl RNA base
  misc_feature (4)..(4): 2′ O-Methyl RNA base
  misc_feature (6)..(6): 2′ O-Methyl RNA base
  misc_feature (8)..(8): 2′ O-Methyl RNA base
  misc_feature (12)..(12): 2′ O-Methyl RNA base
  misc_feature (14)..(14): 2′ O-Methyl RNA base
  misc_feature (17)..(17): 2′ O-Methyl RNA base
  misc_feature (19)..(19): 2′ O-Methyl RNA base
aaagcgauccuucucaaagtt

SEQ ID NO: 681 - DNA, 21 bp, Artificial Sequence
  Synthetic siRNA
  misc_feature (1)..(1): Locked Nucleic Acid (LNA)
  misc_feature (2)..(2): 2′ O-Methyl RNA base
  misc_feature (4)..(5): 2′ O-Methyl RNA base
  misc_feature (7)..(7): 2′ O-Methyl RNA base
  misc_feature (10)..(11): 2′ O-Methyl RNA base
  misc_feature (13)..(13): 2′ O-Methyl RNA base
  misc_feature (15)..(15): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
cuuugagaaggaucgcuuutt

SEQ ID NO: 682 - DNA, 21 bp, Artificial Sequence
  Synthetic siRNA
  misc_feature (1)..(1): 5Phos
  misc_feature (2)..(2): 2′ O-Methyl RNA base
  misc_feature (4)..(4): 2′ O-Methyl RNA base
  misc_feature (6)..(6): 2′ O-Methyl RNA base
  misc_feature (8)..(8): 2′ O-Methyl RNA base
  misc_feature (12)..(12): 2′ O-Methyl RNA base
  misc_feature (14)..(14): 2′ O-Methyl RNA base
  misc_feature (17)..(17): 2′ O-Methyl RNA base
  misc_feature (19)..(19): 2′ O-Methyl RNA base
  misc_feature (20)..(21): Locked Nucleic Acid (LNA)
aaagcgauccuucucaaagtt

SEQ ID NO: 683 - DNA, 21 bp, Artificial Sequence
  Synthetic siRNA
  misc_feature (1)..(1): 2′-Fluoro
  misc_feature (2)..(2): 2′ O-Methyl RNA base
  misc_feature (3)..(3): 2′-Fluoro
  misc_feature (4)..(4): 2′ O-Methyl RNA base
  misc_feature (5)..(5): 2′-Fluoro
  misc_feature (6)..(6): 2′ O-Methyl RNA base
  misc_feature (7)..(7): 2′-Fluoro
  misc_feature (8)..(8): 2′ O-Methyl RNA base
  misc_feature (9)..(9): 2′-Fluoro
  misc_feature (10)..(10): 2′ O-Methyl RNA base
  misc_feature (11)..(11): 2′-Fluoro
  misc_feature (12)..(12): 2′ O-Methyl RNA base
  misc_feature (13)..(13): 2′-Fluoro
  misc_feature (14)..(14): 2′ O-Methyl RNA base
  misc_feature (15)..(15): 2′-Fluoro
  misc_feature (16)..(16): 2′ O-Methyl RNA base
  misc_feature (17)..(17): 2′-Fluoro
  misc_feature (18)..(18): 2′ O-Methyl RNA base
  misc_feature (19)..(19): 2′-Fluoro
  misc_feature (20)..(21): Locked Nucleic Acid (LNA)
agauuugguuucagaaugatt

SEQ ID NO: 684 - DNA, 21 bp, Artificial Sequence
  Synthetic siRNA
  misc_feature (1)..(1): 2′ O-Methyl RNA base
  misc_feature (2)..(2): 2′-Fluoro
  misc_feature (3)..(3): 2′ O-Methyl RNA base
  misc_feature (4)..(4): 2′-Fluoro
  misc_feature (5)..(5): 2′ O-Methyl RNA base
  misc_feature (6)..(6): 2′-Fluoro
  misc_feature (7)..(7): 2′ O-Methyl RNA base
  misc_feature (8)..(8): 2′-Fluoro
  misc_feature (9)..(9): 2′ O-Methyl RNA base
  misc_feature (10)..(10): 2′-Fluoro
  misc_feature (11)..(11): 2′ O-Methyl RNA base
  misc_feature (12)..(12): 2′-Fluoro
  misc_feature (13)..(13): 2′ O-Methyl RNA base
  misc_feature (14)..(14): 2′-Fluoro
  misc_feature (15)..(15): 2′ O-Methyl RNA base
  misc_feature (16)..(16): 2′-Fluoro
  misc_feature (17)..(17): 2′ O-Methyl RNA base
  misc_feature (18)..(18): 2′-Fluoro
  misc_feature (19)..(19): 2′ O-Methyl RNA base
  misc_feature (20)..(21): Locked Nucleic Acid (LNA)
ucauucugaaaccaaaucutt

SEQ ID NO: 685 - DNA, 21 bp, Artificial Sequence
  Synthetic siRNA
  misc_feature (1)..(1): 2′-Fluoro
  misc_feature (2)..(2): 2′ O-Methyl RNA base
  misc_feature (3)..(3): 2′-Fluoro
  misc_feature (4)..(4): 2′ O-Methyl RNA base
  misc_feature (5)..(5): 2′-Fluoro
  misc_feature (6)..(6): 2′ O-Methyl RNA base
  misc_feature (7)..(7): 2′-Fluoro
  misc_feature (8)..(8): 2′ O-Methyl RNA base
  misc_feature (9)..(9): 2′-Fluoro
  misc_feature (10)..(10): 2′ O-Methyl RNA base
  misc_feature (11)..(11): 2′-Fluoro
  misc_feature (12)..(12): 2′ O-Methyl RNA base
  misc_feature (13)..(13): 2′-Fluoro
  misc_feature (14)..(14): 2′ O-Methyl RNA base
  misc_feature (15)..(15): 2′-Fluoro
  misc_feature (16)..(16): 2′ O-Methyl RNA base
  misc_feature (17)..(17): 2′-Fluoro
  misc_feature (18)..(18): 2′ O-Methyl RNA base
  misc_feature (19)..(19): 2′-Fluoro
  misc_feature (20)..(21): Locked Nucleic Acid (LNA)
cuuugagaaggaucgcuuutt

SEQ ID NO: 686 - DNA, 21 bp, Artificial Sequence
  Synthetic siRNA
  misc_feature (1)..(1): 2′ O-Methyl RNA base
  misc_feature (2)..(2): 2′-Fluoro
  misc_feature (3)..(3): 2′ O-Methyl RNA base
  misc_feature (4)..(4): 2′-Fluoro
  misc_feature (5)..(5): 2′ O-Methyl RNA base
  misc_feature (6)..(6): 2′-Fluoro
  misc_feature (7)..(7): 2′ O-Methyl RNA base
  misc_feature (8)..(8): 2′-Fluoro
  misc_feature (9)..(9): 2′ O-Methyl RNA base
  misc_feature (10)..(10): 2′-Fluoro
  misc_feature (11)..(11): 2′ O-Methyl RNA base
  misc_feature (12)..(12): 2′-Fluoro
  misc_feature (13)..(13): 2′ O-Methyl RNA base
  misc_feature (14)..(14): 2′-Fluoro
  misc_feature (15)..(15): 2′ O-Methyl RNA base
  misc_feature (16)..(16): 2′-Fluoro
  misc_feature (17)..(17): 2′ O-Methyl RNA base
  misc_feature (18)..(18): 2′-Fluoro
  misc_feature (19)..(19): 2′ O-Methyl RNA base
aaagcgauccuucucaaagtt

SEQ ID NO: 687 - DNA, 21 bp, Artificial Sequence
  Synthetic siRNA
  misc_feature (1)..(1): 2′-Fluoro
  misc_feature (2)..(2): 2′ O-Methyl RNA base
  misc_feature (3)..(3): 2′-Fluoro
  misc_feature (4)..(4): 2′ O-Methyl RNA base
  misc_feature (5)..(5): 2′-Fluoro
  misc_feature (6)..(6): 2′ O-Methyl RNA base
  misc_feature (7)..(7): 2′-Fluoro
  misc_feature (8)..(8): 2′ O-Methyl RNA base
  misc_feature (9)..(9): 2′-Fluoro
  misc_feature (10)..(10): 2′ O-Methyl RNA base
  misc_feature (11)..(11): 2′-Fluoro
  misc_feature (12)..(12): 2′ O-Methyl RNA base
  misc_feature (13)..(13): 2′-Fluoro
  misc_feature (14)..(14): 2′ O-Methyl RNA base
  misc_feature (15)..(15): 2′-Fluoro
  misc_feature (16)..(16): 2′ O-Methyl RNA base
  misc_feature (17)..(17): 2′-Fluoro
  misc_feature (18)..(18): 2′ O-Methyl RNA base
  misc_feature (19)..(19): 2′-Fluoro
  misc_feature (20)..(21): Locked Nucleic Acid (LNA)
auucagaucugguuucagatt

SEQ ID NO: 688 - DNA, 21 bp, Artificial Sequence
  Synthetic siRNA
  misc_feature (1)..(1): 2′ O-Methyl RNA base
  misc_feature (2)..(2): 2′-Fluoro
  misc_feature (3)..(3): 2′ O-Methyl RNA base
  misc_feature (4)..(4): 2′-Fluoro
  misc_feature (5)..(5): 2′ O-Methyl RNA base
  misc_feature (6)..(6): 2′-Fluoro
  misc_feature (7)..(7): 2′ O-Methyl RNA base
  misc_feature (8)..(8): 2′-Fluoro
  misc_feature (9)..(9): 2′ O-Methyl RNA base
  misc_feature (10)..(10): 2′-Fluoro
  misc_feature (11)..(11): 2′ O-Methyl RNA base
  misc_feature (12)..(12): 2′-Fluoro
  misc_feature (13)..(13): 2′ O-Methyl RNA base
  misc_feature (14)..(14): 2′-Fluoro
  misc_feature (15)..(15): 2′ O-Methyl RNA base
  misc_feature (16)..(16): 2′-Fluoro
  misc_feature (17)..(17): 2′ O-Methyl RNA base
  misc_feature (18)..(18): 2′-Fluoro
  misc_feature (19)..(19): 2′ O-Methyl RNA base
ucugaaaccagaucugaautt

SEQ ID NO: 689 - DNA, 8 bp, Artificial Sequence
  Synthetic
atcgatcg

SEQ ID NO: 690 - RNA, 8 nt, Artificial Sequence
  Synthetic
aucgaucg

SEQ ID NO: 691 - DNA, 8 bp, Artificial Sequence
  Synthetic
aucgatcg

SEQ ID NO: 692 - DNA, 8 bp, Artificial Sequence
  Synthetic
  misc_feature (3)..(3): modified with a methyl group at the 5-position
atcgaucg